(19) United States
(12) Patent Application Publication
(10) Pub. No.: US 2025/0282759 A1
(43) Pub. Date: Sep. 11, 2025

(54) POLYMORPHIC AND SALT FORMS OF THE AUTOTAXIN INHIBITOR CUDETAXESTAT

(71) Applicant: Sabre Therapeutics LLC, San Fancisco, CA (US)

(72) Inventors: Prabha Ibrahim, Mountain View, CA (US); Jack Jong-Jye Lin, Hercules, CA (US)

(21) Appl. No.: 18/862,461

(22) PCT Filed: Apr. 28, 2023
(86) PCT No.: PCT/US2023/020428

§ 371 Date: Nov. 1, 2024

Related U.S. Application Data
(60) Provisional application No. 63/364,065, filed on May 3, 2022.

(51) Int. Cl. C07D 403/04; A61K 31/4155; C07C 215/10; C07C 279/14

(52) U.S. Cl. CPC C07D 403/04; A61K 31/4155; C07C 215/10; C07C 279/14

(57) ABSTRACT

Disclosed herein are autotaxin inhibitor compounds, polymorphic forms, pharmaceutical compositions, and the method of use and preparation thereof. Some embodiments relate to crystalline forms of Compound 1 and salts thereof.

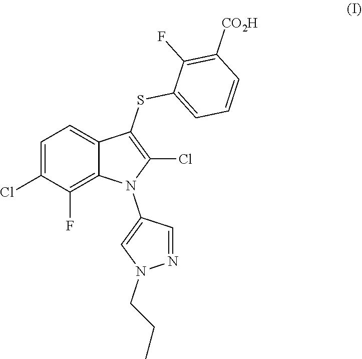 [SMILES: CCCn1cc(-n2c(Cl)c(Sc3cccc(C(=O)O)c3F)c3ccc(Cl)c(F)c32)cn1]

### BACKGROUND

### Field

The present application relates to the fields of chemistry and medicine. More particularly, the present application relates autotaxin inhibitors and salts, polymorphic forms, and pharmaceutical compositions thereof, and their use as therapeutic agents.

### Description of the Related Art

Autotaxin (ATX), also known as ectonucleotide pyrophosphatase/phosphodiesterase family member 2 or ENNP2, is an adipocyte secreted lysophospholipase D that catalyzes the formation of the lipid mediator, lysophosphatidic acid (LPA). Autotaxin expression is enhanced in individuals with certain conditions or diseases.

Autotaxin inhibitors may be useful as therapeutics for conditions such as a variety of fibrotic disease and disorders and cancers. Accordingly, a need exists to provide autotaxin compounds with suitable properties for therapeutic use.

### SUMMARY

Some embodiments provide a crystalline form a compound of Formula (I):

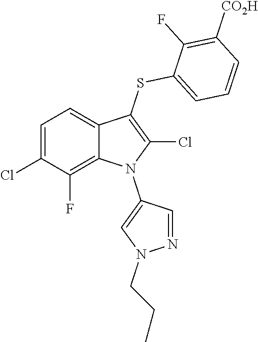 [SMILES: CCCn1cc(-n2c(Cl)c(Sc3cccc(C(=O)O)c3F)c3ccc(Cl)c(F)c32)cn1]

(I), or a solvate thereof.

In some embodiments, the crystalline form exhibits an X-ray powder diffraction pattern comprising at least one characteristic peak, wherein said characteristic peak is selected from the group consisting of approximately 7.2, 10.4, 12.0, 14.5, 17.9, 21.5, 21.8, 22.3, 23.5, 25.0, 25.8, 27.2, 27.7, 29.8, 31.6, and 36.7 degrees 2θ.

In some embodiments, the crystalline form exhibits an X-ray powder diffraction pattern comprising at least three characteristic peaks, wherein said characteristic peaks are selected from a group consisting of approximately 7.2, 10.4, 12.0, 14.5, 17.9, 21.5, 21.8, 22.3, 23.5, 25.0, 25.8, 27.2, 27.7, 29.8, 31.6, and 36.7 degrees 2θ.

In some embodiments, the crystalline form exhibits an X-ray powder diffraction pattern comprising characteristic peaks at approximately 7.2, 10.4, 17.9, 21.5, 21.8, and 25.8 degrees 2θ.

In some embodiments, the crystalline form exhibits an X-ray powder diffraction pattern comprising at least one characteristic peaks, wherein said characteristic peaks are selected from a group consisting of approximately 6.1, 7.1, 8.8, 10.5, 12.1, 14.0, 14.4, 15.0, 17.0, 18.3, 21.1, 21.9, 23.1, 24.1, 24.4, 25.4, 27.0, 27.5, and 28.2 degrees 2θ.

In some embodiments, the crystalline form exhibits an X-ray powder diffraction pattern comprising at least three characteristic peaks, wherein said characteristic peaks are selected from a group consisting of approximately 6.1, 7.1, 8.8, 10.5, 12.1, 14.0, 14.4, 15.0, 17.0, 18.3, 21.1, 21.9, 23.1, 24.1, 24.4, 25.4, 27.0, 27.5, and 28.2 degrees 2θ.

In some embodiments, the crystalline form exhibits an X-ray powder diffraction pattern comprising characteristic peaks at approximately 6.1, 7.1, 8.8, 14.4, 17.0, 18.3, 21.1, 21.9, 23.1, 25.4, and 28.2 degrees 2θ.

In some embodiments, the crystalline form exhibits an X-ray powder diffraction pattern comprising at least one characteristic peak, wherein said characteristic peak is selected from the group consisting of approximately 6.7, 9.6, 11.2, 13.3, 16.1, 17.7, 18.1, 18.9, 19.3, 20.0, 20.6, 21.5, 24.5, 24.9, 27.5, 28.3, 33.7, and 33.9 degrees 2θ.

In some embodiments, the crystalline form exhibits an X-ray diffraction powder diffraction pattern comprising at least three characteristic peaks, wherein said characteristic peaks are selected from a group consisting of approximately 6.7, 9.6, 11.2, 13.3, 16.1, 17.7, 18.1, 18.9, 19.3, 20.0, 20.6, 21.5, 24.5, 24.9, 27.5, 28.3, 33.7, and 33.9 degrees 2θ.

In some embodiments, the crystalline form exhibits an X-ray powder diffraction pattern comprising characteristic peaks at approximately 6.7, 11.2, 13.3, 16.1, 17.7, 18.1, 18.9, 19.3, 20.0, 20.6, 21.5, and 24.9 degrees 2θ.

In some embodiments, the crystalline form exhibits an X-ray powder diffraction pattern comprising at least one characteristic peaks, wherein said characteristic peaks are selected from a group consisting of approximately 6.7, 11.2, 12.0, 13.7, 16.0, 17.5, 17.9, 18.7, 20.0, 20.4, 21.4, 22.9, 24.1, 24.7, 25.1, 25.7, 26.1, 27.3, 27.7, 28.3, 28.8, 29.9, 30.3, 30.8, 33.8, 34.3, 35.4, and 38.1 degrees 2θ.

In some embodiments, the crystalline form exhibits an X-ray powder diffraction pattern comprising at least three characteristic peaks, wherein said characteristic peaks are selected from a group consisting of approximately 6.7, 11.2, 12.0, 13.7, 16.0, 17.5, 17.9, 18.7, 20.0, 20.4, 21.4, 22.9, 24.1, 24.7, 25.1, 25.7, 26.1, 27.3, 27.7, 28.3, 28.8, 29.9, 30.3, 30.8, 33.8, 34.3, 35.4, and 38.1 degrees 2θ.

In some embodiments, the crystalline form exhibits an X-ray powder diffraction pattern comprising characteristic peaks at approximately 6.7, 11.2, 12.0, 13.7, 16.0, 17.5, 17.9, 18.7, 20.4, 21.4, 22.9, 24.1, 24.7, 29.9, 30.3, and 30.8 degrees 2θ.

In some embodiments, the crystalline form exhibits an X-ray powder diffraction pattern comprising at least one characteristic peaks, wherein said characteristic peaks are selected from a group consisting of approximately 6.7, 7.3, 9.9, 12.0, 13.3, 15.0, 17.3, 18.6, 19.9, 20.5, 21.3, 23.2, 23.8, 24.6, 25.8, 26.3, 26.7, 28.3, 29.2, 29.8, 31.6, 33.5, 35.0, and 38.7 degrees 2θ.

In some embodiments, the crystalline form exhibits an X-ray powder diffraction pattern comprising at least three characteristic peaks, wherein said characteristic peaks are selected from a group consisting of approximately 6.7, 7.3, 9.9, 12.0, 13.3, 15.0, 17.3, 18.6, 19.9, 20.5, 21.3, 23.2, 23.8, 24.6, 25.8, 26.3, 26.7, 28.3, 29.2, 29.8, 31.6, 33.5, 35.0, and 38.7 degrees 2θ.

In some embodiments, the crystalline form exhibits an X-ray powder diffraction pattern comprising characteristic peaks at approximately 6.7, 9.9, 12.0, 13.3, 17.3, 18.6, 19.9, 23.2, 24.6, 26.3, 29.2, 29.8, 35.0, and 38.7 degrees 2θ.

In some embodiments, the crystalline form is a hydrate.

In some embodiments, the crystalline form is anhydrous.

In some embodiments, the crystalline form is a solvate.

In some embodiments, crystalline form is an ethanol solvate.

In some embodiments, the crystalline form is a chloroform solvate.

In some embodiments, greater than 50% by weight of the total amount of the compound of Formula (I) in the composition is the crystalline form.

In some embodiments, greater than 85% by weight of the total amount of the compound of Formula (I) in the composition is the crystalline form.

In some embodiments, greater than 90% by weight of the total amount of the compound of Formula (I) in the composition is the crystalline form.

Some embodiments provide a crystalline an arginine salt form of a compound of Formula (I):

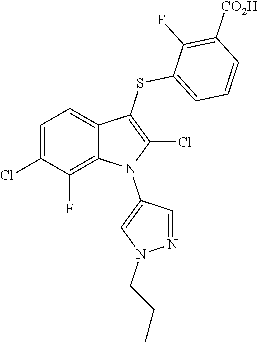 [SMILES: CCCn1cc(-n2c(Cl)c(Sc3cccc(C(=O)O)c3F)c3ccc(Cl)c(F)c32)cn1]

(I), or a solvate thereof.

In some embodiments, the arginine salt is in crystalline form.

In some embodiments, the arginine salt crystalline form exhibits an X-ray powder diffraction pattern comprising at least one characteristic peak, wherein said characteristic peak is selected from the group consisting of approximately 5.9, 8.8, 10.4, 12.3, 14.6, 17.1, 17.5, 19.4, 20.7, 25.7, 27.0, and 28.1 degrees 2θ.

In some embodiments, the arginine salt crystalline form exhibits an X-ray diffraction powder diffraction pattern comprising at least three characteristic peaks, wherein said characteristic peaks are selected from a group consisting of approximately 5.9, 8.8, 10.4, 12.3, 14.6, 17.1, 17.5, 19.4, 20.7, 25.7, 27.0, and 28.1 degrees 2θ.

In some embodiments, the arginine salt crystalline form exhibits an X-ray powder diffraction pattern comprising characteristic peaks at approximately 5.9, 8.8, 10.4, 12.3, 14.6, 17.1, 17.5, and 19.4 degrees 2θ.

In some embodiments, the arginine salt crystalline form exhibits an X-ray powder diffraction pattern comprising at least one characteristic peak, wherein said characteristic peak is selected from the group consisting of approximately 5.7, 8.6, 10.3, 10.5, 12.1, 12.4, 14.2, 14.5, 16.6, 16.9, 17.8, 19.3, 20.0, 20.5, 25.1, and 25.6 degrees 2θ.

In some embodiments, the arginine salt crystalline form exhibits an X-ray diffraction powder diffraction pattern comprising at least three characteristic peaks, wherein said characteristic peaks are selected from a group consisting of 5.7, 8.6, 10.3, 10.5, 12.1, 12.4, 14.2, 14.5, 16.6, 16.9, 17.8, 19.3, 20.0, 20.5, 25.1, and 25.6 degrees 2θ.

In some embodiments, the arginine salt crystalline form exhibits an X-ray powder diffraction pattern comprising characteristic peaks at approximately 5.7, 8.6, 10.3, 10.5, 12.1, 12.4, 14.2, 14.5, 16.6, and 16.9 degrees 2θ.

In some embodiments, the arginine salt is anhydrous.

In some embodiments, greater than 50% by weight of the total amount of the compound of Formula (I) in the composition is the arginine salt.

In some embodiments, greater than 85% by weight of the total amount of the compound of Formula (I) in the composition is the arginine salt.

In some embodiments, greater than 90% by weight of the total amount of the compound of Formula (I) in the composition is the arginine salt.

Some embodiments provide an N-methylglucamine salt form of a compound of Formula (I):

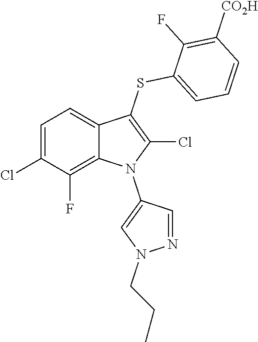 [SMILES: CCCn1cc(-n2c(Cl)c(Sc3cccc(C(=O)O)c3F)c3ccc(Cl)c(F)c32)cn1]

(I), or a solvate thereof.

In some embodiments, the N-methylglucamine salt in crystalline form.

In some embodiments, the N-methylglucamine salt crystalline form exhibits an X-ray powder diffraction pattern comprising at least one characteristic peak, wherein said characteristic peak is selected from the group consisting of approximately 8.3, 9.5, 10.5, 10.8, 12.2, 12.5, 14.2, 14.5, 16.4, 16.8, 18.4, 18.6, 19.0, 20.3, and 25.4 degrees 2θ.

In some embodiments, the N-methylglucamine salt crystalline form exhibits an X-ray diffraction powder diffraction pattern comprising at least three characteristic peaks, wherein said characteristic peaks are selected from a group consisting of approximately 8.3, 9.5, 10.5, 10.8, 12.2, 12.5, 14.2, 14.5, 16.4, 16.8, 18.4, 18.6, 19.0, 20.3, and 25.4 degrees 2θ.

In some embodiments, the N-methylglucamine salt crystalline form exhibits an X-ray powder diffraction pattern comprising characteristic peaks at approximately 8.3, 9.5, 10.5, 10.8, 12.2, 12.5, 14.2, 14.5, 16.4, 16.8, and 18.6 degrees 2θ.

In some embodiments, the N-methylglucamine salt crystalline form exhibits an X-ray powder diffraction pattern comprising at least one characteristic peak, wherein said characteristic peak is selected from the group consisting of approximately 8.5, 9.6, 11.6, 14.1, 16.1, 16.6, 19.1, 19.4, 19.7, 24.2, and 25.4 degrees 2θ.

In some embodiments, the N-methylglucamine salt crystalline form exhibits an X-ray diffraction powder diffraction pattern comprising at least three characteristic peaks, wherein said characteristic peaks are selected from a group consisting of approximately 8.5, 9.6, 11.6, 14.1, 16.1, 16.6, 19.1, 19.4, 19.7, 24.2, and 25.4 degrees 2θ.

In some embodiments, the N-methylglucamine salt crystalline form exhibits an X-ray powder diffraction pattern comprising characteristic peaks at approximately 8.5, 9.6, 11.6, 16.1, 16.6, 19.1, 19.4, 24.2, and 25.4 degrees 2θ.

In some embodiments, the N-methylglucamine salt is anhydrous.

In some embodiments, greater than 50% by weight of the total amount of the compound of Formula (I) in the composition is the N-methylglucamine salt.

In some embodiments, greater than 85% by weight of the total amount of the compound of Formula (I) in the composition is the N-methylglucamine salt.

In some embodiments, greater than 90% by weight of the total amount of the compound of Formula (I) in the composition is the N-methylglucamine salt.

Some embodiments provide a pharmaceutical composition comprising the composition and a pharmaceutically acceptable excipient.

Some embodiments provide a method of treating fibrosis, comprising administering the pharmaceutical composition to a subject in need thereof.

### BRIEF DESCRIPTION OF THE DRAWINGS

FIG. 1 is an X-ray powder diffraction pattern of crystalline Form A of Compound 1.

FIG. 2 is a differential scanning calorimetry and thermogravimetric analysis overlay for crystalline Form A of compound of Compound 1.

FIG. 3 is an X-ray powder diffraction pattern of crystalline Form B of Compound 1.

FIG. 4 is a differential scanning calorimetry and thermogravimetric analysis overlay for crystalline Form B of compound of Compound 1.

FIG. 5 is an X-ray powder diffraction pattern of crystalline Form C of Compound 1.

FIG. 6 is a differential scanning calorimetry and thermogravimetric analysis overlay for crystalline Form C of compound of Compound 1.

FIG. 7 is an X-ray powder diffraction pattern of crystalline Form D of Compound 1.

FIG. 8 is a differential scanning calorimetry and thermogravimetric analysis overlay for crystalline Form D of compound of Compound 1.

FIG. 9 is an X-ray powder diffraction pattern of crystalline Form E of Compound 1.

FIG. 10 is an X-ray powder diffraction pattern of Form 1 of Compound 1-A.

FIG. 11 is a differential scanning calorimetry and thermogravimetric analysis overlay for Form 1 of Compound 1-A.

FIG. 12 is a plot of vapor sorption/desorption curves for Form 1 of Compound 1-A.

FIG. 13 is an X-ray powder diffraction pattern of Form 2 of Compound 1-A.

FIG. 14 is a differential scanning calorimetry and thermogravimetric analysis overlay for Form 2 of Compound 1-A.

FIG. 15 is an X-ray powder diffraction pattern of Form 3 of Compound 1-B.

FIG. 16 is a differential scanning calorimetry and thermogravimetric analysis overlay for Form 3 of Compound 1-B.

FIG. 17 is a plot of vapor sorption/desorption curves for Form 3 of Compound 1-B.

FIG. 18 is an X-ray powder diffraction pattern of Form 4 of Compound 1-B.

FIG. 19 is a differential scanning calorimetry and thermogravimetric analysis overlay for Form 4 of Compound 1-B.

### DETAILED DESCRIPTION

Disclosed herein are salts and crystalline forms of a compound of Formula (I), or hydrates or solvates thereof, and methods of crystallizing the compound of Formula (I). Formula (I) is shown below:

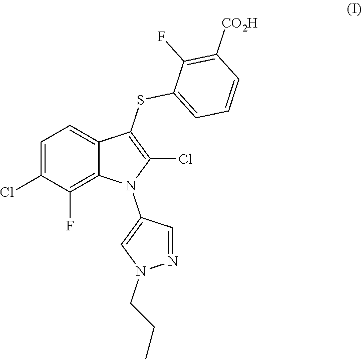 [SMILES: CCCn1cc(-n2c(Cl)c(Sc3cccc(C(=O)O)c3F)c3ccc(Cl)c(F)c32)cn1]

The compound of Formula (I) is also referred to as Compound 1 herein.

Crystalline forms of the compound of Formula (I) include Forms A, B, C, D, and E, as described below.

Also disclosed herein are various salts of Formula (I) and polymorphic forms of these salts. Salts described herein include arginine, N-methylgucamine, sodium, potassium, choline, magnesium, lysine, ammonium, betaine, TMA, proline, DEA, zinc, and calcium salts of Formula (I). Some embodiments include these salts in crystalline, partially crystalline, or non-crystalline forms.

One embodiment includes the arginine salt of the compound of Formula (I), referred to herein as Compound 1-A.

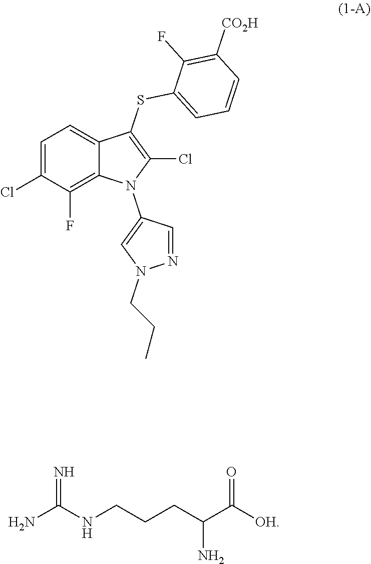 [SMILES: CCCn1cc(-n2c(Cl)c(Sc3cccc(C(=O)O)c3F)c3ccc(Cl)c(F)c32)cn1 ; N=C(N)NCCCC(N)C(=O)O]

Also disclosed herein are crystalline forms of the arginine salt of the compound of Formula (I).

Crystalline forms of Compound 1-A include Forms 1 and 2, as described below.

Further disclosed herein is the N-methylglucamine salt of the compound of formula (I), referred to herein as Compound 1-B.

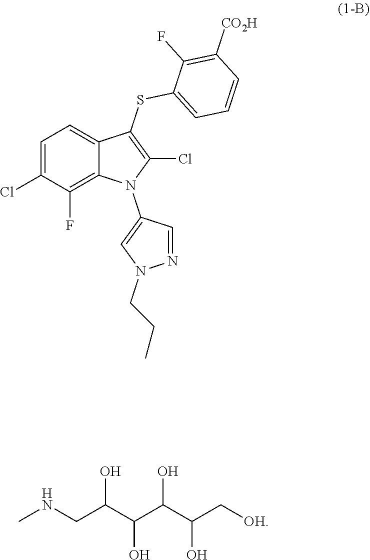 [SMILES: CCCn1cc(-n2c(Cl)c(Sc3cccc(C(=O)O)c3F)c3ccc(Cl)c(F)c32)cn1 ; CNCC(O)C(O)C(O)C(O)CO]

Also disclosed herein are crystalline forms of the N-methylglucamine salt of the compound of formula (I).

Crystalline forms of Compound 1-B include Forms 3 and 4, as described below.

In various embodiments, the salts and crystalline forms provided herein advantageously exhibit improved solubility, bioavailability, stability, processability and/or ease of manufacture. Accordingly, the crystalline forms provide significant clinical improvements as autotaxin inhibitors.

The present application also relates to the method using the crystalline forms described herein, for treating diseases and disorders by administering to a patient a therapeutically effective amount of a composition comprising one or more crystalline forms of Compound 1, Compound 1-A, and/or Compound 1-B, and one or more pharmaceutically acceptable excipients.

### Crystalline Forms of Compound 1

Disclosed herein are crystalline forms of Compound 1 and in particular crystalline Form A, crystalline Form B, crystalline Form C, crystalline Form D, and crystalline Form E, as described elsewhere herein. Unless otherwise stated, the X-ray powder diffraction data provided herein was determined using a Cu Kα radiation source.

### Crystalline Form A

Some embodiments include a solvated crystalline form of Compound 1, referred to herein as crystalline Form A. The precise conditions for forming crystalline Form A may be empirically determined and it is only possible to give a number of methods which have been found to be suitable in practice.

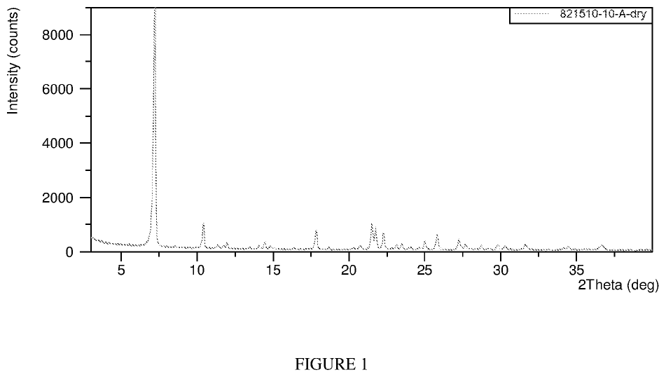

Crystalline Form A was characterized using various techniques which are described in further detail in the experimental methods section. FIG. 1 shows the crystalline structure of Form A as determined by X-ray powder diffraction (XRPD). Crystalline Form A, which may be obtained by the methods disclosed herein, exhibits characteristic peaks at approximately 7.2, 10.4, 12.0, 14.5, 17.9, 21.5, 21.8, 22.3, 23.5, 25.0, 25.8, 27.2, 27.7, 29.8, 31.6, and 36.7 degrees 2θ. Thus, in some embodiments, a crystalline form of Compound 1 has at least one characteristic peak (e.g., one, two, three, four, five, six, seven, eight, nine, ten, eleven, twelve, thirteen, fourteen, or fifteen characteristic peaks) selected from approximately 7.2, 10.4, 12.0, 14.5, 17.9, 21.5, 21.8, 22.3, 23.5, 25.0, 25.8, 27.2, 27.7, 29.8, 31.6, and 36.7 degrees 2θ. In some embodiments, a crystalline form of Compound 1 has at least three characteristic peaks selected from approximately 7.2, 10.4, 12.0, 14.5, 17.9, 21.5, 21.8, 22.3, 23.5, 25.0, 25.8, 27.2, 27.7, 29.8, 31.6, and 36.7 degrees 2θ. In some embodiments, a crystalline form of Compound 1 exhibits prominent peaks at approximately 7.2, 10.4, 12.0, 17.9, 21.5, 21.8, and 25.8, 27.2, 27.7, 29.8, 31.6, and 36.7 degrees 2θ.

As is well understood in the art, because of the experimental variability when X-ray diffraction patterns are measured on different instruments, the peak positions are assumed to be equal if the two theta (2θ) values agree to within a certain degree of variability. For example, the United States Pharmacopeia states that if the angular setting of the 10 strongest diffraction peaks agree to within ±0.2 degrees with that of a reference material, and the relative intensities of the peaks do not vary by more than 20%, the identity is confirmed. Accordingly, in some embodiments, peak positions recited herein include variability within ±0.5 degrees 2θ. In other embodiments, peak positions recited herein include variability within ±0.2 degrees 2θ. As disclosed herein, the term “approximately” when referring to values of 2θ is defined as ±0.5 degrees 2θ.

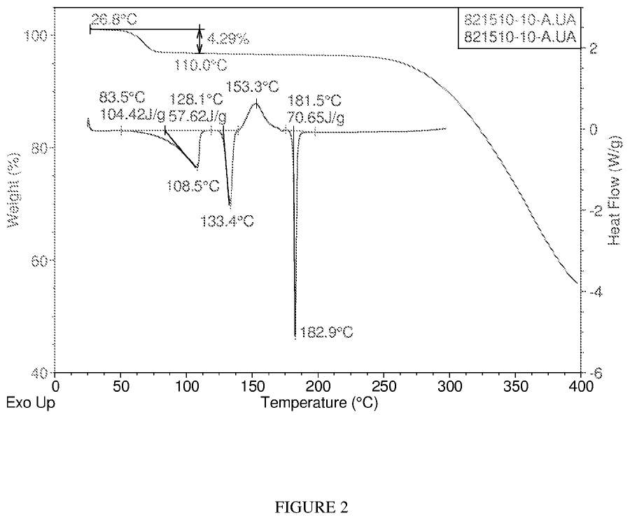

FIG. 2 shows results obtained by differential scanning calorimetry (DSC) and thermogravimetric analysis for crystalline Form A. The DSC results indicates endotherms at about 109° C., about 133° C. and about 183° C. and an exotherm at about 153° C. The DSC results indicate a peak at a temperature of about 183° C. for Crystalline Form A, which indicates the melting point for the crystal. Accordingly, in some embodiments, Crystalline Form A exhibits a melting point from about 177° C. to 200° C., from about 180° C. to about 195° C., or at about 183° C. Crystalline Form A was analysed by thermogravimetric gravimetric analysis (TG), and in one instance exhibited a 4.29% weight loss when carried out from 27° C. to 110° C.

Crystalline Form A shows good crystallinity and its melting point is relatively high (approx. 183° C.). Crystalline Form A shows evidence of hydrate formation.

### Crystalline Form B

Some embodiments include a solvated crystalline form of Compound 1, referred to herein as crystalline Form B. The precise conditions for forming crystalline Form B may be empirically determined and it is only possible to give a number of methods which have been found to be suitable in practice.

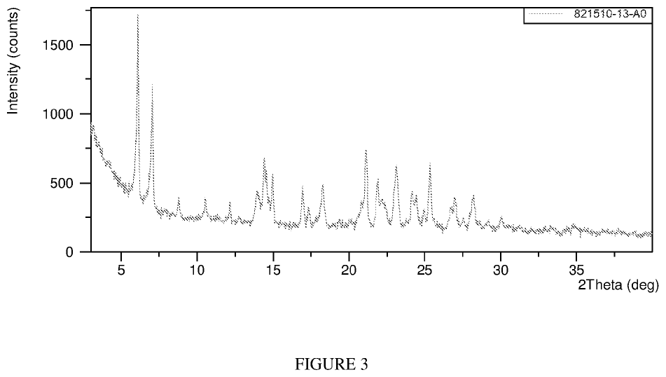

Crystalline Form B was characterized using various techniques which are described in further detail in the experimental methods section. FIG. 3 shows the crystalline structure of Form B as determined by X-ray powder diffraction (XRPD). Crystalline Form B, which may be obtained by the methods disclosed herein, exhibits characteristic peaks at approximately 6.1, 7.1, 8.8, 10.5, 12.1, 14.0, 14.4, 15.0, 17.0, 18.3, 21.1, 21.9, 23.1, 24.1, 24.4, 25.4, 27.0, 27.5, and 28.2 degrees 2θ. Thus, in some embodiments, a crystalline form of Compound 1 has at least one characteristic peak (e.g., one, two, three, four, five, six, seven, eight, nine, ten, eleven, twelve, or thirteen characteristic peaks) selected from approximately 6.1, 7.1, 8.8, 10.5, 12.1, 14.0, 14.4, 15.0, 17.0, 18.3, 21.1, 21.9, 23.1, 24.1, 24.4, 25.4, 27.0, 27.5, and 28.2 degrees 2θ. In some embodiments, a crystalline form of Compound 1 has at least three characteristic peaks selected from approximately 6.1, 7.1, 8.8, 10.5, 12.1, 14.0, 14.4, 15.0, 17.0, 18.3, 21.1, 21.9, 23.1, 24.1, 24.4, 25.4, 27.0, 27.5, and 28.2 degrees 2θ. In some embodiments, a crystalline form of Compound 1 exhibits prominent peaks at approximately 6.1, 7.1, 8.8, 14.4, 17.0, 18.3, 21.1, 21.9, 23.1, 25.4, and 28.2 degrees 2θ.

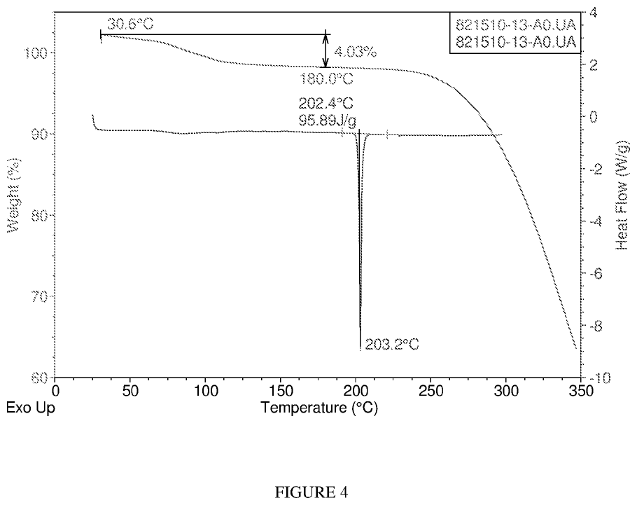

FIG. 4 shows results obtained by differential scanning calorimetry (DSC) and thermogravimetric analysis for crystalline Form B. The DSC results indicate a peak at a temperature of about 203° C. for Crystalline Form B, which indicates the melting point for the crystal. Accordingly, in some embodiments, Crystalline Form B exhibits a melting point from about 191° C. to 222° C., from about 199° C. to about 212° C., or at about 203° C. Crystalline Form B was analysed by thermogravimetric gravimetric analysis (TG), and in one instance exhibited a 4.03% weight loss when carried out from 31° C. to 180° C.

Crystalline Form B shows good crystallinity and melting point is relatively high (approx. 203° C.). Crystalline Form B shows evidence of solvate formation and is likely a solvate of ethyl alcohol.

### Crystalline Form C

Some embodiments include an anhydrous crystalline form of Compound 1, referred to herein as crystalline Form C. The precise conditions for forming crystalline Form C may be empirically determined and it is only possible to give a number of methods which have been found to be suitable in practice.

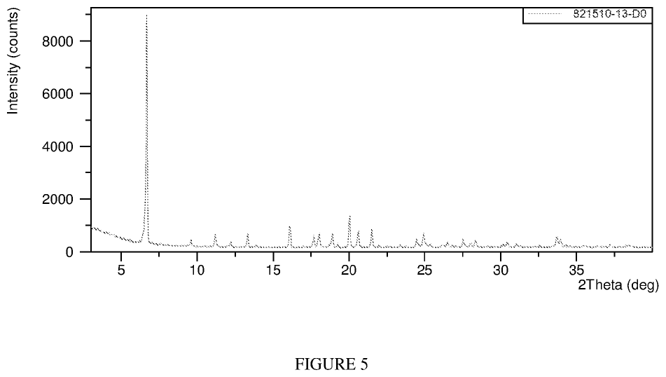

Crystalline Form C was characterized using various techniques which are described in further detail in the experimental methods section. FIG. 5 shows the crystalline structure of Form C as determined by X-ray powder diffraction (XRPD). Crystalline Form C, which may be obtained by the methods disclosed herein, exhibits characteristic peaks at approximately 6.7, 9.6, 11.2, 13.3, 16.1, 17.7, 18.1, 18.9, 19.3, 20.0, 20.6, 21.5, 24.5, 24.9, 27.5, 28.3, 33.7, and 33.9 degrees 2θ. Thus, in some embodiments, a crystalline form of Compound 1 has at least one characteristic peak (e.g., one, two, three, four, five, six, seven, eight, nine, ten, eleven, twelve, or thirteen characteristic peaks) selected from approximately 6.7, 9.6, 11.2, 13.3, 16.1, 17.7, 18.1, 18.9, 19.3, 20.0, 20.6, 21.5, 24.5, 24.9, 27.5, 28.3, 33.7, and 33.9 degrees 2θ. In some embodiments, a crystalline form of Compound 1 has at least three characteristic peaks selected from approximately 6.7, 9.6, 11.2, 13.3, 16.1, 17.7, 18.1, 18.9, 19.3, 20.0, 20.6, 21.5, 24.5, 24.9, 27.5, 28.3, 33.7, and 33.9 degrees 2θ. In some embodiments, a crystalline form of Compound 1 exhibits prominent peaks at 6.7, 11.2, 13.3, 16.1, 17.7, 18.1, 18.9, 19.3, 20.0, 20.6, 21.5, and 24.9 degrees 2θ.

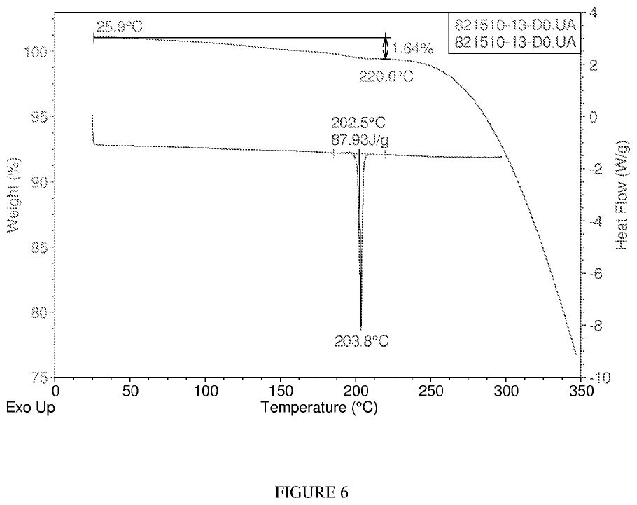

FIG. 6 shows results obtained by differential scanning calorimetry (DSC) and thermogravimetric analysis for crystalline Form C. The DSC results indicate a peak at a temperature of about 204° C. for Crystalline Form C, which indicates the melting point for the crystal. Accordingly, in some embodiments, Crystalline Form C exhibits a melting point from about 189° C. to 219° C., from about 197° C. to about 211° C., or at about 204° C. Crystalline Form C was analysed by thermogravimetric gravimetric analysis (TG), and in one instance exhibited a 1.64% weight loss when carried out from 26° C. to 220° C.

Crystalline Form C shows good crystallinity and melting point is relatively high (approx. 204° C.). Crystalline Form C does not show any evidence of hydrate formation.

### Crystalline Form D

Some embodiments include a solvated crystalline form of Compound 1, referred to herein as crystalline Form D. The precise conditions for forming crystalline Form D may be empirically determined and it is only possible to give a number of methods which have been found to be suitable in practice.

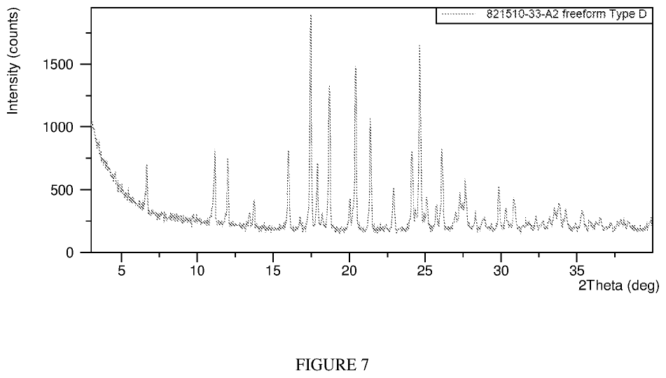

Crystalline Form D was characterized using various techniques which are described in further detail in the experimental methods section. FIG. 7 shows the crystalline structure of Form D as determined by X-ray powder diffraction (XRPD). Crystalline Form D, which may be obtained by the methods disclosed herein, exhibits characteristic peaks at approximately 6.7, 11.2, 12.0, 13.7, 16.0, 17.5, 17.9, 18.7, 20.0, 20.4, 21.4, 22.9, 24.1, 24.7, 25.1, 25.7, 26.1, 27.3, 27.7, 28.3, 28.8, 29.9, 30.3, 30.8, 33.8, 34.3, 35.4, and 38.1 degrees 2θ. Thus, in some embodiments, a crystalline form of Compound 1 has at least one characteristic peak (e.g., one, two, three, four, five, six, seven, eight, nine, ten, eleven, twelve, thirteen, fourteen, fifteen, or sixteen characteristic peaks) selected from approximately 6.7, 11.2, 12.0, 13.7, 16.0, 17.5, 17.9, 18.7, 20.0, 20.4, 21.4, 22.9, 24.1, 24.7, 25.1, 25.7, 26.1, 27.3, 27.7, 28.3, 28.8, 29.9, 30.3, 30.8, 33.8, 34.3, 35.4, and 38.1 degrees 2θ. In some embodiments, a crystalline form of Compound 1 has at least three characteristic peaks selected from approximately 6.7, 11.2, 12.0, 13.7, 16.0, 17.5, 17.9, 18.7, 20.0, 20.4, 21.4, 22.9, 24.1, 24.7, 25.1, 25.7, 26.1, 27.3, 27.7, 28.3, 28.8, 29.9, 30.3, 30.8, 33.8, 34.3, 35.4, and 38.1 degrees 2θ. In some embodiments, a crystalline form of Compound 1 exhibits prominent peaks at 6.7, 11.2, 12.0, 13.7, 16.0, 17.5, 17.9, 18.7, 20.4, 21.4, 22.9, 24.1, 24.7, 29.9, 30.3, and 30.8 degrees 2θ.

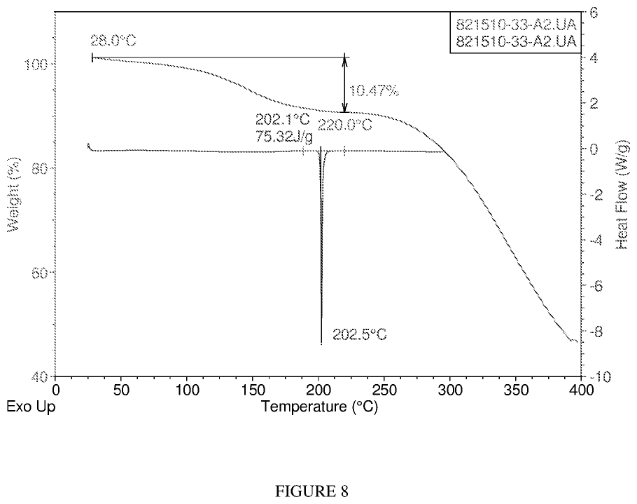

FIG. 8 shows results obtained by differential scanning calorimetry (DSC) and thermogravimetric analysis for crystalline Form D. The DSC results indicate a peak at a temperature of about 203° C. for Crystalline Form D, which indicates the melting point for the crystal. Accordingly, in some embodiments, Crystalline Form D exhibits a melting point from about 189° C. to 220° C., from about 196° C. to about 211° C., or at about 203° C. Crystalline Form D was analysed by thermogravimetric gravimetric analysis (TG), and in one instance exhibited a 10.47% weight loss when carried out from 28° C. to 220° C.

Crystalline Form D shows good crystallinity and melting point is relatively high (approx. 203° C.). Crystalline Form D shows evidence of solvate formation and is likely a solvate of chloroform.

### Crystalline Form E

Some embodiments include a crystalline form of Compound 1, referred to herein as crystalline Form E. The precise conditions for forming crystalline Form E may be empirically determined and it is only possible to give a number of methods which have been found to be suitable in practice.

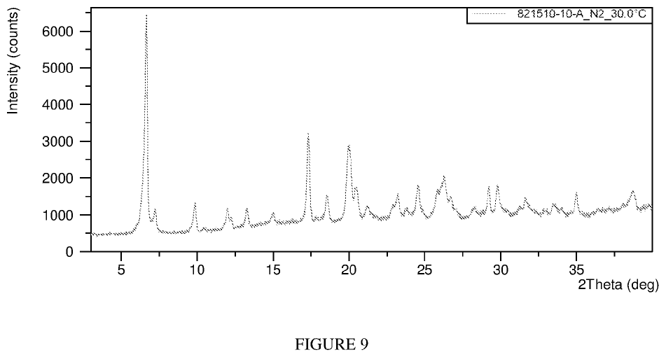

Crystalline Form E was characterized using various techniques which are described in further detail in the experimental methods section. FIG. 9 shows the crystalline structure of Form E as determined by X-ray powder diffraction (XRPD). Crystalline Form E, which may be obtained by the methods disclosed herein, exhibits characteristic peaks at approximately 6.7, 7.3, 9.9, 12.0, 13.3, 15.0, 17.3, 18.6, 19.9, 20.5, 21.3, 23.2, 23.8, 24.6, 25.8, 26.3, 26.7, 28.3, 29.2, 29.8, 31.6, 33.5, 35.0, and 38.7 degrees 2θ. Thus, in some embodiments, a crystalline form of Compound 1 has at least one characteristic peak (e.g., one, two, three, four, five, six, seven, eight, nine, ten, or eleven characteristic peaks) selected from approximately 6.7, 7.3, 9.9, 12.0, 13.3, 15.0, 17.3, 18.6, 19.9, 20.5, 21.3, 23.2, 23.8, 24.6, 25.8, 26.3, 26.7, 28.3, 29.2, 29.8, 31.6, 33.5, 35.0, and 38.7 degrees 2θ. In some embodiments, a crystalline form of Compound 1 has at least three characteristic peaks selected from approximately 6.7, 7.3, 9.9, 12.0, 13.3, 15.0, 17.3, 18.6, 19.9, 20.5, 21.3, 23.2, 23.8, 24.6, 25.8, 26.3, 26.7, 28.3, 29.2, 29.8, 31.6, 33.5, 35.0, and 38.7 degrees 2θ. In some embodiments, a crystalline form of Compound 1 exhibits prominent peaks at 6.7, 9.9, 12.0, 13.3, 17.3, 18.6, 19.9, 23.2, 24.6, 26.3, 29.2, 29.8, 35.0, and 38.7 degrees 2θ.

In some embodiments, the crystalline form of Compound 1 is a hydrate. In some embodiments, crystalline form A is a hydrate. In some embodiments, crystalline form E is a hydrate.

In some embodiments, the crystalline form of Compound 1 is anhydrous. In some embodiments, crystalline form C is anhydrous. In some embodiments, crystalline form E is anhydrous.

In some embodiments, the crystalline form of Compound 1 is unsolvated. In some embodiments, crystalline form C is unsolvated. In some embodiments, crystalline form E is unsolvated.

In some embodiments, the crystalline form of Compound 1 is a solvate. In some embodiments, crystalline form B is a solvate. In some embodiments, crystalline form C is a solvate. In some embodiments, crystalline form D is a solvate. In some embodiments, crystalline form E is a solvate.

In some embodiments, the crystalline form of Compound 1 is an ethanol solvate. In some embodiments, crystalline form B is an ethanol solvate. In some embodiments, crystalline form E is an ethanol solvate.

In some embodiments, the crystalline form of Compound 1 is a chloroform solvate. In some embodiments, crystalline form D is a chloroform solvate. In some embodiments, crystalline form E is a chloroform solvate.

Some embodiments of the present disclosure include compositions of a compound of Formula (I), or hydrates or solvates thereof. In some embodiments, the compositions comprise one or more crystalline forms of Compound 1. In some embodiments, the compositions comprise crystalline form A, crystalline form B, crystalline form C, crystalline form D, crystalline form E, and any combination of the foregoing. In some embodiments, the compositions comprise crystalline form A. In some embodiments, the compositions comprise crystalline form B. In some embodiments, the compositions comprise crystalline form C. In some embodiments, the compositions comprise crystalline form D. In some embodiments, the compositions comprise crystalline form E. In some embodiments, greater than 50% by weight of the total amount of Compound 1 in the composition is the crystalline form. In some embodiments, greater than 85% by weight of the total amount of Compound 1 in the composition is the crystalline form. In some embodiments, greater than 90% by weight of the total amount of Compound 1 in the composition is the crystalline form.

### Compound 1-A and Crystalline Forms Thereof

Disclosed herein is Compound 1-A, which is the arginine salt form of Compound 1. The structure of Compound 1-A is provided below.

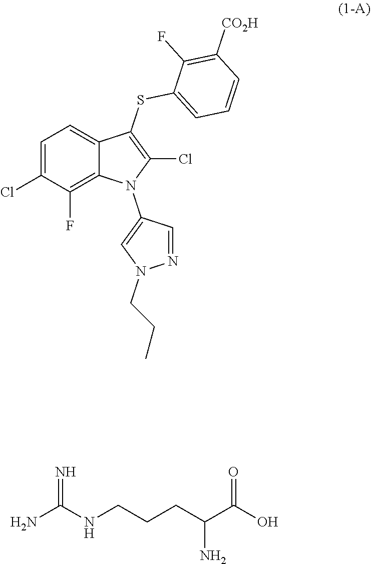 [SMILES: CCCn1cc(-n2c(Cl)c(Sc3cccc(C(=O)O)c3F)c3ccc(Cl)c(F)c32)cn1 ; N=C(N)NCCCC(N)C(=O)O]

Also disclosed herein are crystalline forms of Compound 1-A, and in particular crystalline Form 1 and crystalline Form 2. (described below). Unless otherwise stated, the X-ray powder diffraction data provided herein was determined using a Cu Kα radiation source.

### Crystalline Form 1

Some embodiments include an unsolvated crystalline form of Compound 1-A, referred to herein as crystalline Form 1. The precise conditions for forming crystalline Form 1 may be empirically determined and it is only possible to give a number of methods which have been found to be suitable in practice.

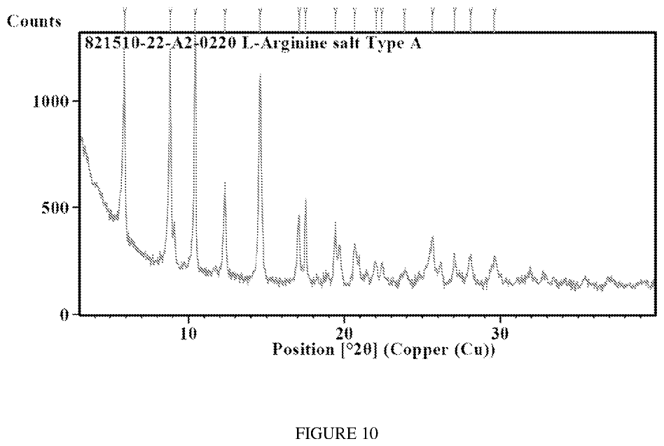

Crystalline Form 1 was characterized using various techniques which are described in further detail in the experimental methods section. FIG. 10 shows the crystalline structure of Form 1 as determined by X-ray powder diffraction (XRPD). Crystalline Form 1, which may be obtained by the methods disclosed herein, exhibits characteristic peaks at approximately 5.9, 8.8, 10.4, 12.3, 14.6, 17.1, 17.5, 19.4, 20.7, 25.7, 27.0, and 28.1 degrees 2θ. Thus, in some embodiments, a crystalline form of Compound 1-A has at least one characteristic peak (e.g., one, two, three, four, five, six, seven, eight, nine, ten, eleven, twelve, thirteen, fourteen, fifteen, or sixteen characteristic peaks) selected from approximately 5.9, 8.8, 10.4, 12.3, 14.6, 17.1, 17.5, 19.4, 20.7, 25.7, 27.0, and 28.1 degrees 2θ. In some embodiments, a crystalline form of Compound 1-A has at least three characteristic peaks selected from approximately 5.9, 8.8, 10.4, 12.3, 14.6, 17.1, 17.5, 19.4, 20.7, 25.7, 27.0, and 28.1 degrees 2θ. In some embodiments, a crystalline form of Compound 1-A exhibits prominent peaks at 5.9, 8.8, 10.4, 12.3, 14.6, 17.1, 17.5, and 19.4 degrees 2θ.

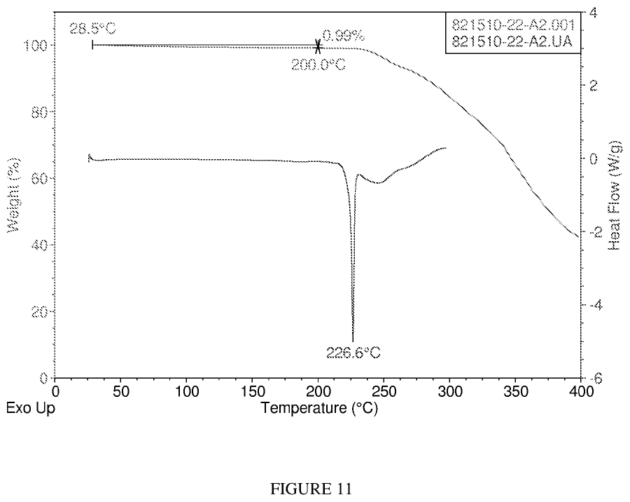

FIG. 11 shows results obtained by differential scanning calorimetry (DSC) and thermogravimetric analysis for crystalline Form 1. The DSC results indicate a peak at a temperature of about 227° C. for crystalline Form 1, which indicates the melting point for the crystal. Accordingly, in some embodiments, crystalline Form 1 exhibits a melting point from about 210° C. to 245° C., from about 218° C. to about 235° C., or at about 227° C. 1H-NMR results are consistent with a 1:1 molar salt of Compound 1 and L-arginine. Crystalline Form 1 was analysed by thermogravimetric gravimetric analysis (TG), and in one instance exhibited a 1.0% weight loss when carried out from 29° C. to 200° C.

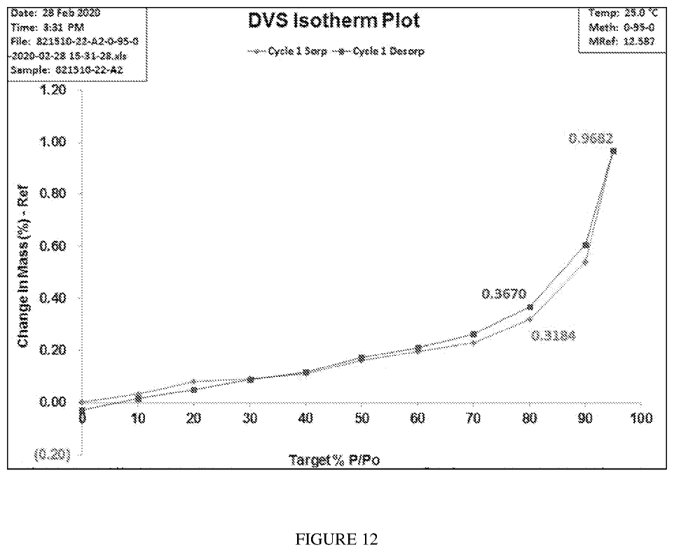

FIG. 12 shows results obtained by Dynamic Vapor Sorption (DVS) indicating that crystalline Form 1 showed a moisture uptake of 0.32% at 25° C./80% RH, indicating that crystalline Form 1 was slightly hygroscopic. Crystalline Form 1 can therefore be characterized as slightly hygroscopic and stable over a wide range of humidity.

Crystalline Form 1 shows good crystallinity and melting point is relatively high (approx. 227° C.) and does not show any evidence of hydrate formation.

### Crystalline Form 2

Some embodiments include a crystalline form of Compound 1-A, referred to herein as crystalline Form 2. The precise conditions for forming crystalline Form 2 may be empirically determined and it is only possible to give a number of methods which have been found to be suitable in practice.

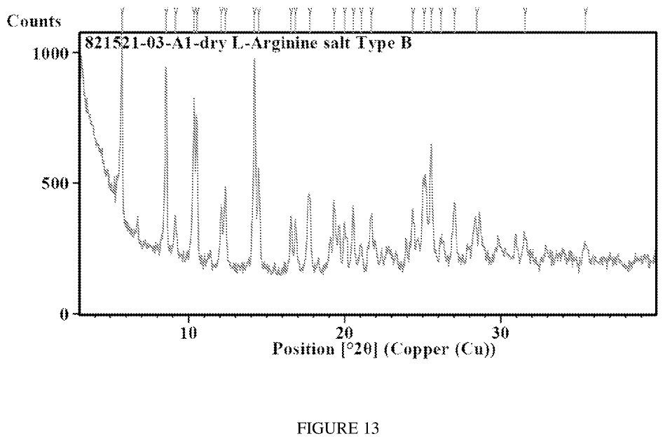

Crystalline Form 2 was characterized using various techniques which are described in further detail in the experimental methods section. FIG. 13 shows the crystalline structure of Form 2 as determined by X-ray powder diffraction (XRPD). Crystalline Form 2, which may be obtained by the methods disclosed herein, exhibits characteristic peaks at approximately 5.7, 8.6, 10.3, 10.5, 12.1, 12.4, 14.2, 14.5, 16.6, 16.9, 17.8, 19.3, 20.0, 20.5, 25.1, and 25.6 degrees 2θ. Thus, in some embodiments, a crystalline form of Compound 1-A has at least one characteristic peak (e.g., one, two, three, four, five, six, seven, eight, nine, ten, or eleven characteristic peaks) selected from approximately 5.7, 8.6, 10.3, 10.5, 12.1, 12.4, 14.2, 14.5, 16.6, 16.9, 17.8, 19.3, 20.0, 20.5, 25.1, and 25.6 degrees 2θ. In some embodiments, a crystalline form of Compound 1-A has at least three characteristic peaks selected from approximately 5.7, 8.6, 10.3, 10.5, 12.1, 12.4, 14.2, 14.5, 16.6, 16.9, 17.8, 19.3, 20.0, 20.5, 25.1, and 25.6 degrees 2θ. In some embodiments, a crystalline form of Compound 1-A exhibits prominent peaks at 5.7, 8.6, 10.3, 10.5, 12.1, 12.4, 14.2, 14.5, 16.6, and 16.9 degrees 2θ.

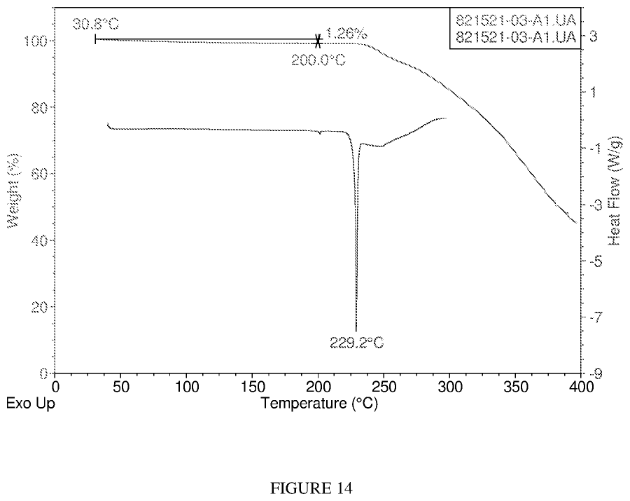

FIG. 14 shows results obtained by differential scanning calorimetry (DSC) and thermogravimetric analysis for crystalline Form 2. The DSC results indicate a peak at a temperature of about 229° C. for crystalline Form 2, which indicates the melting point for the crystal. Accordingly, in some embodiments, crystalline Form 2 exhibits a melting point from about 210° C. to 245° C., from about 218° C. to about 235° C., or at about 229° C.

In some embodiments, the crystalline form of Compound 1-A is anhydrous. In some embodiments, crystalline Form 1 is anhydrous. In some embodiments, crystalline Form 2 is anhydrous.

Some embodiments of the present disclosure include compositions of a compound of Formula (I), comprising the arginine salt form of Compound 1, (i.e., Compound 1-A). In some embodiments, greater than 50% by weight of the total amount of the compound of Formula (I) in the composition is Compound 1-A. In some embodiments, greater than 85% by weight of the total amount of the compound of Formula (I) in the composition is Compound 1-A. In some embodiments, greater than 90% by weight of the total amount of the compound of Formula (I) in the composition is Compound 1-A.

In some embodiments, the compositions comprise the crystalline form of Compound 1-A. In some embodiments, the compositions comprise crystalline Form 1, crystalline Form 2, and any combination of the foregoing. In some embodiments, the compositions comprise crystalline Form 1. In some embodiments, the compositions comprise crystalline Form 2. In some embodiments, greater than 50% by weight of the total amount of Compound 1-A in the composition is the crystalline form. In some embodiments, the compositions comprise crystalline Form 2. In some embodiments, greater than 85% by weight of the total amount of Compound 1-A in the composition is the crystalline form. In some embodiments, the compositions comprise crystalline Form 2. In some embodiments, greater than 90% by weight of the total amount of Compound 1-A in the composition is the crystalline form.

### Compound 1-B and Crystalline Forms Thereof

Disclosed herein is Compound 1-B, which is the N-methylglucamine salt form of Compound 1. The structure of Compound 1-B is provided below.

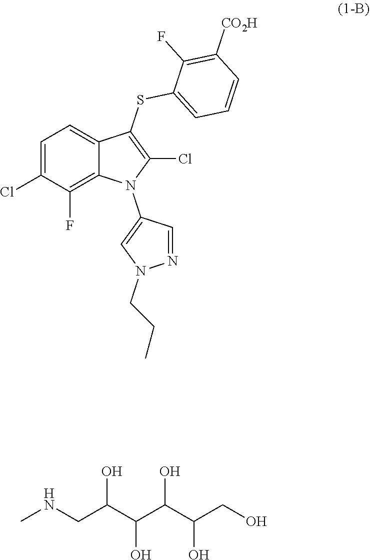 [SMILES: CCCn1cc(-n2c(Cl)c(Sc3cccc(C(=O)O)c3F)c3ccc(Cl)c(F)c32)cn1 ; CNCC(O)C(O)C(O)C(O)CO]

Also disclosed herein are crystalline forms of Compound 1-B, and in particular crystalline Form 3 and crystalline Form 4. (described below). Unless otherwise stated, the X-ray powder diffraction data provided herein was determined using a Cu Kα radiation source.

### Crystalline Form 3

Some embodiments include an unsolvated crystalline form of Compound 1-B, referred to herein as crystalline Form 3. The precise conditions for forming crystalline Form 3 may be empirically determined and it is only possible to give a number of methods which have been found to be suitable in practice.

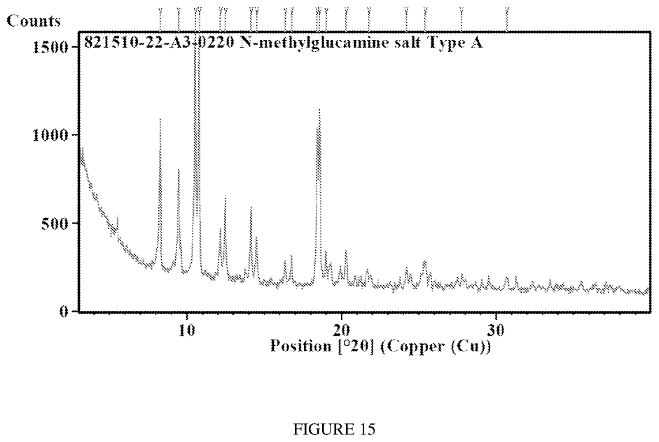

Crystalline Form 3 was characterized using various techniques which are described in further detail in the experimental methods section. FIG. 15 shows the crystalline structure of Form 3 as determined by X-ray powder diffraction (XRPD). Crystalline Form 3, which may be obtained by the methods disclosed herein, exhibits characteristic peaks at approximately 8.3, 9.5, 10.5, 10.8, 12.2, 12.5, 14.2, 14.5, 16.4, 16.8, 18.4, 18.6, 19.0, 20.3, and 25.4 degrees 2θ. Thus, in some embodiments, a crystalline form of Compound 1-B has at least one characteristic peak (e.g., one, two, three, four, five, six, seven, eight, nine, ten, eleven, twelve, thirteen, fourteen, fifteen, or sixteen characteristic peaks) selected from approximately 8.3, 9.5, 10.5, 10.8, 12.2, 12.5, 14.2, 14.5, 16.4, 16.8, 18.4, 18.6, 19.0, 20.3, and 25.4 degrees 2θ. In some embodiments, a crystalline form of Compound 1-B has at least three characteristic peaks selected from approximately 8.3, 9.5, 10.5, 10.8, 12.2, 12.5, 14.2, 14.5, 16.4, 16.8, 18.4, 18.6, 19.0, 20.3, and 25.4 degrees 2θ. In some embodiments, a crystalline form of Compound 1-B exhibits prominent peaks at 8.3, 9.5, 10.5, 10.8, 12.2, 12.5, 14.2, 14.5, 16.4, 16.8, and 18.6 degrees 2θ.

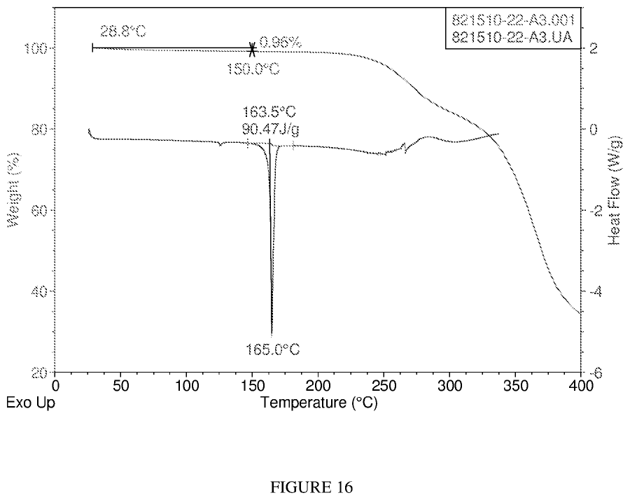

FIG. 16 shows results obtained by differential scanning calorimetry (DSC) and thermogravimetric analysis for crystalline Form 3. The DSC results indicate a peak at a temperature of about 165° C. for crystalline Form 3, which indicates the melting point for the crystal. Accordingly, in some embodiments, crystalline Form 3 exhibits a melting point from about 150° C. to 180° C., from about 157° C. to about 173° C., or at about 165° C. ¹H-NMR results are consistent with a 1:1 molar salt of Compound 1 and N-methylglucamine. Crystalline Form 3 was analysed by thermogravimetric gravimetric analysis (TG), and in one instance exhibited a 1.0% weight loss when carried out from 29° C. to 150° C.

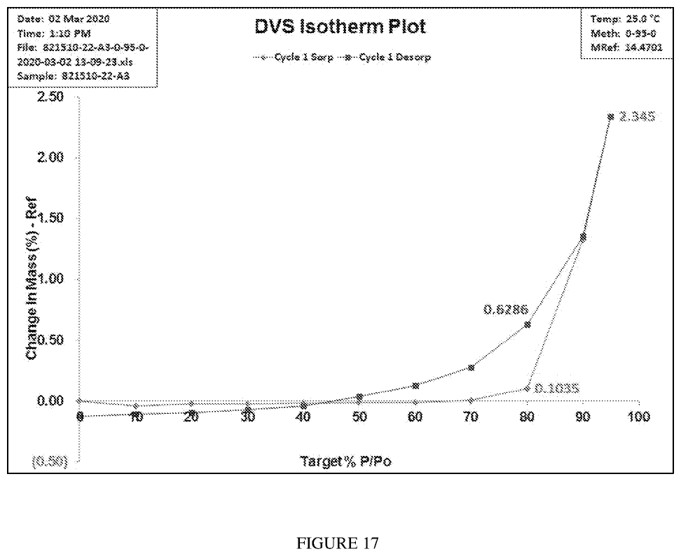

FIG. 17 shows results obtained by Dynamic Vapor Sorption (DVS) indicating that crystalline Form 1 showed a moisture uptake of 0.1% at 25° C./80% RH, indicating that crystalline Form 1 was non-hygroscopic. Crystalline Form 3 can therefore be characterized as non-hygroscopic and stable over a wide range of humidity.

Crystalline Form 3 shows good crystallinity and melting point is relatively high (approx. 165° C.) and does not show any evidence of hydrate formation.

### Crystalline Form 4

Some embodiments include a crystalline form of Compound 1-B, referred to herein as crystalline Form 4. The precise conditions for forming crystalline Form 4 may be empirically determined and it is only possible to give a number of methods which have been found to be suitable in practice.

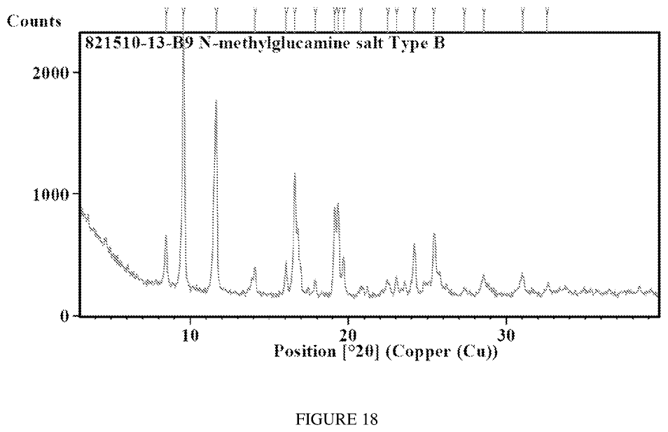

Crystalline Form 4 was characterized using various techniques which are described in further detail in the experimental methods section. FIG. 18 shows the crystalline structure of Form 4 as determined by X-ray powder diffraction (XRPD). Crystalline Form 4, which may be obtained by the methods disclosed herein, exhibits characteristic peaks at approximately 8.5, 9.6, 11.6, 14.1, 16.1, 16.6, 19.1, 19.4, 19.7, 24.2, and 25.4 degrees 2θ. Thus, in some embodiments, a crystalline form of Compound 1-B has at least one characteristic peak (e.g., one, two, three, four, five, six, seven, eight, nine, ten, or eleven characteristic peaks) selected from approximately 8.5, 9.6, 11.6, 14.1, 16.1, 16.6, 19.1, 19.4, 19.7, 24.2, and 25.4 degrees 2θ. In some embodiments, a crystalline form of Compound 1-B has at least three characteristic peaks selected from approximately 8.5, 9.6, 11.6, 14.1, 16.1, 16.6, 19.1, 19.4, 19.7, 24.2, and 25.4 degrees 2θ. In some embodiments, a crystalline form of Compound 1-B exhibits prominent peaks at 8.5, 9.6, 11.6, 16.1, 16.6, 19.1, 19.4, 24.2, and 25.4 degrees 2θ.

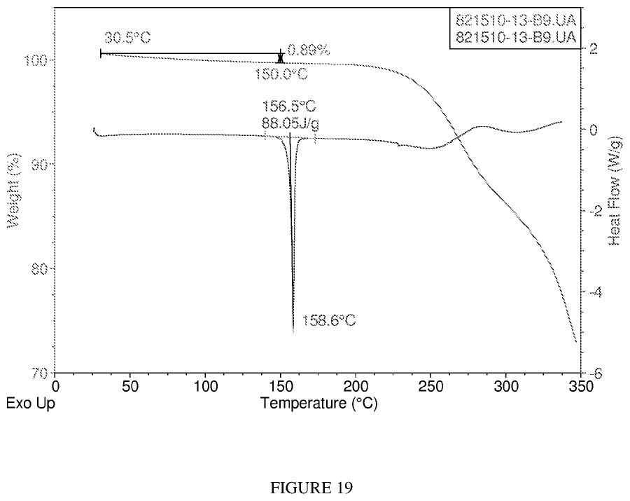

FIG. 19 shows results obtained by differential scanning calorimetry (DSC) and thermogravimetric analysis for crystalline Form 4. The DSC results indicate a peak at a temperature of about 159° C. for crystalline Form 4, which indicates the melting point for the crystal. Accordingly, in some embodiments, crystalline Form 4 exhibits a melting point from about 138° C. to 173° C., from about 147° C. to about 167° C., or at about 159° C.

In some embodiments, the crystalline form of Compound 1-B is anhydrous. In some embodiments, crystalline Form 3 is anhydrous. In some embodiments, crystalline Form 4 is anhydrous.

Some embodiments of the present disclosure include compositions of a compound of Formula (I), comprising the N-methylglucamine salt form of Compound 1, (i.e., Compound 1-B). In some embodiments, greater than 50% by weight of the total amount of the compound of Formula (I) in the composition is Compound 1-B. In some embodiments, greater than 85% by weight of the total amount of the compound of Formula (I) in the composition is Compound 1-B. In some embodiments, greater than 90% by weight of the total amount of the compound of Formula (I) in the composition is Compound 1-B.

In some embodiments, the compositions comprise the crystalline form of Compound 1-B. In some embodiments, the compositions comprise crystalline Form 3, crystalline Form 4, and any combination of the foregoing. In some embodiments, the compositions comprise crystalline Form 3. In some embodiments, the compositions comprise crystalline Form 4. In some embodiments, greater than 50% by weight of the total amount of Compound 1-B in the composition is the crystalline form. In some embodiments, the compositions comprise crystalline Form 2. In some embodiments, greater than 85% by weight of the total amount of Compound 1-B in the composition is the crystalline form. In some embodiments, the compositions comprise crystalline Form 2. In some embodiments, greater than 90% by weight of the total amount of Compound 1-B in the composition is the crystalline form.

### Methods of Crystalizing Compounds 1, 1-A, and 1-B

Disclosed are methods of crystalizing Compound 1. Crystalline forms of Compound 1 may generally be obtained or produced by crystallizing Compound 1 under controlled conditions. In some embodiments, the method may produce an unsolvated crystalline form. In some embodiments, the method may produce a solvated crystalline form. In some embodiments, the method may produce a hydrated crystalline form. In some embodiments, the method may produce crystalline Form A. In some embodiments, the method may produce crystalline Form B. In some embodiments, the method may produce crystalline Form C. In some embodiments, the method may produce crystalline Form D. In some embodiments, the method may produce crystalline Form E. In some embodiments, the method may produce a mixture of any of the aforementioned crystalline forms.

Also disclosed are methods of crystalizing Compound 1-A. Crystalline forms of Compound 1-A may generally be obtained or produced by crystallizing Compound 1-A under controlled conditions. In some embodiments, the method may produce an unsolvated crystalline form. In some embodiments, the method may produce a solvated crystalline form. In some embodiments, the method may produce a hydrated crystalline form. In some embodiments, the method may produce crystalline Form 1. In some embodiments, the method may produce crystalline Form 2. In some embodiments, the method may produce a mixture of any of the aforementioned crystalline forms.

Also disclosed are methods of crystalizing Compound 1-B. Crystalline forms of Compound 1-B may generally be obtained or produced by crystallizing Compound 1-B under controlled conditions. In some embodiments, the method may produce an unsolvated crystalline form. In some embodiments, the method may produce a solvated crystalline form. In some embodiments, the method may produce a hydrated crystalline form. In some embodiments, the method may produce crystalline Form 3. In some embodiments, the method may produce crystalline Form 4. In some embodiments, the method may produce a mixture of any of the aforementioned crystalline forms.

In some embodiments, the method may comprise dissolving an amorphous form of Compound 1, Compound 1-A, Compound 1-B, or a combination thereof in a first solvent to create a first solution. In some embodiments, the method may comprise dissolving a crystalline form of Compound 1, Compound 1-A, Compound 1-B, or a combination thereof in a first solvent to create a first solution. In some embodiments, the method may comprise dissolving a mixture of amorphous and crystalline forms of Compound 1, Compound 1-A, Compound 1-B, or a combination thereof in a first solvent to create a first solution. In some embodiments, the method may comprise adding a second solvent to the first solution to create a second mixture. In some embodiments, the method may comprise evaporating the second mixture. In some embodiments, the method may comprise cooling the second mixture. In some embodiments, the second mixture may be cooled to 25° C., 20° C., 15° C., 10° C., 5° C., 0° C., −5° C., −10° C., −20° C., −25° C., any range between any of these values, or below −25° C.

In some embodiments, the method may comprise isolating the crystalline form of Compound 1, Compound 1-A, Compound 1-B, or a combination thereof. In some embodiments, isolation may performed by filtration, such as hot-filtration. In some embodiments, the isolated product may be dried, such as by air drying.

In some embodiments, the first solvent may be a single solvent. In some embodiments, the first solvent may be a mixture of two or more solvents. In some embodiments, the first solvent may be acetone, acetonitrile, 1,4-dioxane, dimethylformamide, ethanol, ethyl acetate, diethyl ether, methanol, methyl ethyl ketone, 2-methyltetrahydrofuran, isopropanol, n-propanol, isopropyl acetate, tetrahydrofuran, water, or a mixture thereof.

In some embodiments, the second solvent may be a single solvent. In some embodiments, the second solvent may be a mixture of two or more solvents. In some embodiments, the first solvent may comprise acetone, acetonitrile, 1,4-dioxane, dichloromethane, diethyl ether, dimethylformamide, ethanol, ethyl acetate, methanol, methyl ethyl ketone, methyl isobutyl ketone, 2-methyltetrahydrofuran, isopropanol, n-propanol, isopropyl acetate, tetrahydrofuran, water, or a mixture thereof. In some embodiments, the second solvent may comprise toluene, hexanes, water, dichloromethane, or a combination thereof.

In some embodiments, the method may further comprise agitation. In some embodiments, agitation may be performed by stirring. In some embodiments, agitation may be performed by sonication.

In some embodiments, portions of the method may performed at the same temperature. In some embodiments, portions of the method may be performed at various temperatures. In some embodiments, portions of the method may be performed at room temperature. In some embodiments, portions of the method may be performed at −40° C. to 200° C. In some embodiments, portions of the method may be performed at −40° C. to 25° C. In some embodiments, portions of the method may be performed at −25° C. to −10° C. In some embodiments, portions of the method may be performed at 2° C. to 8° C. In some embodiments, portions of the method may be performed at 50° C. to 60° C. In some embodiments, portions of the method may be performed at 65° C. to 75° C. In some embodiments, portions of the method may be performed at 75° C. to 150° C. In some embodiments, portions of the method may include the first solution, second mixture, seeded mixture, isolation of the crystalline form, and agitation.

In some embodiments, a crystalline form of Compound 1 may be prepared by dissolving a quantity of Compound 1 in a minimal solvent at a first temperature to form a first solution. In some embodiments, the solvent may be acetonitrile. In other embodiments, the solvent may be dichloromethane. In yet other embodiments, the solvent may be acetone. In still yet other embodiments, the solvent may be isopropyl acetate. In other embodiments the solvent may by methyl isobutyl ketone. In some embodiments, the first temperature is from 40° C. to 60° C. or 45° C. to 55° C. In some specific embodiments, the first temperature is about 50° C. In some embodiments, the first solvent may be cooled to a second temperature. In some embodiments, the second temperature may be −5° C. to −25° C. or −10° C. to −20° C. In other embodiments, the second temperature may be room temperature. In some specific embodiments, the second temperature is about −15° C. In some embodiments, an antisolvent may be added to the first solution prior to cooling. In some embodiments, the antisolvent is selected from hexane, n-heptane, toluene, water, or a combination thereof. In some embodiments, the antisolvent is hexane. In other embodiments, the antisolvent is toluene. In other embodiments, the antisolvent is n-heptane. In other embodiments, the antisolvent is water. The method described herein can be used to prepare the crystalline Form A, the crystalline Form B, the crystalline Form C, the crystalline Form D, the crystalline Form E, or a combination of the foregoing. In some embodiments, the method described herein can be used to prepare the crystalline Form A. In some embodiments, the method described herein can be used to prepare the crystalline Form B. In some embodiments, the method described herein can be used to prepare the crystalline Form C. In some embodiments, the method described herein can be used to prepare the crystalline Form D. In some embodiments, the method described herein can be used to prepare the crystalline Form E.

In some embodiments, a crystalline form of Compound 1 may be prepared by adding Compound 1 to a solvent to form a slurry. In some embodiments, the solvent is acetone, acetonitrile, ethanol, ethyl acetate, methanol, methyl ethyl ketone, tetrahydrofuran, water or a combination thereof. In some embodiments, the solvent is acctone. In other embodiments, the solvent is water. In some embodiments, the solvent is a combination of acetone and water. In some embodiments, the solvent is a combination of methanol and water. In some embodiments, the solvent combination comprises a volume to volume (v/v) solvent ratio selected from 99/1, 95/5, 9/1, 85/15, 8/2, 7/3, 6/4, 1/1, 4/6, 3/7, 2/8, 15/85, 1/9, 5/95, or 1/99, or a range between any two of these values. In some embodiment the slurry may be stirred at 20, 25, 30, 35, 40, 45, or 50° C., or a range between any two of these values. In some embodiments, the slurry may be stirred for 1, 2, 3, 4, 5, 6, or 7 days or more. The method described herein can be used to prepare the crystalline Form A, the crystalline Form B, the crystalline Form C, the crystalline Form D, the crystalline Form E, or a combination of the foregoing. In some embodiments, the method described herein can be used to prepare the crystalline Form A. In some embodiments, the method described herein can be used to prepare the crystalline Form B. In some embodiments, the method described herein can be used to prepare the crystalline Form C. In some embodiments, the method described herein can be used to prepare the crystalline Form D. In some embodiments, the method described herein can be used to prepare the crystalline Form E.

In some embodiments, a crystalline form of Compound 1 may be prepared by adding a first crystalline form of Compound 1 to a solvent to form a saturated solution followed by addition to the saturated solution equal parts of the first crystalline form of Compound 1 and a second crystalline form of Compound 1. In some embodiments, the solvent is acetone, acetonitrile, ethanol, ethyl acetate, methanol, methyl ethyl ketone, tetrahydrofuran, water or a combination thereof. In some embodiments, the solvent is acetone. In other embodiments, the solvent is water. In some embodiments, the solvent is a combination of acetone and water. In some embodiments, the solvent is a combination of methanol and water. In some embodiments, the solvent combination comprises a volume to volume (v/v) solvent ratio selected from 99/1, 95/5, 9/1, 85/15, 8/2, 7/3, 6/4, 1/1, 4/6, 3/7, 2/8, 15/85, 1/9, 5/95, or 1/99, or a range between any two of these values. In some embodiments, the saturated solution may be stirred at 20, 25, 30, 35, 40, 45, or 50° C., or a range between any two of these values. In some embodiments, the saturated solution may be stirred for 1, 2, 3, 4, 5, 6, or 7 days or more. In some embodiments, the first crystalline form of Compound 1 is selected from crystalline Form A, crystalline Form B, crystalline Form C, crystalline Form D, or crystalline Form E. In some embodiments, the second crystalline form of Compound 1 is selected from crystalline Form A, crystalline Form B, crystalline Form C, crystalline Form D, or crystalline Form E. The method described herein can be used to prepare the crystalline Form A, the crystalline Form B, the crystalline Form C, the crystalline Form D, the crystalline Form E, or a combination of the foregoing. In some embodiments, the method described herein can be used to prepare the crystalline Form A. In some embodiments, the method described herein can be used to prepare the crystalline Form B. In some embodiments, the method described herein can be used to prepare the crystalline Form C. In some embodiments, the method described herein can be used to prepare the crystalline Form D. In some embodiments, the method described herein can be used to prepare the crystalline Form E.

In some embodiments, a crystalline form of Compound 1-A may be prepared by combining Compound 1 with a solvent and an amount of arginine to form a suspension. In some embodiments, the arginine can be a single enantiomer. In some embodiments, the arginine can be L-arginine. In other embodiments, the arginine can be D-arginine. In some embodiments, the amount of arginine is sufficient to provide a molar ratio of arginine to Compound 1 that is 1:1, 1.05:1, 1.1:1, 1.2:1, 1.3:1, 1.4:1, 1.5:1, 2:1, 3:1, or a range between any two of these values. In some embodiments, the molar ratio of arginine to Compound 1 is from 1:1 to 1.1:1. In some specific embodiments, molar ratio of arginine to Compound 1 is from 1.0:1.0 to 1.05:1.0. In some specific embodiments, molar ratio of arginine to Compound 1 is 1.0:1.0. The suspension may be stirred at room temperature for 1, 2, 3, 4, 5, or more days. In some embodiments, the suspension may be sonicated prior to addition of arginine. In some embodiments, the solvent is acetone. In other embodiments, the solvent is acetonitrile. In other embodiments, the solvent is 1,4-dioxane. In yet other embodiments, the solvent is diethyl ether. In some embodiments, the solvent is isopropanol. In other embodiments, the solvent is tetrahydrofuran. In some embodiments, the solvent is methyl ethyl ketone. In some embodiments, the solvent is ethanol. In other embodiments, the solvent is water. In some embodiments, the solvent is a combination of acetone and water. In some embodiments, the solvent is a combination of methanol and water. In some embodiments, the solvent combination comprises a volume to volume (v/v) solvent ratio selected from 99/1, 95/5, 9/1, 85/15, 8/2, 7/3, 6/4, 1/1, 4/6, 3/7, 2/8, 15/85, 1/9, 5/95, or 1/99, or a range between any two of these values. In some embodiments, the solvent is a combination of acetone/water (95:5 v/v). The method described herein can be used to prepare crystalline Form 1, crystalline Form 2, or a combination of the foregoing. In some embodiments, the method is used to prepare crystalline Form 1. In some embodiments, the method is used to prepare crystalline Form 2.

In some embodiments, a crystalline form of Compound 1-A may be prepared by combining Compound 1 with a first solvent and an amount of arginine to form a first mixture. In some embodiments, the arginine can be a single enantiomer. In some embodiments, the arginine can be L-arginine. In other embodiments, the arginine can be D-arginine. In some embodiments, the amount of arginine is sufficient to provide a molar ratio of arginine to Compound 1 that is 1:1, 1.05:1, 1.1:1, 1.2:1, 1.3:1, 1.4:1, 1.5:1, 2:1, 3:1, or a range between any two of these values. In some embodiments, the molar ratio of arginine to Compound 1 is from 1:1 to 1.1:1. In some specific embodiments, molar ratio of arginine to Compound 1 is from 1.0:1.0 to 1.05:1.0. In some specific embodiments, molar ratio of arginine to Compound 1 is 1.0:1.0. In some embodiments, the first solvent is ethanol. In some embodiments, the first solvent is acetone. In some embodiments, the first solvent is ethyl acetate. In some embodiments, the first solvent is acetonitrile. In other embodiments, the first solvent is tetrahydrofuran. In some embodiments, the first solvent is methyl ethyl ketone. In other embodiments, the first solvent is water. In some embodiments, the first solvent is a combination of acetone and water. In some embodiments, the first solvent is a combination of methanol and water. In some embodiments, the first solvent combination comprises a volume to volume (v/v) solvent ratio selected from 99/1, 95/5, 9/1, 85/15, 8/2, 7/3, 6/4, 1/1, 4/6, 3/7, 2/8, 15/85, 1/9, 5/95, or 1/99, or a range between any two of these values. In some embodiments, the first solvent is a combination of acetone/water (95:5 v/v). In some embodiments, the first mixture may be stirred at 20, 25, 30, 35, 40, 45, or 50° C., or a range between any two of these values. In some embodiments, the first mixture may be stirred for 1, 2, 3, 4, 5, 6, or 7 days or more. In some embodiments, a second solvent may be added to the first mixture to form a suspension. In some embodiments, the second solvent is selected from ethanol, ethyl acetate, acetonitrile, tetrahydrofuran, methyl ethyl ketone, and water. In some embodiments, the second solvent is a combination of acetone and water. In some embodiments, the second solvent is a combination of methanol and water. In some embodiments, the second solvent combination comprises a solvent ratio that is similar to the solvent ratio of the first solvent combination. In some embodiments, the second solvent combination comprises a solvent ratio that is the same as the solvent ratio of the first solvent combination. In some embodiments, the second solvent may be added to the first mixture over a period of 5, 10, 15, 20, 25, or 30 minute or more. In some embodiments, a slurry is formed upon addition of the second solvent may be added to the first mixture. The method described herein can be used to prepare crystalline Form 1, crystalline Form 2, or a combination of the foregoing. In some embodiments, the method is used to prepare crystalline Form 1. In some embodiments, the method is used to prepare crystalline Form 2.

In some embodiments, a crystalline form of Compound 1-B may be prepared by combining Compound 1 with a solvent and an amount of N-methylglucamine to form a suspension. In some embodiments, the N-methylglucamine can be a single enantiomer. In some embodiments, the N-methylglucamine can be a mixture of stereoisomers. In some embodiments, the amount of N-methylglucamine is sufficient to provide a molar ratio of N-methylglucamine to Compound 1 that is 1:1, 1.05:1, 1.1:1, 1.2:1, 1.3:1, 1.4:1, 1.5:1, 2:1, 3:1, or a range between any two of these values. In some embodiments, the molar ratio of N-methylglucamine to Compound 1 is from 1:1 to 1.1:1. In some specific embodiments, molar ratio of N-methylglucamine to Compound 1 is from 1.0:1.0 to 1.05:1.0. In some specific embodiments, molar ratio of N-methylglucamine to Compound 1 is 1.0:1.0. The suspension may be stirred at room temperature for 1, 2, 3, 4, 5, or more days. In some embodiments, the suspension may be sonicated prior to addition of N-methylglucamine. In some embodiments, the solvent is acetone. In other embodiments, the solvent is acetonitrile. In other embodiments, the solvent is 1,4-dioxane. In yet other embodiments, the solvent is diethyl ether. In some embodiments, the solvent is isopropanol. In other embodiments, the solvent is tetrahydrofuran. In some embodiments, the solvent is methyl ethyl ketone. In some embodiments, the solvent is ethanol. In other embodiments, the solvent is water. In some embodiments, the solvent is a combination of acetone and water. In some embodiments, the solvent is a combination of methanol and water. In some embodiments, the solvent combination comprises a volume to volume (v/v) solvent ratio selected from 99/1, 95/5, 9/1, 85/15, 8/2, 7/3, 6/4, 1/1, 4/6, 3/7, 2/8, 15/85, 1/9, 5/95, or 1/99, or a range between any two of these values. In some embodiments, the solvent is a combination of acetone/water (95:5 v/v). The method described herein can be used to prepare crystalline Form 3, crystalline Form 4, or a combination of the foregoing. In some embodiments, the method is used to prepare crystalline Form 3. In some embodiments, the method is used to prepare crystalline Form 4.

In some embodiments, a crystalline form of Compound 1-B may be prepared by combining Compound 1 with a first solvent and an amount of N-methylglucamine to form a first mixture. In some embodiments, the N-methylglucamine can be a single enantiomer. In some embodiments, the N-methylglucamine can be a mixture of stereoisomers. In some embodiments, the amount of N-methylglucamine is sufficient to provide a molar ratio of N-methylglucamine to Compound 1 that is 1:1, 1.05:1, 1.1:1, 1.2:1, 1.3:1, 1.4:1, or 1:5 to 1. In some embodiments, the molar ratio of N-methylglucamine to Compound 1 is from 1:1 to 1.1:1. In some specific embodiments, molar ratio of N-methylglucamine to Compound 1 is from 1.0:1.0 to 1.05:1.0. In some specific embodiments, molar ratio of N-methylglucamine to Compound 1 is 1.0:1.0. In some embodiments, the first solvent is ethanol. In some embodiments, the first solvent is acetone. In some embodiments, the first solvent is ethyl acetate. In some embodiments, the first solvent is acetonitrile. In other embodiments, the first solvent is tetrahydrofuran. In some embodiments, the first solvent is methyl ethyl ketone. In other embodiments, the first solvent is water. In some embodiments, the first solvent is a combination of acetone and water. In some embodiments, the first solvent is a combination of methanol and water. In some embodiments, the first solvent combination comprises a volume to volume (v/v) solvent ratio selected from 99/1, 95/5, 9/1, 85/15, 8/2, 7/3, 6/4, 1/1, 4/6, 3/7, 2/8, 15/85, 1/9, 5/95, or 1/99, or a range between any two of these values. In some embodiments, the first solvent is a combination of acetone/water (95:5 v/v). In some embodiments, the first mixture may be stirred at 20, 25, 30, 35, 40, 45, or 50° C., or a range between any two of these values. In some embodiments, the first mixture may be stirred for 1, 2, 3, 4, 5, 6, or 7 days or more. In some embodiments, a second solvent may be added to the first mixture to form a suspension. In some embodiments, the second solvent is selected from ethanol, ethyl acetate, acetonitrile, tetrahydrofuran, methyl ethyl ketone, and water. In some embodiments, the second solvent is a combination of acetone and water. In some embodiments, the second solvent is a combination of methanol and water. In some embodiments, the second solvent combination comprises a solvent ratio that is similar to the solvent ratio of the first solvent combination. In some embodiments, the second solvent combination comprises a solvent ratio that is the same as the solvent ratio of the first solvent combination. In some embodiments, the second solvent may be added to the first mixture over a period of 5, 10, 15, 20, 25, or 30 minute or more. In some embodiments, a slurry is formed upon addition of the second solvent may be added to the first mixture. The method described herein can be used to prepare crystalline Form 3, crystalline Form 4, or a combination of the foregoing. In some embodiments, the method is used to prepare crystalline Form 3. In some embodiments, the method is used to prepare crystalline Form 4.

### Definitions

“Subject” as used herein, means a human or a non-human mammal including but not limited to a dog, cat, horse, donkey, mule, cow, domestic buffalo, camel, llama, alpaca, bison, yak, goat, sheep, pig, elk, deer, domestic antelope, or a non-human primate selected for treatment or therapy.

“Subject in need thereof” means a subject identified as in need of a therapy or treatment.

“Treat,” “treatment,” or “treating,” as used herein refers to administering a pharmaceutical composition for prophylactic and/or therapeutic purposes. The term “prophylactic treatment” refers to treating a patient who does not yet have the relevant disease or disorder, but who is susceptible to, or otherwise at risk of, a particular disease or disorder, whereby the treatment reduces the likelihood that the patient will develop the disease or disorder. The term “therapeutic treatment” refers to administering treatment to a patient already having a disease or disorder.

“Preventing” or “prevention” refers to delaying or forestalling the onset, development or progression of a condition or disease for a period of time, including weeks, months, or years.

“Amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“Administering” means providing a pharmaceutical agent or composition to a subject, and includes, but is not limited to, administering by a medical professional and self-administering.

The term “agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances.

“Pharmaceutical agent” means a substance that provides a therapeutic effect when administered to a subject.

“Pharmaceutical composition” means a mixture of substances suitable for administering to an individual that includes a pharmaceutical agent.

“Solvate” refers to the compound formed by the interaction of a solvent and an API, a metabolite, or salt thereof. Suitable solvates are pharmaceutically acceptable solvates including hydrates.

### Pharmaceutical Compositions

Compound 1, Compound 1-A, Compound 1-B, and crystal forms thereof described herein can be formulated into pharmaceutical compositions for use in treatment of the conditions described herein. Standard pharmaceutical formulation techniques are used, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of a compound described herein, or pharmaceutically acceptable salts thereof; and (b) a pharmaceutically acceptable carrier, diluent, excipient or combination thereof.

In some embodiments, the crystal forms of Compound 1, Compound 1-A, and Compound 1-B described herein may be formulated into a single pharmaceutical composition for use in treatment of the conditions described herein. In some embodiments, a formulation comprising Compound 1, Compound 1-A, and/or Compound 1-B, and crystal forms thereof described herein may be administered in combination with one or more second pharmaceutical agents.

In some embodiments, Compound 1, Compound 1-A, and/or Compound 1-B, and crystal forms thereof described herein may be dissolved in a solvent prior to administration to a subject in need thereof.

The term “pharmaceutically acceptable carrier” or “pharmaceutically acceptable excipient” includes any and all solvents, diluents, emulsifiers, binders, buffers, dispersion media, coatings, antibacterial and antifungal agents, isotonic and absorption delaying agents and the like, or any other such compound as is known by those of skill in the art to be useful in preparing pharmaceutical formulations. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, NJ. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such as sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is determined by the way the compound is to be administered.

The compositions described herein are preferably provided in unit dosage form. As used herein, a “unit dosage form” is a composition containing an amount of a compound that is suitable for administration to a subject, in a single dose, according to good medical practice. The preparation of a single or unit dosage form however, does not imply that the dosage form is administered once per day or once per course of therapy. A unit dosage form may comprise a single daily dose or a fractional sub-dose wherein several unit dosage forms are to be administered over the course of a day in order to complete a daily dose. According to the present disclosure, a unit dosage form may be given more or less often that once daily, and may be administered more than once during a course of therapy. Such dosage forms may be administered in any manner consistent with their formulation, including orally, parenterally, and may be administered as an infusion over a period of time (e.g., from about 30 minutes to about 2-6 hours). While single administrations are specifically contemplated, the compositions administered according to the methods described herein may also be administered as a continuous infusion or via an implantable infusion pump.

The methods as described herein may utilize any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropes, surface-active agents, and encapsulating substances. Optional pharmaceutically-active materials may be included, which do not substantially interfere with the activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid, microcrystalline cellulose, carboxymethyl cellulose, and talc. Tablets may also comprise solubilizers or emulsifiers, such as poloxamers, cremophor/Kolliphor®/Lutrol®, methylcellulose, hydroxypropylmethylcellulose, or others as are known in the art. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which can be readily made by a person skilled in the art.

Peroral (PO) compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, onc or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort may be maximized as much as possible, although sometimes formulation considerations (e.g. drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Ophthalmically acceptable antioxidants include, but are not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, including for transdermal administration, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, Compound 1, Compound 1-A, and/or Compound 1-B, and crystal forms thereof described herein and compositions thereof described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J. Pharm. Sci. Tech. 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual unit dose of Compound 1, Compound 1-A, and/or Compound 1-B, and crystal forms thereof described herein depends on the specific compound, and on the condition to be treated. In some embodiments, the dose may be from about 0.01 mg/kg to about 120 mg/kg or more of body weight, from about 0.05 mg/kg or less to about 70 mg/kg, from about 0.1 mg/kg to about 50 mg/kg of body weight, from about 1.0 mg/kg to about 10 mg/kg of body weight, from about 5.0 mg/kg to about 10 mg/kg of body weight, or from about 10.0 mg/kg to about 20.0 mg/kg of body weight. In some embodiments, the dose may be less than 100 mg/kg, 90 mg/kg, 80 mg/kg, 70 mg/kg, 60 mg/kg, 50 mg/kg, 40 mg/kg, 30 mg/kg, 25 mg/kg, 20 mg/kg, 10 mg/kg, 7.5 mg/kg, 6 mg/kg, 5 mg/kg, 4 mg/kg, 3 mg/kg, 2.5 mg/kg, 1 mg/kg, 0.5 mg/kg, 0.1 mg/kg, 0.05 mg/kg or 0.005 mg/kg of body weight. In some embodiments, the actual unit dose is 0.05, 0.07, 0.1, 0.3, 1.0, 3.0, 5.0, 10.0 or 25.0 mg/kg of body weight, or a range between any two of these values. Thus, for administration to a 70 kg person, the dosage range would be from about 0.1 mg to 70 mg, from about 1 mg to about 50 mg, from about 0.5 mg to about 10 mg, from about 1 mg to about 10 mg, from about 2.5 mg to about 30 mg, from about 35 mg or less to about 700 mg or more, from about 7 mg to about 600 mg, from about 10 mg to about 500 mg, or from about 20 mg to about 300 mg, or from about 200 mg to about 2000 mg. In some embodiments, the actual unit dose is 0.1 mg. In some embodiments, the actual unit dose is 0.5 mg. In some embodiments, the actual unit dose is 1 mg. In some embodiments, the actual unit dose is 1.5 mg. In some embodiments, the actual unit dose is 2 mg. In some embodiments, the actual unit dose is 2.5 mg. In some embodiments, the actual unit dose is 3 mg. In some embodiments, the actual unit dose is 3.5 mg. In some embodiments, the actual unit dose is 4 mg. In some embodiments, the actual unit dose is 4.5 mg. In some embodiments, the actual unit dose is 5 mg. In some embodiments the actual unit dose is 10 mg. In some embodiments, the actual unit dose is 25 mg. In some embodiments, the actual unit dose is 250 mg or less. In some embodiments, the actual unit dose is 100 mg or less. In some embodiments, the actual unit dose is 70 mg or less.

In some embodiments, Compound 1, Compound 1-A, and/or Compound 1-B, and crystal forms thereof described herein is administered at a dose in the range of about 1-50 mg/m2 of the body surface area. In some embodiments, Compound 1, Compound 1-A, and/or Compound 1-B, and crystal forms thereof described herein may be administered at a dose in the range of about 1-2, 1-3, 1-4, 1-5, 1-6, 1-7, 1-8, 1-9, 1-10, 1-11, 1-12, 1-13, 1-13.75, 1-14, 1-15, 1-16, 1-17, 1-18, 1-19, 1-20, 1-22.5, 1-25, 1-27.5, 1-30, 1.5-2, 1.5-3, 1.5-4, 1.5-5, 1.5-6, 1.5-7, 1.5-8, 1.5-9, 1.5-10, 1.5-11, 1.5-12, 1.5-13, 1.5-13.75, 1.5-14, 1.5-15, 1.5-16, 1.5-17, 1.5-18, 1.5-19, 1.5-20, 1.5-22.5, 1.5-25, 1.5-27.5, 1.5-30, 2.5-2, 2.5-3, 2.5-4, 2.5-5, 2.5-6, 2.5-7, 2.5-8, 2.5-9, 2.5-10, 2.5-11, 2.5-12, 2.5-13, 2.5-13.75, 2.5-14, 2.5-15, 2.5-16, 2.5-17, 2.5-18, 2.5-19, 2.5-20, 2.5-22.5, 2.5-25, 2.5-27.5, 2.5-30, 2.5-7.5, 3-4, 3-5, 3-6, 3-7, 3-8, 3-9, 3-10, 3-11, 3-12, 3-13, 3-13.75, 3-14, 3-15, 3-16, 3-17, 3-18, 3-19, 3-20, 3-22.5, 3-25, 3-27.5, 3-30, 3.5-6.5, 3.5-13.75, 3.5-15, 2.5-17.5, 4-5, 4-6, 4-7, 4-8, 4-9, 4-10, 4-11, 4-12, 4-13, 4-13.75, 4-14, 4-15, 4-16, 4-17, 4-18, 4-19, 4-20, 4-22.5, 4-25, 4-27.5, 4-30, 5-6, 5-7, 5-8, 5-9, 5-10, 5-11, 5-12, 5-13, 5-13.75, 5-14, 5-15, 5-16, 5-17, 5-18, 5-19, 5-20, 5-22.5, 5-25, 5-27.5, 5-30, 6-7, 6-8, 6-9, 6-10, 6-11, 6-12, 6-13, 6-13.75, 6-14, 6-15, 6-16, 6-17, 6-18, 6-19, 6-20, 6-22.5, 6-25, 6-27.5, 6-30, 7-8, 7-9, 7-10, 7-11, 7-12, 7-13, 7-13.75, 7-14, 7-15, 7-16, 7-17, 7-18, 7-19, 7-20, 7-22.5, 7-25, 7-27.5, 7-30, 7.5-12.5, 7.5-13.5, 7.5-15, 8-9, 8-10, 8-11, 8-12, 8-13, 8-13.75, 8-14, 8-15, 8-16, 8-17, 8-18, 8-19, 8-20, 8-22.5, 8-25, 8-27.5, 8-30, 9-10, 9-11, 9-12, 9-13, 9-13.75, 9-14, 9-15, 9-16, 9-17, 9-18, 9-19, 9-20, 9-22.5, 9-25, 9-27.5, 9-30, 10-11, 10-12, 10-13, 10-13.75, 10-14, 10-15, 10-16, 10-17, 10-18, 10-19, 10-20, 10-22.5, 10-25, 10-27.5, 10-30, 11.5-15.5, 12.5-14.5, 7.5-22.5, 8.5-32.5, 9.5-15.5, 15.5-24.5, 5-35, 17.5-22.5, 22.5-32.5, 25-35, 25.5-24.5, 27.5-32.5, 2-20, 2.5-22.5, or 9.5-21.5 mg/m2, of the body surface area. In some embodiments, Compound 1, Compound 1-A, and/or Compound 1-B, and crystal forms thereof described herein may be administered at a dose of about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, Compound 1, Compound 1-A, and/or Compound 1-B, and crystal forms thereof described herein may be administered at a dose less than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, Compound 1, Compound 1-A, and/or Compound 1-B, and crystal forms thereof described herein may be administered at a dose greater than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50 mg/m² of the body surface area.

In some embodiments, the dose of Compound 1, Compound 1-A, and/or Compound 1-B, and crystal forms thereof described herein may be about 0.1 mg-100 mg, 0.1 mg-50 mg, 0.1 mg-20 mg, 0.1 mg-10 mg, 0.5 mg-100 mg, 0.5 mg-50 mg, 0.5 mg-20 mg, 0.5 mg-10 mg, 1 mg-100 mg, 1 mg-50 mg, 1 mg-20 mg, 1 mg-10 mg, 2.5 mg-50 mg, 2.5 mg-20 mg, 2.5 mg-10 mg, or about 2.5 mg-5 mg. In some embodiments, the dose of Compound 1, Compound 1-A, and/or Compound 1-B, and crystal forms thereof described herein may be about 5 mg-300 mg, 5 mg-200 mg, 7.5 mg-200 mg, 10 mg-100 mg, 15 mg 100 mg, 20 mg-100 mg, 30 mg-100 mg, 40 mg-100 mg, 10 mg-80 mg, 15 mg-80 mg, 20 mg-80 mg, 30 mg-80 mg, 40 mg-80 mg, 10 mg-60 mg, 15 mg-60 mg, 20 mg-60 mg, 30 mg-60 mg, or about 40 mg-60 mg. In some embodiments, the dose of Compound 1, Compound 1-A, and/or Compound 1-B, and crystal forms thereof described herein may be about 20 mg-60 mg, 27 mg-60 mg, 20 mg-45 mg, or 27 mg-45 mg. In some embodiments, the dose of the crystal forms of Compound 1, Compound 1-A, and Compound 1-B described herein may be about 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, the dose of Compound 1, Compound 1-A, and/or Compound 1-B, and crystal forms thereof described herein may be greater than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, the dose of Compound 1, Compound 1-A, and/or Compound 1-B, and crystal forms thereof described herein may be less than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, the dose of Compound 1, Compound 1-A, and/or Compound 1-B, and crystal forms thereof described herein may be about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, about 200 mg, about 225 mg, about 250 mg, about 275 mg, or about 300 mg, or a range between any two of these values.

Compound 1, Compound 1-A, and/or Compound 1-B, and crystal forms thereof described herein may also be incorporated into formulations for delivery outside the systemic circulation. Such formulations may include enteric-coated capsules, tablets, soft-gels, spray dried powders, polymer matrices, hydrogels, enteric-coated solids, crystalline solids, amorphous solids, glassy solids, coated micronized particles, liquids, nebulized liquids, aerosols, or microcapsules.

### Methods of Administration

The compositions described above may be administered through any suitable route of administration, for example, by injection, such as subcutaneously, intramuscularly, intraperitoneally, intravenously, or intraarterially; topically, such as by cream, lotion, or patch; orally, such as by a pill, dissolved liquid, oral suspension, buccal film, or mouth rinse; nasally, such as by a nasal aerosol, powder, or spray; or ocularly, such as by an eye drop). In some embodiments, the composition may be administered one, twice, three times, our four times per day. In other embodiments, the composition may be administered once, twice, or three times per week. In other embodiments, the composition is administered every other day, every three days, or every four days. In other embodiments, the composition every other week, every three weeks, or every four weeks. In other embodiments, the composition is administered once per month or twice per month.

In some embodiments, Compound 1, Compound 1-A, and/or Compound 1-B, and crystal forms thereof described herein may be administered simultaneously with one or more second pharmaceutical agents. In other embodiments, Compound 1, Compound 1-A, and/or Compound 1-B, and crystal forms thereof described herein may be administered sequentially with one or more second pharmaceutical agents.

### Methods of Treatment

Some embodiments relate to a method for preventing, treating, or ameliorating one or more disease or condition associated with autotaxin activity by administering a compound or composition described herein.

### Fibrosis

In some embodiments, disclosed herein are methods of treating fibrosis with a compound or composition described herein. In some embodiments, disclosed herein are methods of preventing fibrosis with a compound or composition described herein. In some embodiments, disclosed herein are methods of attenuating, reversing, or inhibiting a sign or symptom of fibrosis.

In some embodiments, disclosed herein are methods comprising the administration of a compound or composition described herein to a subject having fibrosis. In some embodiments, disclosed herein are methods comprising the administration of a compound or composition described herein to a subject susceptible to fibrosis.

In some embodiments, disclosed herein are methods of reducing fibrosis in a tissue comprising contacting a fibrotic cell or tissue with a compound or composition described herein, in an amount sufficient to decrease and/or inhibit the fibrosis. In some embodiments, the fibrosis includes a fibrotic condition. In some cases, the amount sufficient to decrease and/or inhibit fibrosis is a therapeutically effective amount.

In some embodiments, reducing fibrosis, or treatment of a fibrotic condition, includes reducing or inhibiting one or more of: formation or deposition of extracellular matrix proteins; the number of pro-fibrotic cell types (e.g., fibroblast or immune cell numbers); cellular collagen or hydroxyproline content within a fibrotic lesion; expression or activity of a fibrogenic protein; reducing fibrosis associated with an inflammatory response; or a combination thereof.

“Fibrosis,” as used herein, refers to the accumulation of extracellular matrix constituents that occurs following trauma, inflammation, tissue repair, immunological reactions, cellular hyperplasia, and/or neoplasia. Examples of tissue fibrosis include, but are not limited to, pulmonary fibrosis, renal fibrosis, cardiac fibrosis, cirrhosis and fibrosis of the liver, ocular fibrosis, skin scars and keloids, kidney fibrosis, peritoneal fibrosis, adhesions, fibromatosis, atherosclerosis, and amyloidosis.

In some embodiments, the fibrotic condition is primary fibrosis. In some embodiments, the fibrotic condition is idiopathic. In some embodiments, the fibrotic condition is associated with (e.g., is secondary to) a disease (e.g., an infectious disease, an inflammatory disease, an autoimmune disease, a malignant or cancerous disease, and/or a connective disease); a toxin; an insult (e.g., an environmental hazard (e.g., asbestos, coal dust, polycyclic aromatic hydrocarbons), cigarette smoking, a wound); a medical treatment (e.g., surgical incision, chemotherapy or radiation), or a combination thereof.

In some embodiments, the fibrotic condition is a fibrotic condition of the lung, a fibrotic condition of the liver, a fibrotic condition of the heart or vasculature, a fibrotic condition of the kidney, a fibrotic condition of the skin, a fibrotic condition of the gastrointestinal tract, a fibrotic condition of the eye, a fibrotic condition of the bone marrow or a hematopoietic tissue, a fibrotic condition of the nervous system, fibrotic condition of the peritoneum, or a combination thereof.

In some embodiments, the fibrotic condition affects a tissue chosen from one or more of muscle, tendon, cartilage, skin (e.g., skin epidermis or endodermis), cardiac tissue, vascular tissue (e.g., artery, vein), pancreatic tissue, lung tissue, liver tissue, kidney tissue, uterine tissue, ovarian tissue, neural tissue, testicular tissue, peritoneal tissue, colon, small intestine, biliary tract, gut, bone marrow, or hematopoietic tissue.

In some embodiments, the fibrotic condition is a fibrotic condition of the liver. In certain embodiments, the fibrotic condition of the liver is chosen from one or more of: fatty liver disease, steatosis (e.g., nonalcoholic steatohepatitis (NASH)), cholestatic liver disease (e.g., primary biliary cirrhosis (PBC)), cirrhosis, alcohol-induced liver fibrosis, biliary duct injury, biliary fibrosis, cholestasis or cholangiopathies. In some embodiments, hepatic or liver fibrosis includes, but is not limited to, hepatic fibrosis associated with alcoholism, viral infection, e.g., hepatitis (e.g., hepatitis C, B or D), autoimmune hepatitis, non-alcoholic fatty liver disease (NAFLD), progressive massive fibrosis, and exposure to toxins or irritants (e.g., alcohol, pharmaceutical drugs and environmental toxins).

In some embodiments, described herein are methods for the treatment or prevention of fibrosis of the liver in a subject, the methods comprising administration of a compound or composition described herein to the subject. In some instances, the fibrosis is chronic or acute. In some cases, administration of a compound or composition described herein results in the attenuation, delay or prevention of a sign, symptom and/or complication of liver fibrosis. For example, the method attenuates, delays or prevents the onset of cirrhosis, liver failure, portal hypertension, and/or complications thereof. Exemplary complications of cirrhosis include ascites, renal failure, hepatic encephalopathy, and variceal bleeding. In some cases, administration of a compound or composition described herein inhibits the accumulation of fibrogenic cells and/or prevents the deposition of extracellular matrix proteins (e.g., collagen) within the liver. Autotaxin inhibitors used in a method provided herein are useful for the treatment of liver fibrosis caused by any source of liver damage. Examples of liver fibrosis causes include, without limitation, viral infection (e.g., hepatitis C), autoimmune hepatitis, congenital hepatic fibrosis, bacterial infection (e.g., Brucellosis), parasitic infection (e.g., Echinococcosis), alcohol abuse, primary sclerosing cholangitis, drugs (e.g., amiodarone, chlorpromaxine, isoniazid, methotrexate, methyldopa, oxyphenisatin, tolbutamide), mechanical obstruction (e.g., surgery) nonalcoholic steatohepatitis (NASH), and combinations thereof.

In some cases, described herein are methods for the reversal of fibrosis or fibrosis regression of the liver in a subject, the methods comprising administration of a compound or composition described herein to the subject.

In some embodiments, any method provided herein comprising the administration of a compound or composition described herein, further comprises the administration of one or more additional fibrosis therapies. Additional fibrosis therapies include, for example, the administration of an angiotensin inhibitor, colchicine, corticosteroid, endothelin inhibitor, interferon-alpha, interleukin 10, antioxidant, hepatic stellate cell (HSC) inhibitor, or combination thereof.

In some embodiments, the fibrotic condition is a fibrotic condition of the kidney. In some embodiments, the fibrotic condition of the kidney is chosen from one or more of: renal fibrosis (e.g., chronic kidney fibrosis), nephropathies associated with injury/fibrosis (e.g., chronic nephropathies associated with diabetes (e.g., diabetic nephropathy)), lupus, scleroderma of the kidney, glomerular nephritis, focal segmental glomerular sclerosis, IgA nephropathy renal fibrosis associated with human chronic kidney disease (CKD), chronic progressive nephropathy (CPN), tubulointerstitial fibrosis, ureteral obstruction, chronic uremia, chronic interstitial nephritis, radiation nephropathy, glomerulosclerosis, progressive glomerulonephrosis (PGN), endothelial/thrombotic microangiopathy injury, HIV-associated nephropathy, or fibrosis associated with exposure to a toxin, an irritant, or a chemotherapeutic agent.

In some embodiments, described herein are methods for the treatment or prevention of fibrosis of the kidney in a subject, the methods comprising the administration of a compound or composition described herein to the subject. In some instances, administration of a compound or composition described herein to the subject attenuates, delays or inhibits the progression of kidney fibrosis. In some cases, administration of a compound or composition described herein results in the attenuation, delay or prevention of a sign, symptom and/or complication of kidney fibrosis. In some instances, administration of a compound or composition described herein reduces the accumulation of extracellular matrix or the rate of accumulation of extracellular matrix in the kidney. In some cases, kidney fibrosis is the result of acute or chronic, sustained injury to kidney tissue. In some cases, kidney fibrosis is characterized by glomerulosclerosis. In some cases, kidney fibrosis is characterized by tubulointerstitial fibrosis.

In some cases, described herein are methods for the reversal of fibrosis or fibrosis regression of the kidney in a subject, the methods comprising administration of a compound or composition described herein to the subject.

In some embodiments, any method provided herein comprising the administration of a compound or composition described herein, further comprises the administration of one or more additional fibrosis and/or kidney disease therapies. An additional therapy comprises, for example, inhibiting profibrotic TGF-β/Smad signaling. As another example, an additional therapy comprises increasing the concentration of antifibrotic factors in the kidney, for example, hepatocyte growth factor (HGF) and bone morphogenetic protein-7 (BMP-7), which can antagonize fibrogenic action of TGF-β. Exemplary fibrosis treatments also include, without limitation, the administration of one or more of: pirfenidone (5-methyl-N-phenyl-2-(1H)-pyridone), tranilast, fluorofenidone, blockade of renin-angiotensin-aldosterone, ACE inhibitors, ADAM inhibitors, and an anti-CTGF monoclonal antibody.

In some embodiments, the fibrotic condition is a fibrotic condition of the skin. In some embodiments, the fibrotic condition of the skin is chosen from one or more of: skin fibrosis, scleroderma, nephrogenic systemic fibrosis (e.g., resulting after exposure to gadolinium which is frequently used as a contrast substance for MRIs in patients with severe kidney failure), scarring and keloid.

In some embodiments, described herein are methods for the treatment or prevention of fibrosis of the skin in a subject, the methods comprising the administration of a compound or composition described herein to the subject. In some instances, administration of a compound or composition described herein to the subject attenuates, delays or inhibits the progression of skin fibrosis. In some cases, administration of a compound or composition described herein results in the attenuation, delay or prevention of a sign, symptom and/or complication of skin fibrosis. In some instances, administration of a compound or composition described herein reduces the accumulation of extracellular matrix or the rate of accumulation of extracellular matrix in the skin. In some cases, skin fibrosis is the result of acute or chronic, sustained injury to skin tissue.

In some embodiments, following administration of a compound or composition described herein to a subject having skin fibrosis, the dermal thickness of the fibrotic skin decreases. For example, administration of a compound or composition described herein decreases dermal thickness of skin fibrosis by at least about 5%, 10%, 20%, 30%, 40% or 50% as compared to dermal thickness prior to autotaxin inhibitor administration.

In some embodiments, described herein are methods for the reversal of fibrosis or fibrosis regression of the skin of a subject, the methods comprising administration of a compound or composition described herein to the subject.

In some embodiments, any method provided herein comprising the administration of a compound or composition described herein, further comprises the administration of one or more additional fibrosis and/or skin disease therapies. Skin fibrosis therapies include, without limitation, administration of D-penicillamine, bovine collagen, methotrexate, mycophenolate mofetil, human relaxin, interferon-alpha, anti-transforming growth factor beta antibodies, or a combination thereof.

In some embodiments, the fibrotic condition is a fibrotic condition of the eye. In some embodiments, the fibrotic condition of the eye is chosen from one or more of: glaucoma, subretinal fibrosis, age-related macular degeneration (ARMD), diabetic retinopathy (DR), retinopathy of prematurity (ROP), fibrosis following eye surgery (glaucoma surgery, cataract surgery, LASIK surgery).

In some embodiments, described herein are methods for the treatment or prevention of fibrosis of the eye of a subject, the methods comprising the administration of a compound or composition described herein to the subject. In some instances, administration of a compound or composition described herein to the subject attenuates, delays or inhibits the progression of ocular fibrosis. In some cases, administration of a compound or composition described herein results in the attenuation, delay or prevention of a sign, symptom and/or complication of ocular fibrosis. In some instances, administration of a compound or composition described herein reduces the accumulation of extracellular matrix or the rate of accumulation of extracellular matrix in the eye. In some cases, ocular fibrosis is the result of acute or chronic, sustained injury to eye tissue.

In some embodiments, following administration of a compound or composition described herein to a subject having ocular fibrosis, the extent of fibrosis of the eye decreases. For example, administration of a compound or composition described herein decreases fibrosis of the bleb following trabeculectomy surgery and reduces the failure rate of the bleb due to fibrosis by about 5%, 10%, 20%, 30%, 40% or 50% as compared to patients without treatment with a compound or composition described herein.

In some embodiments, described herein are methods for the reversal of fibrosis or fibrosis regression of the eye of a subject, the methods comprising administration of a compound or composition described herein to the subject.

In some embodiments, any method provided herein comprising the administration of a compound or composition described herein, further comprises the administration of one or more additional fibrosis and/or eye disease therapies. Eye fibrosis therapies include, without limitation, administration of mitomycin, 5-fluorouracil, corticosteroids, antibiotics, anti-transforming growth factor beta antibodies, or a combination thereof.

In some embodiments, the fibrotic condition is a fibrotic condition of the peritoneum. In some embodiments, described herein are methods for the treatment or prevention of fibrosis of the peritoneum a subject, the methods comprising the administration of a compound or composition described herein to the subject. In some instances, administration of a compound or composition described herein to the subject attenuates, delays or inhibits the progression of peritoneal fibrosis. In some cases, administration of a compound or composition described herein results in the attenuation, delay or prevention of a sign, symptom and/or complication of peritoneal fibrosis. In some instances, administration of a compound or composition described herein reduces the accumulation of extracellular matrix or the rate of accumulation of extracellular matrix in the peritoneum.

In some embodiments, fibrosis of the peritoneum is caused by long-term peritoneal dialysis. In some embodiments, a compound or composition described herein is administered to a subject having peritonitis. In some embodiments, peritoneal fibrosis is caused by one or more of the following insults: bioincompatible dialysates, peritonitis, uremia, and/or chronic inflammation.

In some cases, described herein are methods for the reversal of fibrosis or fibrosis regression of the peritoneum of a subject, the methods comprising administration of a compound or composition described herein to the subject.

In some embodiments, any method provided herein comprising the administration of a compound or composition described herein, further comprises the administration of an anti-inflammatory agent or an immunosuppressant.

In some embodiments, the fibrotic condition is a fibrotic condition of the gastrointestinal tract. In some embodiments, the fibrotic condition is chosen from one or more of fibrosis associated with scleroderma; radiation induced gut fibrosis; fibrosis associated with a foregut inflammatory disorder such as Barrett's esophagus and chronic gastritis, and/or fibrosis associated with a hindgut inflammatory disorder, such as inflammatory bowel disease (IBD), ulcerative colitis and Crohn's disease.

In some embodiments, the fibrotic condition is a fibrotic condition of the lung. In some embodiments, the fibrotic condition of the lung is chosen from one or more of: pulmonary fibrosis, idiopathic pulmonary fibrosis (IPF), usual interstitial pneumonitis (UIP), interstitial lung disease, cryptogenic fibrosing alveolitis (CFA), bronchiolitis obliterans, or bronchiectasis. In some embodiments, the fibrosis of the lung is secondary to a disease, a toxin, an insult, a medical treatment, or a combination thereof. In some embodiments, fibrosis of the lung is associated with one or more of: a disease process such as asbestosis and silicosis; an occupational hazard; an environmental pollutant; cigarette smoking; an autoimmune connective tissue disorders (e.g., rheumatoid arthritis, scleroderma and systemic lupus erythematosus (SLE)); a connective tissue disorder such as sarcoidosis; an infectious disease, e.g., infection, particularly chronic infection; a medical treatment, including but not limited to, radiation therapy, and drug therapy, e.g., chemotherapy (e.g., treatment with as bleomycin, methotrexate, amiodarone, busulfan, and/or nitrofurantoin). In some embodiments, the fibrotic condition of the lung treated with the methods of the invention is associated with (e.g., secondary to) a cancer treatment, e.g., treatment of a cancer (e.g. squamous cell carcinoma, testicular cancer, Hodgkin's disease with bleomycin).

In some embodiments, the fibrotic condition is a fibrotic condition of the heart. In certain embodiments, the fibrotic condition of the heart is myocardial fibrosis (e.g., myocardial fibrosis associated with radiation myocarditis, a surgical procedure complication (e.g., myocardial post-operative fibrosis), infectious diseases (e.g., Chagas disease, bacterial, trichinosis or fungal myocarditis)); granulomatous, metabolic storage disorders (e.g., cardiomyopathy, hemochromatosis); developmental disorders (e.g., endocardial fibroelastosis); arteriosclerotic, or exposure to toxins or irritants (e.g., drug induced cardiomyopathy, drug induced cardiotoxicity, alcoholic cardiomyopathy, cobalt poisoning or exposure). In some embodiments, the myocardial fibrosis is associated with an inflammatory disorder of cardiac tissue (e.g., myocardial sarcoidosis).

In some embodiments, the fibrotic condition is adhesions. In some embodiments, the adhesions are chosen from one or more of: abdominal adhesions, peritoneal adhesions, pelvic adhesions, pericardial adhesions, peridural adhesions, peritendinous or adhesive capsulitis.

In some embodiments, the fibrotic condition is a fibrotic condition of the eye. In some embodiments, the fibrotic condition of the eye involves diseases of the anterior segment of the eye such as glaucoma and corneal opacification; in some embodiments, the fibrotic condition of the eye involves disease of the posterior segment of the eye such as age-related macular degeneration, diabetic retinopathy, retinopathy of prematurity and neovascular glaucoma; in some embodiments, the fibrotic condition of the eye results from fibrosis following ocular surgery.

In some embodiments, the fibrotic condition is a fibrotic condition of the bone marrow or a hematopoietic tissue. In some embodiments, the fibrotic condition of the bone marrow is an intrinsic feature of a chronic myeloproliferative neoplasm of the bone marrow, such as primary myelofibrosis (also referred to herein as agnogenic myeloid metaplasia or chronic idiopathic myelofibrosis). In some embodiments, the bone marrow fibrosis is associated with (e.g., is secondary to) a malignant condition or a condition caused by a clonal proliferative disease. In some embodiments, the bone marrow fibrosis is associated with a hematologic disorder (e.g., a hematologic disorder chosen from one or more of polycythemia vera, essential thrombocythemia, myelodysplasia, hairy cell leukemia, lymphoma (e.g., Hodgkin or non-Hodgkin lymphoma), multiple myeloma or chronic myelogeneous leukemia (CML)). In some embodiments, the bone marrow fibrosis is associated with (e.g., secondary to) a non-hematologic disorder (e.g., a non-hematologic disorder chosen from solid tumor metastasis to bone marrow, an autoimmune disorder (e.g., systemic lupus erythematosus, scleroderma, mixed connective tissue disorder, or polymyositis), an infection (e.g., tuberculosis), or secondary hyperparathyroidism associated with vitamin D deficiency.

### Colitis

In some embodiments, disclosed herein are methods of treating colitis with a compound or composition described herein. In some embodiments, disclosed herein are methods of preventing colitis with a compound or composition described herein. In some embodiments, disclosed herein are methods of attenuating, reversing, or inhibiting a sign or symptom of colitis.

In some embodiments, disclosed herein are methods comprising the administration of a compound or composition described herein to a subject having colitis. In some embodiments, disclosed herein are methods comprising the administration of a compound or composition described herein to a subject susceptible to colitis.

In some embodiments, disclosed herein are methods of reducing colitis in a tissue, the methods comprising contacting a tissue with a compound or composition described herein, in an amount sufficient to decrease or inhibit the colitis. In some cases, the amount sufficient to decrease and/or inhibit colitis is a therapeutically effective amount.

“Colitis,” as used herein, refers to an inflammation of colon tissue. Colitis includes acute, self-limited and chronic colitis. Colitis includes autoimmune colitis, idiopathic colitis, latrogenic colitis, vascular disease and infectious colitis. Autoimmune types of colitis include inflammatory bowel disease and ulcerative colitis. Idiopathic types of colitis include microscopic colitis, lymphocytic colitis and collagenous colitis. Latrogenic types of colitis include diversion colitis and chemical colitis. Colitis caused by vascular disease includes ischemic colitis. Infectious colitis includes colitis caused by Clostridium difficile, Shigella dysenteriae or Shigatoxigenic group of Escherichia coli. In some embodiments, colitis comprises ulcerative colitis.

In some embodiments, disclosed herein are methods of treating colitis in a subject by administering a compound or composition described herein to the subject. In some embodiments, one or more signs or symptoms of colitis are reduced in a subject after administration of a compound or composition described herein. Signs or symptoms of colitis include, without limitation, abdominal pain, loss of appetite, fatigue, diarrhea, mucus in the stool, cramping, urgency, bloating, abdominal tenderness, weight loss, changes in bowel habits such as increased frequency, fever, bleeding, bloody stool, distension, colonic mucosal erythema and ulcers.

In some embodiments, following administration of a compound or composition described herein to a subject having colitis, the subject shows improvement in colitis clinical signs, symptoms or histopathology. In one example, following autotaxin inhibitor administration, colon inflammation is decreased. In another example, following autotaxin inhibitor administration, tissue damage is reversed. In another example, following autotaxin inhibitor administration, tissue damage progression is attenuated. In yet another example, following autotaxin inhibitor administration, tissue damage is inhibited. Tissue damage can be visualized, for example, by histology using a stain such as trichrome stain (Masson).

In some embodiments, following autotaxin inhibitor administration to a subject having ulcerative colitis, ulcer area is decreased, for example, by at least about 1%, 2%, 3%, 4%, 5%, 10%, 15%, 20%, 50%, 90%, 99% of its size prior to autotaxin inhibitor administration. In another example, following autotaxin inhibitor administration, ulcer area is at least about 2-fold, 3-fold or 5-fold smaller in size than prior to autotaxin inhibitor administration or as compared to administration of a control.

In some embodiments, following autotaxin inhibitor administration to a subject having colitis, the subject's body weight does not increase or decrease by more than about 2%, 5% or 10%.

In some embodiments, following autotaxin inhibitor administration to a subject having colitis, the subject has an increase in stool consistency.

In some embodiments, a subject having colitis is treated by administration of a compound or composition described herein and one or more additional treatments. Addition treatments include hydration therapy and administration of a steroid, anti-inflammatory agent, and/or immunosuppressant. In one example, the additional treatment is cyclosporin A (CsA). In another example, the additional treatment is sulfasalazine.

In some embodiments, an animal model induced with colitis (e.g., DSS-induced colitis or DNBS-induced colitis) is administered (prophylactically or therapeutically) a compound or composition described herein; wherein following autotaxin inhibitor administration, the animal exhibits an increase in colon length, an increase in colon weight, or both an increase in colon length and weight. For example, following administration of a compound or composition described herein to a mouse or rat induced with colitis, the colon length increases by at least about 0.1 cm, 0.2 cm, 0.3 cm, 0.4 cm, 0.5 cm, 1 cm, 2 cm or 3 cm as compared to administration of a vehicle or control. In another example, following administration of a compound or composition described herein to a mouse or rat induced with colitis, the colon weight increases by at least about 0.05 g as compared to administration of a vehicle or control. In some embodiments, following autotaxin inhibitor treatment of a mouse or rat induced with colitis, the mouse or rat has a reduction in ulcer area by at least about 10%, 20%, 50% or 90% as compared to ulcer area prior to administration. In some embodiments, following autotaxin inhibitor treatment of a mouse or rat induced with colitis, the mouse or rat has an ulcer area less than about 90%, 80%, 70%, 50%, 20%, or 10% of ulcer area size in an untreated or control mouse or rat having induced colitis.

### Pruritus

In some embodiments, disclosed herein are methods of treating pruritus with a compound or composition described herein. In some embodiments, disclosed herein are methods of preventing pruritus with a compound or composition described herein. In some embodiments, disclosed herein are methods of attenuating, reversing or inhibiting at least one sign or symptom of pruritus.

In some embodiments, disclosed herein are methods comprising the administration of a compound or composition described herein to a subject having pruritus. In some embodiments, disclosed herein are methods comprising the administration of a compound or composition described herein to a subject susceptible to pruritus.

In some embodiments, disclosed herein are methods of reducing pruritus in a tissue, the methods comprising contacting a tissue with a compound or composition described herein, in an amount sufficient to decrease or inhibit the pruritus.

Pruritus is a condition involving localized or general itching that is a common and distressing symptom in a variety of diseases. Although usually occurring in the skin, pruritus can also occur in non-cutaneous sites such as mucous membranes. Pruritus is a frequent manifestation of localized skin disorders caused by hypersensitivity reactions such as allergic reactions to insect bites or to environmental allergens, urticaria, dermatoses of fungal and bacterial origins, ectoparasite infections, and hemorrhoids. In some embodiments, disclosed herein are method of treating pruritus caused by systemic diseases, including, for example, hypothyroidism, thyrotoxicosis, mucocandiasis in diabetes mellitus, and Hodgkin's disease. In some embodiments, disclosed herein are methods of treating bouts of persistent or recurrent pruritus associated with many systemic diseases and skin disorders. Pruritus includes, without limitation, renal pruritus, cholestatic pruritus, hematologic pruritus and endocrine pruritus.

In some embodiments, disclosed herein are methods of treating pruritus associated with liver diseases and intrahepatic or posthepatic cholestasis. Hepatic diseases leading to pruritus include primary biliary cirrhosis, B and C viral hepatitis, primary sclerosing cholangitis, carcinoma of bile ducts, alcoholic cirrhosis, autoimmune hepatitis and others.

In some embodiments, disclosed herein are methods of treating pruritus arising from a variety of causes such as xerosis, skin conditions (such as psoriasis, eczema, sunburn, athlete's foot), insect bites, poisonous plants (such as poison ivy, poison oak, poison sumac), Hodgkin's disease, jaundice, polycythemia, scabies, lice, worms, thyroid illness, diabetes mellitus, dandruff, iron deficiency anemia, parasitic infections, medications, cholestasis, pruritus related to pregnancy, HIV infection, other causes of itching or pruritus, or combinations thereof.

In some embodiments, disclosed herein are methods of treating or preventing pruritus in a subject in need thereof, the methods comprising the administration of a compound or composition described herein to the subject. In some embodiments, administration of a compound or composition described herein reduces pruritus by at least about 10%, 20%, 30%, 50%, or 90%.

In some embodiments, following autotaxin inhibitor administration to a subject having pruritus, the subject has a decrease in total number of scratch movements as compared to scratch number before autotaxin inhibitor administration. For example, scratch movements decrease by at least about 10%, 20%, 50%, or 90%. In some embodiments, pruritus is prevented or treated by the administration of a compound or composition described herein and one or more additional pruritus therapies or therapeutic agents. Pruritus therapies include, without limitation, use of skin creams and lotions to prevent skin dryness and usc of an antihistamine, steroid or antibiotic. Pruritus therapeutic agents include, doxepin, mirtazapine, gabapentin, aprepitant, capsaicin, tacrolimus, gamma linolenic acid, cholestyramine, rifampin, an opioid antagonist, ondansetron, and activated charcoal.

In some embodiments, described herein are methods for the prevention or treatment of renal pruritus in a subject, the methods comprising administration of a compound or composition described herein to the subject. In some embodiments, described herein are methods for the prevention or treatment of cholestatic pruritus in a subject, the methods comprising administration of a compound or composition described herein to the subject. In some embodiments, described herein are methods for the prevention or treatment of hematologic pruritus in a subject, the methods comprising administration of a compound or composition described herein to the subject. In some embodiments, described herein are methods for the prevention or treatment of endocrine pruritus in a subject, the methods comprising administration of a compound or composition described herein to the subject.

### Neurological Disease

In some embodiments, disclosed herein are methods of treating a neurological disease, condition or disorder with a compound or composition described herein. In some embodiments, disclosed herein are methods of preventing a neurological disease, condition or disorder with a compound or composition described herein. In some embodiments, disclosed herein are methods of attenuating, reversing or inhibiting a sign or symptom of a neurological disease, condition or disorder.

In some embodiments, disclosed herein are methods comprising the administration of a compound or composition described herein to a subject having a neurological disease, condition or disorder. In some embodiments, disclosed herein are methods comprising the administration of a compound or composition described herein to a subject susceptible to having a neurological disease, condition or disorder.

In some embodiments, the neurological disease is multiple sclerosis. In some instances, the neurological disease is caused by a genetic disorder. In some instances, the neurological disease is developmental, for example, Spina bifida. In some instances, the neurological disease is a degenerative disease, for example, Parkinson's disease or Alzheimer's disease. In some instances, the neurological disease results from a stroke. Neurological diseases, conditions and disorders or characteristics of neurological diseases, conditions and disorders suitable for treatment with an ATX inhibitor include, without limitation, Amyotrophic lateral sclerosis (ALS), Arteriovenous malformations (AVMs), brain aneurysm, brain tumor, Dural arteriovenous fistulae, epilepsy, headache, memory disorders, Parkinson's disease, peripheral neuropathy, post-herpetic neuralgia, spinal cord tumor and stroke. In certain instances, autotaxin activity is increased in CSF (cerebrospinal fluid) and serum of relapse/remitting multiple sclerosis patients compared to patients with other neurological diseases.

In some embodiments, the multiple sclerosis is relapsing-remitting multiple sclerosis, relapsing multiple sclerosis, primary-progressive multiple sclerosis, or secondary-progressive multiple sclerosis.

In some embodiments, the multiple sclerosis is relapsing-remitting multiple sclerosis (RRMS). People with this type of MS have clearly defined attacks of worsening neurologic function. These attacks are followed by partial or complete recovery periods called remissions. During remission, symptoms often improve and there is no apparent worsening or progression of disease. About 85% of people with MS are initially diagnosed with RRMS.

In some embodiments, the multiple sclerosis is relapsing multiple sclerosis (RMS). RMS includes several forms of MS that have relapsing features, including relapsing-remitting MS, progressive-relapsing MS, and secondary-progressive MS.

In some embodiments, the multiple sclerosis is primary-progressive multiple sclerosis (PPMS). This form has a steady worsening of neurologic functioning, but without any distinct relapses or periods of remission. A person's rate of progression may vary over time—with occasional plateaus or temporary improvements—but the progression is continuous. 10% of people are diagnosed with this type of MS.

In some embodiments, the multiple sclerosis is secondary-progressive multiple sclerosis (SPMS). Following an initial period of relapsing-remitting MS (RRMS), many people transition to SPMS. The disease begins to worsen more steadily, with or without occasional relapses, remissions, or plateaus.

In some embodiments, disclosed herein are methods of treating a nervous system injury in a subject, for example, injury to the brain, spinal cord and/or nerve tissue, the methods comprising the administration of a compound or composition described herein to the subject.

In some embodiments, disclosed herein are methods for the treatment of a cancer affecting the nervous system of a subject, the methods comprising the administration of a compound or composition described herein to the subject. In some examples, the cancer is brain cancer.

In some embodiments, disclosed herein are methods of treating injury-induced demyelination in a subject, the methods comprising the administration of a compound or composition described herein to the subject.

In some embodiments, disclosed herein are methods of treating an infection of the nervous system in a subject, the methods comprising the administration of a compound or composition described herein to the subject. In some examples, the nervous system infection includes meningitis.

In some embodiments, disclosed herein are methods of treating a neurological disease, disorder or condition in a subject, the methods comprising administration of a compound or composition described herein to the subject in need thereof. In some embodiments, disclosed herein are methods of treating multiple sclerosis in a subject, the methods comprising administering a compound or composition described herein to the subject. In some embodiments, treatment includes the inhibition or reversal of demyelination. Inhibition of demyelination includes a decrease in rate of demyelination as compared to the absence of autotaxin inhibitor treatment, where a decrease in rate is a decrease of at least 5%, 10%, 20%, 30%, 40%, 50%, 80% or 90% demyelination. In some instances, administration of a compound or composition described herein prevents, delays and and/or attenuates demyelination.

In some embodiments, disclosed herein are methods of preventing or treating a sign, symptom and/or complication of a neurological disease, disorder or condition, the methods comprising administration of a compound or composition described herein. In some embodiments, disclosed herein are methods of preventing or treating a sign, symptom and/or complication of multiple sclerosis in a subject, the methods comprising administering a compound or composition described herein to the subject. Signs and symptoms of multiple sclerosis include, without limitation, numbness or weakness in one or more limbs, partial or complete loss of vision, double vision, blurring of vision, tingling sensation, electric-shock sensations, tremors, lack of coordination, unsteady gait, slurred speech, fatigue, dizziness, and changes in bowel and/or bladder function. Examples of multiple sclerosis complications include, without limitation, muscle stiffness, muscle spasms, paralysis, mental changes such as forgetfulness and mood swings, depression and epilepsy.

In some embodiments, disclosed herein are methods of decreasing the frequency, severity and/or duration of a relapse of a neurological disease, disorder or condition, the methods comprising administration of a compound or composition described herein. In some embodiments, disclosed herein are methods of decreasing the frequency, severity and/or duration of a relapse of multiple sclerosis, the methods comprising administration of a compound or composition described herein. In some instances, administration of a compound or composition described herein decreases or stops the progression of one or more symptoms in a patient having multiple sclerosis. In some instances, administration of a compound or composition described herein prevents or delays the onset of multiple sclerosis symptoms.

In some instances, a neurological disorder such as multiple sclerosis is treated with a combination of a compound or composition described herein and one or more additional treatments. Addition treatments include, without limitation, plasma exchange, physical therapy, muscle relaxants, exercise, rest, and administration of one or more of the following: corticosteroids, beta interferons, glatiramer acetate, dimethyl fumarate, fingolimod, teriflunomide, natalizumab, mitoxantrone; and combinations thereof.

Neurological diseases, conditions and disorders or characteristics of neurological diseases, conditions and disorders suitable for treatment with a compound or composition described herein include, without limitation, absence of the septum pellucidum, acid lipase disease, acid maltase deficiency, acquired epileptiform aphasia, acute disseminated encephalomyelitis, ADHD, Adie's pupil, Adie's syndrome, adrenoleukodystrophy, agenesis of the corpus callosum, agnosia, aicardi syndrome, neurological complications from AIDS, Alexander disease, Alpers' disease, alternating hemiplegia, Alzheimer's disease, amyotrophic lateral sclerosis (ALS), anencephaly, aneurysm, angelman syndrome, angiomatosis, anoxia, antiphospholipid syndrome, aphasia, apraxia, arachnoid cysts, arachnoiditis, Arnold-Chiari malformation, arteriovenous malformation, Asperger syndrome, ataxia, stroke, Barth syndrome, batten disease, Becker's myotonia, Behcet's disease, Bell's palsy, benign essential blepharospasm, benign focal amyotrophy, benign intracranial hypertension, Bernhardt-Roth syndrome, Binswanger's disease, blepharospasm, Bloch-Sulzberger syndrome, brachial plexus injuries, Bradbury-Eggleston syndrome, brain aneurysm, brain injury, Brown-Sequard syndrome, CADASIL, canavan disease, causalgia, cavernomas, cavernous angioma, cavernous malformation, central cord syndrome, central pain syndrome, central pontine myelinolysis, cephalic disorders, ceramidase deficiency, cerebellar degeneration, cerebellar hypoplasia, cerebral aneurysms, cerebral arteriosclerosis, cerebral atrophy, cerebral beriberi, cerebral cavernous malformation, cerebral gigantism, cerebral hypoxia, cerebral palsy, cerebro-oculo-facio-skeletal syndrome, chiari malformation, chorea, choreoacanthocytosis, chronic inflammatory demyelinating polyneuropathy (CIDP), chronic orthostatic intolerance, cockayne syndrome type II, Coffin Lowry syndrome, colpocephaly, coma, complex regional pain syndrome, congenital facial diplegia, congenital myasthenia, congenital myopathy, congenital vascular cavernous malformations, corticobasal degeneration, cranial arteritis, craniosynostosis, cree encephalitis, Creutzfeldt-Jakob disease, cumulative trauma disorders, Cushing's syndrome, cytomegalic inclusion body disease, cytomegalovirus infection, Dandy-Walker syndrome, Dawson disease, De Morsier's syndrome, Dejerine-Klumpke palsy, dementia, dentate cerebellar ataxia, dentatorubral atrophy, dermatomyositis, developmental dyspraxia, Devic's syndrome, diabetic neuropathy, diffuse sclerosis, dravet syndrome, dysautonomia, dysgraphia, dysphagia, dyspraxia, dyssynergia cerebellaris, dystonias, bulbospinal muscular atrophy, encephalopathy, empty sella syndrome, encephalitis, encephaloceles, encephalotrigeminal angiomatosis, epilepsy, Erb-Duchenne and Dejerine-Klumpke Palsies, Erb's palsy, extrapontine myelinolysis, fabry disease, Fahr's syndrome, familial dysautonomia, familial hemangioma, familial idiopathic basal ganglia calcification, familial periodic paralyses, familial spastic paralysis, Farber's Disease, febrile seizures, fibromuscular dysplasia, fisher syndrome, Friedreich's ataxia, and frontotemporal dementia.

### Inflammation and Inflammatory Disorders

In some embodiments, disclosed herein are methods of treating an inflammatory condition, disease, or disorder with a compound or composition described herein. In some embodiments, disclosed herein are methods of reducing inflammation in a tissue of a subject, the method comprising administering to the subject a compound or composition described herein. In some instances, the tissue is colon tissue.

As used in the present disclosure, “inflammation” refers to the well-known localized response to various types of injury or infection, which is characterized by redness, heat, swelling, and pain, and often also including dysfunction or reduced mobility.

The methods described herein, in some embodiments, include methods for the treatment, reduction of risk, and delaying onset of other inflammatory conditions or diseases with a compound or composition described herein, such as (a) ocular inflammation associated with corneal ulcers, giant papillary conjunctivitis, blepharitis, chelazion, uveitis, dry eye, post-surgical inflammation, and contact lens associated inflammation; (b) allergic diseases such as hay fever, rhinitis, seasonal allergic conjunctivitis, vernal conjunctivitis and other eosinophil-mediated conditions; (c) skin diseases such as psoriasis, contact dermatitis, eczema, infectious skin ulcers, open wounds, and cellulitis; (d) infectious diseases including sepsis, septic shock, encephalitis, infectious arthritis, endotoxic shock, gram negative shock, Jarisch-Herxheimer reaction, shingles, toxic shock, cerebral malaria, bacterial meningitis, acute respiratory distress syndrome (ARDS), lyme disease, and HIV infection; (e) wasting diseases such as cachexia secondary to cancer and HIV; (f) inflammation due to organ, tissue or cell transplantation (e.g., bone marrow, cornea, kidney, lung, liver, heart, skin, pancreatic islets) including transplant rejection, and graft versus host disease; (g) adverse effects from drug therapy, including adverse effects from amphotericin B treatment, adverse effects from immunosuppressive therapy, e.g., interleukin-2 treatment, adverse effects from OKT3 treatment, adverse effects from GM-CSF treatment, adverse effects of cyclosporine treatment, and adverse effects of aminoglycoside treatment, stomatitis, and mucositis due to immunosuppression; (h) cardiovascular conditions including circulatory diseases induced or exasperated by an inflammatory response, such as ischemia, atherosclerosis, peripheral vascular disease, restenosis following angioplasty, inflammatory aortic aneurysm, vasculitis, stroke, spinal cord injury, congestive heart failure, hemorrhagic shock, ischemia/reperfusion injury, vasospasm following subarachnoid hemorrhage, vasospasm following cerebrovascular accident, pleuritis, pericarditis, and the cardiovascular complications of diabetes; (i) dialysis, including pericarditis, due to peritoneal dialysis; (j) gout; and (k) chemical or thermal-induced inflammation due to burns, acid, alkali and the like.

### Autoimmune Diseases

The methods described herein, in some embodiments, include methods for the treatment, reduction of risk, and delaying of onset of an autoimmune disease or disorder with a compound or composition described herein. Examples of autoimmune diseases include, but are not limited to, Alopecia Areata, Lupus, Ankylosing Spondylitis, Meniere's Disease, Antiphospholipid Syndrome, Mixed Connective Tissue Disease, Autoimmune Addison's Disease, Autoimmune Hemolytic Anemia, Myasthenia Gravis, Autoimmune Hepatitis, Pemphigus Vulgaris, Behcet's Disease, Pernicious Anemia, Bullous Pemphigoid, Polyarthritis Nodosa, Cardiomyopathy, Polychondritis, Celiac Sprue-Dermatitis, Polyglandular Syndromes, Chronic Fatigue Syndrome (CFIDS), Polymyalgia Rheumatica, Chronic Inflammatory Demyelinating, Polymyositis and Dermatomyositis, Chronic Inflammatory Polyneuropathy, Primary Agammaglobulinemia, Churg-Strauss Syndrome, Primary Biliary Cirrhosis, Cicatricial Pemphigoid, Psoriasis, CREST Syndrome, Raynaud's Phenomenon, Cold Agglutinin Disease, Reiter's Syndrome, Crohn's Disease, Rheumatic Fever, Discoid Lupus, Multiple Sclerosis, Rheumatoid Arthritis, Essential Mixed, Cryoglobulinemia Sarcoidosis, Fibromyalgia, Scleroderma, Grave's Disease, Sjögren's Syndrome, Guillain-Barre, Stiff-Man Syndrome, Hashimoto's Thyroiditis, Takayasu Arteritis, Idiopathic Pulmonary Fibrosis, Temporal Arteritis/Giant Cell Arteritis, Idiopathic Thrombocytopenia Purpura (ITP), Ulcerative Colitis, IgA Nephropathy, Uveitis, Insulin Dependent Diabetes (Type I), Diabetes (Type II), Vasculitis, Lichen Planus, and Vitiligo.

In some embodiments, the methods for the treatment, reduction of risk, and delaying the onset of an autoimmune disease or disorder further comprise the administration of an immunosuppressant. Immunosuppressants include, without limitation, glucocorticoids, cytostatics, antibodies and drugs that act on immunophilins. Examples of glucocorticoids include cortisol, cortisone, prednisone, prednisolone, methylprednisolone, dexamethasone, betamethasone, triamcinolone, beclometasone, fludrocortisone, deoxycorticosterone, and aldosterone. Examples of cytostatics include alkylating agents (e.g., nitrogen mustards such as cyclophosphamide, nitrosoureas, platinum compounds) and antimetabolites (e.g., folic acid analogues such as methotrexate, purine analogues such as azathioprine and mercaptopurine, pyrimidine analogues such as fluorouracil, protein synthesis inhibitors). Examples of drugs for use in the methods described include ciclosporin, tacrolimus, sirolimus, interferons, opioids, TNF binding proteins, mycophenolate, and fingolimod. Examples of antibodies useful for co-administration with a compound or composition described herein in a method described herein include Antithymocyte globulin, 1D09C3, Adalimumab/D2E7 (Humira; Trudexa), Afelimomab, Afutuzumab/GA101 (type II), Alemtuzumab/Campath-1H (MabCampath), Apolizumab/Hu1D10, Aselizumab, Atlizumab, Basiliximab (Simulect), Bectumomab/IMMU-LL2, Belimumab (Benlysta, LymphoStat-B), Bertilimumab, BL22/CAT-3888, Brentuximab/cAC10/SGN-35, Briakinumab/ABT-874, Canakinumab/ACZ885 (Ilaris), Certolizumab pegol/CDP870 (Cimzia), Clenoliximab, Dacetuzumab/SGN-40, Daclizumab (Zenapax), Eculizumab/5G1.1 (Soliris), Efalizumab (Raptiva, formerly Xanelim), Epratuzumab/hLL2/IMMU-102 (LymphocydeC), Fontolizumab, Fresolimumab/GC-1008, Galiximab/IDEC-114, Gavilimomab/ABX-CBL, Gemtuzumab, Golimumab/CNTO148 (Simponi), HL2434P (IMMU-114), Ibritumomab tiuxetan (MXDPTA)/IDEC Y2B8 (Zevalin), Infliximab/chimeric A2 (cA2) (Remicade), Inolimomab/BT563, Inotuzumab, Keliximab/IDEC CE9.1, Lerdelimumab/CAT-152, Lintuzumab/HuM195 (Zamyl), LMB-2, Lorvotuzumab mertansine, Lumiliximab/IDEC-152, Lym-1 (Oncolym), MDX-060, Mepolizumab/SB-240563, Metelimumab/CAT-192, Mogamulizumab/KW-0761/AMG-761, Moxetumomab pasudotox/CAT-8015/HA22, Muromonab-CD3 (Orthoclone OKT3), Natalizumab (Tysabri, Antegren), Nerelimomab/CDP571, Ocrelizumab/PRO70769 (type I), Odulimomab, Ofatumumab/2F2/HuMax-CD20 (Arzerra) (type I), Omalizumab (Xolair), Otelixizumab/TRX4, Pascolizumab/SB 240683, Reslizumab/SCH 55700 (Cinquil), Rituximab/chimeric 2B8 (IDEC-C2B8) (Rituxan, MabThera) (type I), Ruplizumab (Antova), SAR-3419, Secukinumab/AIN-457, SGN30, Siplizumab/MEDI-507, Teplizumab/MGA031/hOKT3γ1 (Ala-Ala), Tocilizumab (Actemra), Tositumomab (type II), Ustekinumab/CNTO 1275 Vedolizumab/MNL-0002, Veltuzumab/IMMU-106/hA20 (type I), Visilizumab (Nuvion), Zanolimumab/HuMax-CD4, Zolimomab aritox/H65, Abatacept/CTLA4-Ig/BMS-188667 (Orencia), Belatacept/LEA29Y, Atacicept/BLyS/APRIL-Ig, Etanercept/TNFR-Ig (Enbrel), Pegsunercept/pegylated TNFR-Ig, Alefacept (Amevive), and Rilonacept (Arcalyst). Immunosuppressive antibodies include antibodies that target complement-dependent proteins and interleukins.

### Angiogenesis

In some embodiments, described herein are methods for the treatment or prevention of angiogenesis in a subject comprising the administration of a compound or composition described herein to the subject. Angiogenesis includes sprouting angiogenesis and intussusceptive angiogenesis. In some instances, the methods further comprise the administration of an angiogenesis inhibitor, for example, a VEGF inhibitor (bevacizumab), sorafenib sunitinib, pazopanib, everolimus, or a combination thereof.

### Cancer

Autotaxin has been demonstrated increase cell motility, neovascularization, proliferation and aggressiveness of tumors. It is upregulated in numerous tumor lineages, such as breast, renal, liver, glioblastoma, ovarian and prostate cancer.

In some embodiments, disclosed herein are methods of treating cancer with a compound or composition described herein.

Autotaxin is a prometastatic enzyme initially isolated from the conditioned medium of human melanoma cells. In addition, autotaxin overexpression is frequently observed in malignant tumor tissues such as breast cancer, renal cancer, Hodgkin lymphoma, hepatocellular carcinoma, pancreatic cancer and glioblastoma. LPA also contributes to tumorigenesis by increasing motility and invasiveness of cells.

The term “cancer” as used herein, refers to an abnormal growth of cells that tend to proliferate in an uncontrolled way and, in some cases, to metastasize (spread). Types of cancer include, but are not limited to, solid tumors (such as those of the bladder, bowel, brain, breast, endometrium, heart, kidney, lung, liver, uterus, lymphatic tissue (lymphoma), ovary, pancreas or other endocrine organ (thyroid), prostate, skin (melanoma or basal cell cancer) or hematological tumors (such as the leukemias and lymphomas) at any stage of the disease with or without metastases.

Non-limiting examples of cancers include, acute lymphoblastic leukemia, acute myeloid leukemia, adrenocortical carcinoma, anal cancer, appendix cancer, astrocytomas, atypical teratoid/rhabdoid tumor, basal cell carcinoma, bile duct cancer, bladder cancer, bone cancer (osteosarcoma and malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-Cell lymphoma, desmoid tumors, embryonal tumors, endometrial cancer, ependymoblastoma, ependymoma, esophageal cancer, ewing sarcoma family of tumors, eye cancer, retinoblastoma, gallbladder cancer, gastric (stomach) cancer, gastrointestinal carcinoid tumor, gastrointestinal stromal tumor (GIST), gastrointestinal stromal cell tumor, germ cell tumor, glioma, hairy cell leukemia, head and neck cancer, hepatocellular (liver) cancer, Hodgkin lymphoma, hypopharyngeal cancer, intraocular melanoma, islet cell tumors (endocrine pancreas), Kaposi sarcoma, kidney cancer, Langerhans cell histiocytosis, laryngeal cancer, leukemia, Acute lymphoblastic leukemia, acute myeloid leukemia, chronic lymphocytic leukemia, chronic myelogenous leukemia, hairy cell leukemia, liver cancer, non-small cell lung cancer, small cell lung cancer, Burkitt lymphoma, cutaneous T-cell lymphoma, Hodgkin lymphoma, non-Hodgkin lymphoma, lymphoma, Waldenstrom macroglobulinemia, medulloblastoma, medulloepithelioma, melanoma, mesothelioma, mouth cancer, chronic myelogenous leukemia, myeloid leukemia, multiple myeloma, nasopharyngeal cancer, neuroblastoma, non-Hodgkin lymphoma, non-small cell lung cancer, oral cancer, oropharyngeal cancer, osteosarcoma, malignant fibrous histiocytoma of bone, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell (kidney) cancer, retinoblastoma, rhabdomyosarcoma, salivary gland cancer, sarcoma, Sezary syndrome, skin cancer, small cell lung cancer, small intestine cancer, soft tissue sarcoma, squamous cell carcinoma, stomach (gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenstrom macroglobulinemia, and Wilms tumor.

In some embodiments, a compound or composition described herein, or a pharmaceutically acceptable salt thereof, is used in the treatment of ovarian cancer, prostate cancer, breast cancer, lung cancer, melanoma, head and neck cancer, bowel cancer (colorectal cancer), thyroid cancer, glioblastoma, follicular lymphoma, renal cancer, Hodgkin lymphoma, hepatocellular carcinoma, pancreatic cancer or melanoma.

In one embodiment, a compound or composition described herein, or a pharmaceutically acceptable salt thereof, is used in the treatment of pancreatic cancer. In some such embodiments, a compound or composition described herein, or a pharmaceutically acceptable salt thereof is used in combination with one or more chemotherapeutic agent in the treatment of pancreatic cancer.

In some embodiments, a compound or composition described herein disclosed herein, or a pharmaceutically acceptable salt thereof, is used in the treatment of bone metastases.

In some embodiments, a compound or composition described herein disclosed herein, or a pharmaceutically acceptable salt thereof, is used in the treatment of oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, parathyroid cancer, penile cancer, vaginal cancer, thyroid cancer, pancreatic cancer, esophageal cancer, Hodgkin's lymphoma, leukemia-related disorders, mycosis fungoides, or myelodysplastic syndrome.

In some embodiments, a compound or composition described herein disclosed herein, or a pharmaceutically acceptable salt thereof, is used in the treatment of non-small cell lung cancer, pancreatic cancer, breast cancer, ovarian cancer, colorectal cancer, or head and neck cancer.

In some embodiments, a compound or composition described herein, or a pharmaceutically acceptable salt thereof, is used in the treatment of a carcinoma, a tumor, a neoplasm, a lymphoma, a melanoma, a glioma, a sarcoma, or a blastoma.

In some embodiments, the carcinoma is selected from the group consisting of: carcinoma, adenocarcinoma, adenoid cystic carcinoma, adenosquamous carcinoma, adrenocortical carcinoma, well differentiated carcinoma, squamous cell carcinoma, serous carcinoma, small cell carcinoma, invasive squamous cell carcinoma, large cell carcinoma, islet cell carcinoma, oat cell carcinoma, squamous carcinoma, undifferentiatied carcinoma, verrucous carcinoma, renal cell carcinoma, papillary serous adenocarcinoma, merkel cell carcinoma, hepatocellular carcinoma, soft tissue carcinomas, bronchial gland carcinomas, capillary carcinoma, bartholin gland carcinoma, basal cell carcinoma, carcinosarcoma, papilloma/carcinoma, clear cell carcinoma, endometrioid adenocarcinoma, mesothelial, metastatic carcinoma, mucoepidermoid carcinoma, cholangiocarcinoma, actinic keratoses, cystadenoma, and hepatic adenomatosis.

In some embodiments, the tumor is selected from the group consisting of: astrocytic tumors, malignant mesothelial tumors, ovarian germ cell tumor, supratentorial primitive neuroectodermal tumors, Wilm's tumor, pituitary tumors, extragonadal germ cell tumor, gastrinoma, germ cell tumors, gestational trophoblastic tumor, brain tumors, pineal and supratentorial primitive neuroectodermal tumors, pituitary tumor, somatostatin-secreting tumor, endodermal sinus tumor, carcinoids, central cerebral astrocytoma, glucagonoma, hepatic adenoma, insulinoma, medulloepithelioma, plasmacytoma, vipoma, and pheochromocytoma.

In some embodiments, the neoplasm is selected from the group consisting of: intaepithelial neoplasia, multiple myeloma/plasma cell neoplasm, plasma cell neoplasm, interepithelial squamous cell neoplasia, endometrial hyperplasia, focal nodular hyperplasia, hemangioendothelioma, lymphangioleio myomatosis and malignant thymoma.

In some embodiments, the lymphoma is selected from the group consisting of: nervous system lymphoma, AIDS-related lymphoma, cutaneous T-cell lymphoma, non-Hodgkin's lymphoma, mantle cell lymphoma, follicular lymphoma and Waldenstrom's macroglobulinemia.

In some embodiments, the melanoma is selected from the group consisting of: acral lentiginous melanoma, superficial spreading melanoma, uveal melanoma, lentigo maligna melanomas, melanoma, intraocular melanoma, adenocarcinoma nodular melanoma, and hemangioma.

In some embodiments, the sarcoma is selected from the group consisting of: adenomas, adenosarcoma, chondosarcoma, endometrial stromal sarcoma, Ewing's sarcoma, Kaposi's sarcoma, leiomyosarcoma, rhabdomyosarcoma, sarcoma, uterine sarcoma, osteosarcoma, and pseudosarcoma.

In some embodiments, the glioma is selected from the group consisting of: glioma, brain stem glioma, and hypothalamic and visual pathway glioma.

In some embodiments, the blastoma is selected from the group consisting of: pulmonary blastoma, pleuropulmonary blastoma, retinoblastoma, neuroblastoma, medulloblastoma, glioblastoma, and hemangiblastomas.

### Airway Diseases

Inflammatory conditions, diseases, and disorders, which can be treated with a compound or composition described herein, include airway diseases comprising pulmonary inflammation, such as chronic obstructive pulmonary disease (COPD), cystic fibrosis, and asthma. COPD is comprised primarily of two related diseases: chronic bronchitis and emphysema. In both diseases, there is chronic obstruction of the flow of air through the airways and out of the lungs, and the obstruction generally is permanent and progressive over time.

Asthma is a chronic disease of the airways of the lungs, characterized by inflammation and paradoxical narrowing of the bronchi. Asthma includes asthmatic conditions mediated via T-cell action, including extrinsic asthma (allergic asthma), intrinsic asthma (non-allergic asthma), mixed asthma (extrinsic and intrinsic asthma), occupational asthma induced by agents such as toluene diisocyanate, polyvinyl chloride, phthalic anhydride, trimellitic anhydride, plicatic acid (Western Red Cedar trees) or metal salts such as platinum or nickel), drug-induced asthma (including aspirin-induced asthma or asthma induced by non-steroidal anti-inflammatory drugs (NSAIDs)), exercise-induced asthma, and cough variant asthma. In some embodiments, the asthma is an allergic or non-allergic asthmatic condition mediated by T-cell function.

In some embodiments, disclosed herein are methods of treating asthma with a compound or composition described herein. In an asthmatic individual, the release of normal repair mediators, including LPA, is exaggerated or the actions of the repair mediators are inappropriately prolonged leading to inappropriate airway remodeling. Major structural features of the remodeled airway observed in asthma include a thickened Lamina reticularis (the basement membrane-like structure just beneath the airway epithelial cells), increased numbers and activation of myofibroblasts, thickening of the smooth muscle layer, increased numbers of mucus glands and mucus secretions, and alterations in the connective tissue and capillary bed throughout the airway wall. In some embodiments, autotaxin and/or LPA contribute to these structural changes in the airway. In some embodiments, autotaxin and/or LPA are involved in acute airway hyperresponsiveness in asthma. The lumen of the remodeled asthmatic airway is narrower due to the thickening of the airway wall, thus decreasing airflow. In some embodiments, LPA contributes to the long-term structural remodeling and the acute hyperresponsiveness of the asthmatic airway. In some embodiments, LPA contributes to the hyper-responsiveness that is a primary feature of acute exacerbations of asthma.

In some embodiments, disclosed herein are methods of treating or preventing COPD with a compound or composition described herein. The term “chronic obstructive pulmonary disease (COPD)” refers to a group of lung diseases, including chronic bronchitis, emphysema and obliterative bronchiolitis. The most common of these diseases are chronic bronchitis and emphysema. Although a person with COPD may have either chronic bronchitis or emphysema, he or she will often have a mixture of the symptoms of these two conditions. Although emphysema usually results from damage to the lungs caused by environmental insult, usually as a result of long-term smoking, emphysema may also be caused by congenital absence of al-antitrypsin in the lungs; this type of emphysema is usually inherited.

In some embodiments, disclosed herein are methods of treating chronic bronchitis with a compound or composition described herein. Chronic bronchitis (CB) is inflammation of one or more bronchi, usually secondary to infection, and is characterized by excessive production of mucus in the bronchi, accompanied by a recurrent cough which persists for at least three months of the year during at least two successive years. CB is the major non-asthmatic disease of the lung. Many different factors initiate CB, including cigarette smoking, environmental pollution, chronic infections and various genetic abnormalities. Of these factors, cigarette smoking is the most prevalent. Pathological changes in the lung include: (1) hypertrophy and hyperplasia of mucus-secreting glands in the bronchi, (2) increase in goblet cells, (3) disappearance or damage of cilia, and (4) chronic inflammatory changes and narrowing of small airways.

In some embodiments, disclosed herein are methods of treating emphysema with a compound or composition described herein. Emphysema is a lung condition which results from damage to the alveolar sacs in the lungs, usually caused by long-term smoking. This damage leads to a pathological accumulation of air in the tissues.

Administration of LPA in vivo induces airway hyperresponsiveness, itch-scratch responses, infiltration and activation of eosinophils and neutrophils, vascular remodeling, and nociceptive flexor responses. LPA also induces histamine release from mouse and rat mast cells. In an acute allergic reaction, histamine induces various responses, such as contraction of smooth muscle, plasma exudation, and mucus production. Plasma exudation is important in the airway, because the leakage and subsequent airway-wall edema contribute to the development of airway hyperresponsiveness. In some embodiments, disclosed herein are methods of reducing plasma exudation due to an acute allergic reaction with a compound or composition described herein.

### Obesity

In some embodiments, disclosed herein are methods of treating obesity and/or diabetes with a compound or composition described herein.

Autotaxin is responsible for the lysoPLD activity released by adipocytes and exerts a paracrine control on preadipocyte growth via an LPA-dependent mechanism. In addition, autotaxin is up-regulated during adipocyte differentiation and in genetic obesity. In certain instances, autotaxin mRNA is up-regulated in adipocytes from db/db mice suggesting that the up-regulation of autotaxin is related to the severe type 2 diabetes phenotype and adipocyte insulin resistance. In some instances, up-regulation of autotaxin in adipocytes is associated with type 2 diabetes.

The term “obesity,” as used herein, refers to a medical condition in which excess body fat has accumulated to the extent that it may have an adverse effect on health, leading to increased health problems. In some embodiments, “obesity” refers to a weight increase, which is at least 5% of the total body weight. In some embodiments, disclosed herein are methods of treating postmenopausal obesity and/or visceral obesity with a compound or composition described herein.

### Metabolic Disorders

In some embodiments, disclosed herein are methods of treating metabolic disorders, and conditions associated with metabolic disorders, comprising administering a compound or composition described herein. As used herein, a “metabolic disorder” refers to any pathological condition resulting from an alteration in a subject's metabolism. Such disorders include those resulting from an alteration in glucose homeostasis and/or insulin dysfunction. Metabolic disorders, include but are not limited to, metabolic syndrome, elevated blood glucose levels, insulin resistance, glucose intolerance, type 2 diabetes, type 1 diabetes, pre-diabetes, non-alcoholic fatty liver disease, nonalcoholic steatohepatitis, and obesity.

Metabolic disorders are inter-related and can result in disorders across various systems. Addressing the core metabolic disorder can reduce the severity of related conditions in a patient, including, for example, cardiovascular disorders (including, e.g., ischemic heart disease, angina and myocardial infarction, congestive heart failure, high blood pressure, abnormal cholesterol levels, deep vein thrombosis, and pulmonary embolism), neurological disorders (including, e.g., stroke, meralgia paresthetica, migraines, idiopathic, and intracranial hypertension, depression and social stigmatism), rheumatological and orthopedic disorders (including, e.g., gout, poor mobility, osteoarthritis, and lower back pain), dermatological disorders (including, e.g., stretch marks, acanthosis nigricans, lymphedema, cellulitis), gastrointestinal disorders (including, e.g., gastroesophageal reflux disease (GERD) and cholelithiasis (gallstones)), respiratory disorders (including, e.g., obstructive sleep apnea, obesity hypoventilation syndrome, asthma, and increased complications during general anaesthesia), urology and nephrology disorders (including, e.g., erectile dysfunction, urinary incontinence, chronic renal failure, and hypogonadism).

In some embodiments, described herein are methods for treating metabolic disorders. In some embodiments, administering a compound or composition described herein to an individual with a metabolic disorder has a variety of desirable outcomes which include, but are not limited to, reducing blood glucose levels, decreasing plasma lysophosphatidic acid levels, improving insulin sensitivity, increasing insulin secretion, improving glucose tolerance, and decreasing adipose tissue expansion. Any of these outcomes can treat, delay or prevent the onset of a metabolic disorder, wherein such metabolic disorders include, but are not limited to, metabolic syndrome, elevated blood glucose levels, insulin resistance, glucose intolerance, type 2 diabetes, type 1 diabetes, pre-diabetes, non-alcoholic fatty liver disease, nonalcoholic steatohepatitis, and obesity.

In some embodiments, described herein are methods of administering a compound or composition described herein for reducing fasting blood glucose levels in a mouse fed a high fat diet. A mouse fed a high fat diet, as illustrated herein, has higher fasting blood glucose levels than a mouse fed a normal diet. The administration of a compound or composition described herein to a mouse fed a high fat diet reduced fasting blood glucose levels thereby allowing fasting blood glucose levels to approach those levels observed in a mouse fed a normal diet.

In some embodiments, methods disclosed herein comprise administering a compound or composition described herein to a subject with elevated blood glucose levels. In some embodiments, the autotaxin inhibitor is used to treat an underlying metabolic disorder. In some embodiments, the metabolic disorder is treated by reducing blood glucose levels. In some embodiments, the subject is overweight or obese. In some embodiments, the subject has type 2 diabetes. In some embodiments, the subject has non-alcoholic fatty liver disease and/or nonalcoholic steatohepatitis. In some embodiments, the subject does not have a metabolic disorder. In some embodiments, the autotaxin inhibitor delays or prevents the onset of the metabolic disorder by reducing elevated blood glucose levels.

In some embodiments, methods disclosed herein comprise reducing plasma lysophosphatidic acid levels in an individual by administering a compound or composition described herein. In some embodiments, the plasma lysophosphatidic acid levels in the individual are elevated relative to a control. In some embodiments, the control is a person without a metabolic disorder. In some embodiments, the elevated plasma lysophosphatidic acid levels in the individual contribute to or increase the risk for developing a metabolic disorder.

In some embodiments, disclosed herein are methods comprising administering a compound or composition described herein to a subject with elevated plasma lysophosphatidic acid levels relative to a control. In some embodiments, disclosed herein are methods for improving insulin sensitivity comprising administering a compound or composition described herein to an individual sensitive to insulin. In some embodiments, disclosed herein are methods comprising the administration of a compound or composition described herein to a subject with insulin resistance. In some embodiments, disclosed herein are methods comprising the administration of a compound or composition described herein to improve insulin secretion in an individual. In some embodiments, disclosed herein are methods comprising the administration of a compound or composition described herein to improve glucose tolerance in an individual with impaired glucose tolerance. In some embodiments, disclosed herein are methods for decreasing adipose tissue expansion in a subject comprising administering to the subject a compound or composition described herein. In some embodiments, disclosed herein are methods for the treatment of a metabolic disorder in a subject that is overweight or obese comprising administering to the subject a compound or composition described herein.

### Drug Induced Hyperglycemia

In some embodiments, disclosed herein are methods of treating drug induced hyperglycemia in a subject comprising administering to the subject a compound or composition described herein. In some embodiments, administration of a compound or composition described herein to a subject treats, prevents, or ameliorates the symptoms of drug induced hyperglycemia. In some embodiments, administration of a compound or composition described herein to a subject treats, prevents, or ameliorates the symptoms of drug induced hyperglycemia by reducing blood glucose levels. Pharmacological agents can affect glucose homeostasis that can result in hyperglycemia. In some embodiments, the hyperglycemia occurs in the absence of a diagnosis of diabetes. If left untreated, the elevated blood glucose levels can lead to a medical emergency. Symptoms include, but are not limited to fatigue, weakness, fruity odor of the breath, confusion, lack of concentration, shortness of breath, nausea, vomiting, dry skin, and flushing of the skin. Common drug categories that are associated with contributing to hyperglycemia include, but are not limited to: antibiotics, such as fluoroquinolones including gatifloxacin; beta-blockers, such as propranolol, metoprolol or atenolol; thiazide, such as hydrochlorothiazide, and thiazide-like diuretics, and thiazide-like drugs (metolazone); second-generation antipsychotics (SGAs) or “atypical antipsychotics” such as olanzapine or clozapine; corticosteroids; calcinuerin inhibitors such as cyclosporine, sirolimus or tarcrolimus; and protease inhibitors such as ritonavir.

### Stress Induced Hyperglycemia

In some embodiments, disclosed herein are methods of treating stress induced hyperglycemia in a subject comprising administering to the subject a compound or composition described herein. In some embodiments, administration of a compound or composition described herein to a subject treats or prevents or delays the onset of stress induced hyperglycemia. In some embodiments, administration of a compound or composition described herein to a subject treats or prevents or delays the onset of stress induced hyperglycemia by reducing blood glucose levels. Stressed induced hyperglycemia (SIH) is a transient increase in plasma glucose levels higher than 200 mg/dL which occurs during an acute illness or injury. In some embodiments, the hyperglycemia occurs in the absence of a diagnosis of diabetes. The SIH results from an excess of glucose production relative glucose clearance. SIH has been associated with conditions including, but not limited to, myocardial infarction, stroke, and trauma. SIH has been associated with increase mortality and a higher incidence of congestive heart failure and cardiogenic shock in patients after myocardial infarction. Stroke victims have higher mortality associated with SIH and worse odds of desirable neurological outcomes as glucose levels increase with SIH. Hyperglycemia was also shown to be a predictor of infectious complications in the form of pneumonia, urinary tract infections, wound infections and bacteria. Overall, published studies have consistently shown higher morbidity and higher mortality rates in those patients that present with SIH.

### Intraocular Pressure

In some embodiments, disclosed herein are methods of treating elevated intraocular pressure associated with glaucoma in a subject, the methods comprising administration of a compound or composition described herein to the subject.

Glaucoma is one of the leading causes of blindness and is characterized by elevated intraocular pressure (IOP). IOP is a primary risk factor for developing glaucoma and the risk of developing glaucoma decreases when IOP is reduced. Ocular hypotensive therapy is the mainstay of glaucoma treatment. Elevated IOP results from diminished aqueous humor (AH) drainage through the trabecular pathway and autotaxin activity is an abundant protein in human AH. Autotaxin is secreted by human trabecular meshwork cells and autotaxin activity is significantly elevated from glaucoma patients. Inhibition of autotaxin activity in AH by topical and intracameral delivery of a small molecule inhibitor leads to decreased IOP in rabbits.

### Neuropathic Pain

In some embodiments, disclosed herein are methods of treating neuropathic pain with a compound or composition described herein.

LPA induces neuropathic pain as well as demyelination and pain-related protein expression changes via LPA1. In some instances, autotaxin heterozygous knockout mice show about 50% recovery of nerve injury-induced neuropathic pain compared to wild type mice. Lysophosphatidylcholine (LPC), is known to induce neuropathic pain. In certain instances, LPC-induced neuropathic pain is partially reduced in autotaxin heterozygous knockout mice.

Neuropathic pain results from injury to a nerve. In contrast to immediate pain caused by tissue injury, in some embodiments, neuropathic pain develops days or months after a traumatic injury. In addition, neuropathic pain frequently is long-lasting or chronic and can occur spontaneously or as a result of stimulation that normally is not painful.

### EXAMPLES

Additional embodiments are disclosed in further detail in the following examples, which are not in any way intended to limit the scope of the claims.

### X-ray Powder Diffraction

X-ray Powder Diffraction (XRPD) experiments were conducted with PANalytical X'Pert3 and Empyrean X-ray powder diffractometers using the parameters listed below.

| | | |
|---|---|---|
| | Empyrean | X’ Pert³ |
| Parameters | (reflection mode) | (reflection mode) |

| | |
|---|---|
| X-Ray | Cu, Kα, Kα1 (Å): 1.540598; |
| | Kα2 (Å): 1.544426 |
| | Kα2/Kα1 intensity ratio: 0.50 |

| | | |
|---|---|---|
| X-Ray tube setting | 45 kV, 40 mA | 45 kV, 40 mA |
| Divergence slit | Automatic | ⅛° |
| Scan mode | Continuous | Continuous |
| Scan range (2Theta) | 3°~40° | 3°~40° |
| Step size (2Theta) | 0.0167° | 0.0263° |
| Scan step time (s) | 17.78 | 46.67 |
| Test time (min) | ~5.5 min | ~5 min |

### Thermogravimetric Analysis and Differential Scanning Calorimetry

Thermogravimetric analysis (TGA) was conducted using a TA Discovery5500/Q5000 TGA system from TA Instruments. Differential scanning calorimetry (DSC) were conducted using a TA Discovery2500/Q2000 DSC system from TA Instruments. The instrument parameters used for TGA and DSC are listed below.

| Parameters | TGA | DSC |
|---|---|---|
| Method | Ramp | Ramp |
| Sample pan | Aluminum, open | Aluminum, crimped/open |
| Temperature | RT- desired temperature | 25° C. - desired temperature |
| Heating rate | 10° C./min | 10° C./min |
| Purge gas | N₂ | N₂ |

### Molar Ratio Determination

Molar ratios were analyzed with a Waters H-Class ultra-performance liquid chromatography (UPLC) system using the conditions and parameters listed below.

| UPLC | Waters H-class |
|---|---|
| Column | Waters Acquity UPLC BEH C18, |
| | 2.1 mm × 50 mm, 1.7 μm |
| Mobile phase | A: 0.1% TFA in Water |
| | B: 0.1% TFA in ACN |

| | Time (min) | % B |
|---|---|---|
| Gradient table | 0.0 | 20 |
| | 3.0 | 80 |
| | 4.0 | 80 |
| | 4.1 | 20 |
| | 6.0 | 20 |

| | |
|---|---|
| Run time | 6.0 min |
| Post time | 0.0 min |
| Flow rate | 0.5 mL/min |
| Injection volume | 5 μL |
| Detector wavelength | 254 nm |
| Column temp6erature | 40° C. |
| Diluent | Acetonitrile/water (1:1) |

### Stability Measurement

Stability measurements were analyzed with a Waters H-Class ultra-performance liquid chromatography (UPLC) system using the conditions and parameters listed below.

| UPLC | Waters H-class | |
|---|---|---|
| Column | Zorbax SB-C18, 3.0 mm × 150 mm, 3.5 μm | |
| Mobile phase | A: 0.06% TFA in Water: ACN (95:5, v/v) | |
| | B: 0.06% TFA in Water: ACN (5:95, v/v) | |
| | Time (min) | % B |
| Gradient table | 0.0 | 40 |
| | 26.0 | 100 |
| | 26.1 | 40 |
| | 32.0 | 40 |

| | |
|---|---|
| Run time | 32.0 min |
| Post time | 0.0 min |
| Flow rate | 0.45 mL/min |
| Injection volume | 10 μL |
| Detector wavelength | 254 nm |
| Column temp6erature | 35° C. |
| Diluent | Acetonitrile/water (1:1) |

### Ionic Chromatography

Ionic Chromatography (IC) analysis of stoichiometry was performed with a ThermoFisher ICS-1100 instrument using the conditions and parameters listed below.

| Parameter | IC (Thermo ICS1100) | |
|---|---|---|
| Column | Dionex IonpacTM CS12A RFICTM | |
| | 4 × 250 | mm |
| Mobile phase | 25 mM Methanesulfonic acid | |
| Injection volume | 25 | μL |
| Flow rate | 1.0 | mL/min |
| Cell temperature | 35° | C. |
| Column temperature | 35° | C. |
| Current | 80 | mA |

| | |
|---|---|
| Run time | 7 mins for Na⁺, 15 mins for |
| | K⁺, Mg²⁺ |

### Dynamic Vapor Sorption

Dynamic Vapor Sorption (DVS) analysis was conducted using a SMS (Surface Measurement Systems) DVS Intrinsic analyzer using the parameters listed below.

| Parameters | Value | |
|---|---|---|
| Temperature | 25° | C. |
| Sample size | 10-20 | mg |
| Gas and flow rate | N₂, 200 | mL/min |
| dm/dt | 0.002%/ | min |
| Min. dm/dt | 10 | min |
| stability duration | | |
| Max. equilibrium time | 180 | min |

| | |
|---|---|
| RH range | 0% RH~95% RH~0% RH |
| RH step size | 10% (0% RH-90% RH-0% RH) |
| | 5% (90% RH-95% RH and 95% RH-90% RH) |

### ¹H Nuclear Magnetic Resonance (NMR)

The ¹H-NMR spectra were acquired on a Bruker 400 MHz spectrometer. Samples were prepared by dissolving material in DMSO-d₆.

### Example 1: Form A of Compound 1

Initial isolation of Form A of Compound 1 was first performed on a 50 mg scale and then repeated on a 1 g scale. About 50 mg of the sodium salt of Compound 1 was dissolved in about 1 mL MeOH/H₂O (1:1, v/v) at 50° C. The solution was stirred at 50° C. for about 6 hours then a suspension was obtained by adding a 1 M solution of HCl to the solution dropwise. The suspension was stirred for 6 hours at RT and Form A of Compound 1 was isolated by vacuum filtration then washed with water followed by vacuum drying at RT overnight. About 45 mg of Form A of Compound 1 was obtained. The procedure was repeated with about 1 g of the sodium salt of Compound 1 and about 850 mg of Form A of Compound 1 was obtained.

Final isolation of Form A of Compound 1 was performed by dissolving 4.4 g of the sodium salt of Compound 1 in 90 mL of MeOH/H₂O (1:1, v/v) at 50° C. The solution was stirred at 50° C. for about 6 hours and the pH of the solution was measured to be 11.1. A suspension was obtained by adding 10 mL of a 1 M solution of HCl to the solution. The pH of the suspension was measured to be 1.7. A quantity of 840 mg of Form A of Compound that was obtained as described herein was added to the suspension followed by stirring for 6 hours at RT. Form A of Compound 1 was isolated by vacuum filtration then washed with 50 mL water followed by vacuum drying at RT overnight to obtain 4.95 g of Form A of Compound 1. The yield was about 98.8%. This final batch of Form A of Compound 1 was used for screening experiments.

### Example 2: Polymorph Screen Experiments of Compound 1

Polymorph screen experiments using Form A of Compound 1 prepared as described in Example 1 were conducted with various solvents under various conditions. One of the following crystallization methods were used in order to prepare polymorphic forms of Compound 1.

Slurry Experiments at RT: Approximately 20 mg of Form A of Compound 1 was suspended in 0.5 mL of the corresponding solvent in a HPLC vial at room temperature. The suspension was stirred magnetically (˜1000 rpm) for 7 days at room temperature. The resultant solids were isolated for XRPD analysis.

Slurry Experiments at 50° C. Approximately 20 mg of Form A of Compound 1 was suspended in 0.5 mL of the corresponding solvent in a HPLC vial. The suspension was magnetically stirred (˜1000 rpm) for 3 days at 50° C. The resultant solids were isolated for XRPD analysis.

Temperature Cycling: Approximately 20 mg of Form A of Compound 1 was suspended in 0.5 mL of the corresponding solvent in a HPLC vial. The suspension was magnetically stirred (˜1000 rpm) for 3 days with temperature cycling from 50° C. to 5° C. (0.1° C./min, three cycles). The resultant solids were isolated for XRPD analysis.

Antisolvent Precipitation: Approximately 20 mg Form A of Compound 1 was dissolved in minimal solvent at a specified temperature. The solution was filtered to obtain a clear solution and the solution was magnetically stirred (˜1000 rpm), followed by the slow addition of anti-solvent until either precipitate appeared, or the total volume of anti-solvent reached 5 mL. The obtained precipitate was isolated for XRPD analysis.

The results of the polymorph screen experiments produced five crystalline forms, which were designated as Form A, Form B, Form C, Form D, and Form E. Form E was obtained by nitrogen purging of Form A and transferred back to Form A after 5 hours at ambient surroundings. The experimental results for Form A, Form B, Form C, and Form D are shown in Table 1.

TABLE 1

XRPD^{b}
Pattern

Slurry at RT Solvent^{a} (v/v)

| | |
|---|---|
| ACN/H₂O (995:5, a_{w} = 0.2) | Form C |
| ACN/H₂O (987:13, a_{w} = 0.4) | Form C |
| ACN/H₂O (976:24, a_{w} = 0.6) | Form A |
| ACN/H₂O (955:45, a_{w} = 0.8) | Form A |
| ACN | Form C |
| H₂O | Form A |
| IPA | Form C |
| IPAc | Form A |
| DCM | Form C + |
| | Form A |
| MIBK | Form A |

Slurry at 50° C. Solvent^{a} (v/v)

| | |
|---|---|
| MIBK | Form A* |
| CHCl₃ | Form D |
| IPAc | Form A* |
| IPA | Form C |

Temperature Cycling Solvent^{a} (v/v)

| | |
|---|---|
| ACN/H₂O (1:4) | LC † |
| EtOH/H₂O (1:4) | LC † |
| 1,4-Dioxane/H₂O (1:4) | No solid |
| | obtained † |
| Acetone/Toluene (1:4) | Form A |
| EtOAc/Toluene (1:4) | Form A |
| THF/n-Heptane (1:4) | LC † |

| | |
|---|---|
| Antisolvent | Solvent^{a} (v/v) |

| | | |
|---|---|---|
| H₂O | THF | Form A + |
| | | a.pk |
| | Acetone | Form C |
| | MeOH | Form C |
| | DMSO | Form A |
| n-Heptane | CHCl₃ | Form B |
| | EtOH | Form A |
| | EtOAc | Form A |
| | 1,4-Dioxane | Form A |
| Toluene | ACN | Form A†† |
| | MTBE | Form A†† |

*Clear solution was obtained at 50° C. and the sample was obtained after cooling at 5° C.
†Clear solution was obtained and the sample was obtained after evaporation at RT.
††Clear solution was obtained and solid was obtained after evaporation.
^{a}a_{w} = water activity; ACN = acetonitrile; CHCl₃ = chloroform; DCM = dichloromethane; DMSO = dimethylsulfoxide; EtOH = ethanol; EtOAc = ethyl acetate; IPA = isopropyl alcohol; IPAc = isopropyl acetate; MeOH = methanol; MIBK = methyl isobutyl ketone; MTBE = methyl tert-butyl ether; THF = tetrahydrofuran;
^{b}LC = low crystallinity; a.pk = additional peaks

### Example 3: Competitive Slurry Experiments for Crystalline Forms of Compound 1

Solutions (˜2 mL) of acetone/H₂O systems with various water activities (including pure acetone and H₂O) were saturated with Form A of Compound 1. Once saturated, equal amounts (approximately 10 mg each) of Form A and Form C were suspended in the saturated solutions and the samples were then stirred magnetically at room temperature for 2 days. The obtained solids were isolated for XRPD analysis. The results of the interconversion slurry studies are shown in Table 2.

TABLE 2

| Starting Material | Solvent System | Water Activity | XRPD |
|---|---|---|---|
| Crystal Forms | (v:v) | (a_{w}) | Result |
| Form A + | Acetone | 0.0 | Form C |
| Form C | Acetone/H₂O (986:14) | 0.2 | Form C |
| | Acetone/H₂O (950:50) | 0.4 | Form C |
| | Acetone/H₂O (858:142) | 0.6 | Form A |
| | Acetone/H₂O (605:395) | 0.8 | Form A |
| | H₂O | 1.0 | Form A |

### Example 4: Salt Screen Experiments for Crystalline Forms of Compound 1

Salt screen experiments using Compound 1 and one equivalent of counter ion were conducted with various solvents and stirred at RT for 4 days. Slurry at 5° C., anti-solvent addition and evaporation were applied to clear solutions to induce solid formation. After centrifugation, resulting solids were dried under vacuum at RT for overnight, and then analyzed by XRPD. The XRPD results of the salt screen experiments are shown in Table 3.

TABLE 3

| | | Acetone/H₂O | | |
|---|---|---|---|---|
| Counter ion | EtOH | (19:1, v/v) | EtOAc | ACN |
| No Counter Ion | Form B | Form A* | Form A* | Form C |
| L- arginine | Form 1 of | Form 2 of | Form 1 of | Form 2 of |
| | Compound 1-A | Compound 1-A | Compound 1-A | Compound 1-A + |
| | | | | L- arginine |
| N- | Form 3 of | Form 4 of | Form 3 of | Form 3 of |
| methylglucamine | Compound 1-B | Compound 1-B | Compound 1-B | Compound 1-B |
| NaOH | Na salt | Na salt Form A | Na salt Form B | Na salt |
| | Form A + | | | Form B + |
| | Form B | | | Form C |
| KOH | K salt Form A | K salt Form A | K salt Form A | K salt Form A |
| Choline | Choline salt | Form A* | Choline salt Form | Choline salt Form |
| | Form B | | A* | A* |
| Mg(OH)₂ | Form B + | Mg salt Form A | Mg salt | Mg salt |
| | a.pk | | Form A + | Form A + |
| | | | Mg(OH)₂ | Form C |
| L-Lysinc | Form B | L-Lysine salt | Amorphous | Form C |
| | | Form A | | |
| Ammonia | Form B | Form A* | Form A | Form C |
| Betaine | Form B | A* | Betaine salt A | Betaine salt A |
| TMA | TMA salt Form A | TMA salt | TMA salt Form A | Form C + |
| | | Form A + | | TMA |
| | | one peak* | | |
| L-Proline | Form B | Form A* | L-Proline | L-Proline |
| | | | salt Form A | salt Form A |
| DEA | DEA salt Form Af | Form At | DEA salt Form At | LC * |
| Zn(OH)₂ | LC | LC | LC | Form C |
| Ca(OH)₂ | Form B + | Ca salt Form A | Form C + | Form C + |
| | Ca(OH)₂ | | onc pcak | Ca(OH)₂ |

*The sample was obtained after evaporation.
†The sample was obtained after anti-solvent addition.
ACN = acetonitrile;
EtOH = ethanol;
EtOAc = ethyl acetate
DEA = diethylamine;
TMA = tromethamine
LC = low crystallinity;
a.pk = additional peaks

### Example 5: Preparation of Forms 1 and 2 of Compound 1-A

To a 5 mL glass vial was added Compound 1 Form A (298.8 mg), 108.0 mg of L-arginine and 2.0 mL EtOH. The resultant suspension was stirred magnetically (1000 rpm) at room temperature for 7 days. The suspension was then centrifuged to isolate the solid that was dried under vacuum at room temperature for approximately 8 hours. ¹H NMR spectroscopy of crystalline Form 1 of Compound 1-A exhibited no EtOH present and a 1.0:1.0 stoichiometry of Compound 1 to L-arginine. The DVS plot of Form 1 of Compound 1-A showed a moisture uptake of 0.32% at 25° C./80% RH, indicating that Form 1 of Compound 1-A was slightly hygroscopic. No form change was observed after DVS test.

To a 5 mL glass vial was added Compound 1 Form A (300 mg), 1.0 equivalent L-arginine and 2 mL acetone/water (19:1, v/v). The resultant suspension was stirred magnetically (1000 rpm) at room temperature for 6 days. The suspension was then centrifuged to isolate the solid that was dried under vacuum at room temperature for approximately 8 hours. ¹H NMR spectroscopy of crystalline Form 2 of Compound 1-A exhibited no acetone present and a 1:1 stoichiometry of Compound 1 to L-Arginine. TGC/DSC results of Form 2 of Compound 1-A showed a weight loss of 1.3% up to 200° C.

Competitive slurry experiments for Form 1 and Form 2 of Compound 1-A were conducted by preparing saturated solutions (˜2 mL) of the Form 1 of Compound 1-A in either EtOH or acetone. Once saturated, equal masses of Form 1 of Compound 1-A and Form 2 of Compound 1-A were suspended in the saturated solutions and the samples were then stirred magnetically at room temperature at or 50° C. The obtained solids were isolated for XRPD analysis. The results of the interconversion slurry studies are shown in Table 4.

TABLE 4

| Starting Form | Solvent | Temperature | XRPD Results |
|---|---|---|---|
| Form 1 of | EtOH | RT | Form 2 of |
| Compound 1-A + | | | Compound 1-A |
| Form 2 of | Acetone | | Form 2 of |
| Compound 1-A | | | Compound 1-A |
| | EtOH | 50° C. | Form 2 of |
| | | | Compound 1-A |
| | Acetone | | Form 2 of |
| | | | Compound 1-A |

### Example 6: Preparation of Forms 3 and 4 of Compound 1-B

To a 5 mL glass vial was added Compound 1 Form A (301.9 mg), 121.1 mg of N-methylglucamine and 2.0 mL ACN. The resultant suspension was stirred magnetically (1000 rpm) at room temperature for 7 days. The suspension was then centrifuged to isolate the solid that was dried under vacuum at room temperature for approximately 8 hours. 1H NMR spectroscopy of crystalline Form 3 of Compound 1-B exhibited no ACN present and a 1.0:1.0 stoichiometry of Compound 1 to N-methylglucamine. The DVS plot of Form 3 of Compound 1-B showed a moisture uptake of 0.1% at 25° C./80% RH, indicating that Form 3 of Compound 1-B was non-hygroscopic. No form change was observed after DVS test.

To a 5 mL glass vial was added Compound 1 Form A (300 mg), 1.0 equivalent N-methylglucamine and 2 mL acetone/water (19:1, v/v). The resultant suspension was stirred magnetically (1000 rpm) at room temperature for 4 days. The suspension was then centrifuged to isolate the solid that was dried under vacuum at room temperature for approximately 8 hours. ¹H NMR spectroscopy of crystalline Form 4 of Compound 1-B exhibited no acetone present and a 1:1.2 stoichiometry of Compound 1 to N-methylglucamine. TGC/DSC results of Form 4 of Compound 1-B showed a weight loss of 0.9% up to 150° C.

Competitive slurry experiments for Form 3 and Form 4 of Compound 1-B were conducted by preparing a saturated solution (˜2 mL) of Form 3 of Compound 1-B in acetone. Once saturated, equal masses of Form 3 of Compound 1-B and Form 4 of Compound 1-B were suspended in the saturated solution and the samples were then stirred magnetically at room temperature at or 50° C. The obtained solids were isolated for XRPD analysis. The results of the interconversion slurry studies are shown in Table 5.

TABLE 5

| | | | XRPD |
|---|---|---|---|
| Starting Form | Solvent | Temperature | Results |
| Form 3 of | Acetone | RT | Form 3 of |
| Compound 1-B + | | | Compound 1-B |
| Form 4 of | Acetone | 50° C. | Form 3 of |
| Compound 1-B | | | Compound 1-B |

### Example 7: Characterization of Crystalline Samples

The crystalline solid forms were characterized by a combinations of XRPD, thermogravimetry (TG), DSC and/or solution NMR.

### Crystalline Form A of Compound 1

The XRPD results of crystalline Form A (FIG. 1) show good crystallinity. A melting temperature at approximately 183° C. was observed using differential scanning calorimetry (FIG. 2).

XRPD measurements of crystalline Form A of Compound 1 were measured. Observed peaks are shown in Table 6. Prominent peaks are listed in Table 7.

TABLE 6

| Diffraction angle | d space | Intensity |
|---|---|---|
| °2θ (deg) | (Å) | (%) |
| 7.2 | 12.2 | 100.0 |
| 10.4 | 8.5 | 7.6 |
| 12.0 | 7.4 | 2.0 |
| 14.5 | 6.1 | 2.3 |
| 17.9 | 5.0 | 5.8 |
| 21.5 | 4.1 | 8.2 |
| 21.8 | 4.1 | 6.9 |
| 22.3 | 4.0 | 5.0 |
| 23.5 | 3.8 | 1.9 |
| 25.0 | 3.6 | 2.8 |
| 25.8 | 3.5 | 4.6 |
| 27.2 | 3.3 | 3.2 |
| 27.7 | 3.2 | 1.8 |
| 29.8 | 3.0 | 1.6 |
| 31.6 | 2.8 | 1.8 |
| 36.7 | 2.5 | 1.6 |

TABLE 7

| Diffraction angle | d space | Intensity |
|---|---|---|
| °2θ (deg) | (Å) | (%) |
| 7.2 | 12.2 | 100.0 |
| 10.4 | 8.5 | 7.6 |
| 17.9 | 5.0 | 5.8 |
| 21.5 | 4.1 | 8.2 |
| 21.8 | 4.1 | 6.9 |
| 25.8 | 3.5 | 4.6 |

### Crystalline Form B of Compound 1

The XRPD results of crystalline Form B (FIG. 3) show good crystallinity. A melting temperature at approximately 203° C. was observed using differential scanning calorimetry (FIG. 4).

XRPD measurements of crystalline Form B of Compound 1 were measured. Observed peaks are shown in Table 8. Prominent peaks are listed in Table 9.

TABLE 8

| Diffraction angle | d space | Intensity |
|---|---|---|
| °2θ (deg) | (Å) | (%) |
| 6.1 | 14.5 | 100.0 |
| 7.1 | 12.5 | 63.5 |
| 8.8 | 10.1 | 7.7 |
| 10.5 | 8.4 | 11.0 |
| 12.1 | 7.3 | 5.6 |
| 14.0 | 6.3 | 16.5 |
| 14.4 | 6.1 | 35.3 |
| 15.0 | 5.9 | 24.6 |
| 17.0 | 5.2 | 20.4 |
| 18.3 | 4.9 | 22.3 |
| 21.1 | 4.2 | 41.1 |
| 21.9 | 4.1 | 25.2 |
| 23.1 | 3.9 | 32.3 |
| 24.1 | 3.7 | 18.6 |
| 24.4 | 3.6 | 15.6 |
| 25.4 | 3.5 | 33.7 |
| 27.0 | 3.3 | 16.2 |
| 27.5 | 3.2 | 1.4 |
| 28.2 | 3.2 | 15.9 |

TABLE 9

| Diffraction angle | d space | Intensity |
|---|---|---|
| °2θ (deg) | (Å) | (%) |
| 6.1 | 14.5 | 100.0 |
| 7.1 | 12.5 | 63.5 |
| 8.8 | 10.1 | 7.7 |
| 14.4 | 6.1 | 35.3 |
| 17.0 | 5.2 | 20.4 |
| 18.3 | 4.9 | 22.3 |
| 21.1 | 4.2 | 41.1 |
| 21.9 | 4.1 | 25.2 |
| 23.1 | 3.9 | 32.3 |
| 25.4 | 3.5 | 33.7 |
| 28.2 | 3.2 | 15.9 |

### Crystalline Form C of Compound 1

The XRPD results of crystalline Form C (FIG. 5) show good crystallinity. A melting temperature at approximately 204° C. was observed using differential scanning calorimetry (FIG. 6).

XRPD measurements of crystalline Form C of Compound 1 were measured. Observed peaks are shown in Table 10. Prominent peaks are listed in Table 11.

TABLE 10

| Diffraction angle | d space | Intensity |
|---|---|---|
| °2θ (deg) | (Å) | (%) |
| 6.7 | 13.22 | 100.0 |
| 9.6 | 9.2 | 3.1 |
| 11.2 | 7.9 | 5.9 |
| 12.2 | 7.2 | 2.3 |
| 13.3 | 6.6 | 6.3 |
| 16.1 | 5.5 | 10.3 |
| 17.7 | 5.0 | 5.3 |
| 18.1 | 4.9 | 6.2 |
| 18.9 | 4.7 | 6.7 |
| 19.3 | 4.6 | 1.4 |
| 20.0 | 4.4 | 14.0 |
| 20.6 | 4.3 | 7.2 |
| 21.5 | 4.1 | 8.6 |
| 24.5 | 3.6 | 3.6 |
| 24.9 | 3.6 | 6.4 |
| 25.4 | 3.5 | 1.7 |
| 26.5 | 3.4 | 2.3 |
| 27.5 | 3.2 | 3.8 |
| 28.0 | 3.2 | 2.0 |
| 28.3 | 3.2 | 3.4 |
| 30.4 | 2.9 | 2.1 |
| 33.7 | 2.7 | 4.6 |
| 33.9 | 2.6 | 3.2 |
| 37.2 | 2.4 | 1.5 |

TABLE 11

| Diffraction angle | | Intensity |
|---|---|---|
| °2θ (deg) | d space (Å) | (%) |
| 6.7 | 13.22 | 100.0 |
| 11.2 | 7.9 | 5.9 |
| 13.3 | 6.6 | 6.3 |
| 16.1 | 5.5 | 10.3 |
| 17.7 | 5.0 | 5.3 |
| 18.1 | 4.9 | 6.2 |
| 18.9 | 4.7 | 6.7 |
| 19.3 | 4.6 | 1.4 |
| 20.0 | 4.4 | 14.0 |
| 20.6 | 4.3 | 7.2 |
| 21.5 | 4.1 | 8.6 |
| 24.9 | 3.6 | 6.4 |

### Crystalline Form D of Compound 1

The XRPD results of crystalline Form D (FIG. 7) show good crystallinity. A melting temperature at approximately 203° C. was observed using differential scanning calorimetry (FIG. 8).

XRPD measurements of crystalline Form D of Compound 1 were measured. Observed peaks are shown in Table 12. Prominent peaks are listed in Table 13.

TABLE 12

| Diffraction angle | | Intensity |
|---|---|---|
| °2θ (deg) | d space (Å) | (%) |
| 6.7 | 13.2 | 20.4 |
| 11.2 | 7.9 | 34.1 |
| 12.0 | 7.4 | 28.4 |
| 13.7 | 6.4 | 12.7 |
| 16.0 | 5.5 | 37.4 |
| 17.5 | 5.1 | 100.0 |
| 17.9 | 5.0 | 30.8 |
| 18.7 | 4.7 | 67.7 |
| 20.0 | 4.4 | 14.3 |
| 20.4 | 4.3 | 75.4 |
| 21.4 | 4.2 | 50.0 |
| 22.9 | 3.9 | 19.5 |
| 24.1 | 3.7 | 36.1 |
| 24.7 | 3.6 | 80.5 |
| 25.1 | 3.5 | 14.1 |
| 25.7 | 3.5 | 10.5 |
| 26.1 | 3.4 | 35.1 |
| 27.3 | 3.3 | 14.4 |
| 27.7 | 3.2 | 21.8 |
| 28.3 | 3.2 | 7.1 |
| 28.8 | 3.1 | 3.8 |
| 29.9 | 3.0 | 17.8 |
| 30.3 | 3.0 | 7.9 |
| 30.8 | 2.9 | 11.7 |
| 33.8 | 2.7 | 10.3 |
| 34.3 | 2.6 | 7.4 |
| 35.4 | 2.5 | 7.2 |
| 38.1 | 2.4 | 2.5 |

TABLE 13

| Diffraction angle | | Intensity |
|---|---|---|
| °2θ (deg) | d space (Å) | (%) |
| 6.7 | 13.2 | 20.4 |
| 11.2 | 7.9 | 34.1 |
| 12.0 | 7.4 | 28.4 |
| 13.7 | 6.4 | 12.7 |
| 16.0 | 5.5 | 37.4 |
| 17.5 | 5.1 | 100.0 |
| 17.9 | 5.0 | 30.8 |
| 18.7 | 4.7 | 67.7 |
| 20.4 | 4.3 | 75.4 |
| 21.4 | 4.2 | 50.0 |
| 22.9 | 3.9 | 19.5 |
| 24.1 | 3.7 | 36.1 |
| 24.7 | 3.6 | 80.5 |
| 29.9 | 3.0 | 17.8 |
| 30.3 | 3.0 | 7.9 |
| 30.8 | 2.9 | 11.7 |

### Crystalline Form E of Compound 1

The XRPD results of crystalline Form E (FIG. 9) show poor crystallinity. XRPD measurements of crystalline Form E of Compound 1 were measured. Observed peaks are shown in Table 14. Prominent peaks are listed in Table 15.

TABLE 14

| Diffraction angle | | Intensity |
|---|---|---|
| °2θ (deg) | d space (Å) | (%) |
| 6.7 | 13.2 | 100.0 |
| 7.3 | 12.1 | 9.9 |
| 9.9 | 8.9 | 12.4 |
| 12.0 | 7.4 | 10.1 |
| 13.3 | 6.7 | 10.2 |
| 15.0 | 5.9 | 6.9 |
| 17.3 | 5.1 | 43.6 |
| 18.6 | 4.8 | 13.4 |
| 19.9 | 4.5 | 35.4 |
| 20.5 | 4.3 | 17.8 |
| 21.3 | 4.2 | 8.5 |
| 23.2 | 3.8 | 13.6 |
| 23.8 | 3.7 | 6.6 |
| 24.6 | 3.6 | 15.5 |
| 25.8 | 3.5 | 14.7 |
| 26.3 | 3.4 | 20.9 |
| 26.7 | 3.3 | 11.5 |
| 28.3 | 3.2 | 5.8 |
| 29.2 | 3.1 | 14.3 |
| 29.8 | 3.0 | 15.8 |
| 31.6 | 2.8 | 8.7 |
| 33.5 | 2.7 | 5.5 |
| 35.0 | 2.6 | 9.3 |
| 38.7 | 2.3 | 8.1 |

TABLE 15

| Diffraction angle | | Intensity |
|---|---|---|
| °2θ (deg) | d space (Å) | (%) |
| 6.7 | 13.2 | 100.0 |
| 9.9 | 8.9 | 12.4 |
| 12.0 | 7.4 | 10.1 |
| 13.3 | 6.7 | 10.2 |
| 17.3 | 5.1 | 43.6 |
| 18.6 | 4.8 | 13.4 |
| 19.9 | 4.5 | 35.4 |
| 23.2 | 3.8 | 13.6 |
| 24.6 | 3.6 | 15.5 |
| 26.3 | 3.4 | 20.9 |
| 29.2 | 3.1 | 14.3 |
| 29.8 | 3.0 | 15.8 |
| 35.0 | 2.6 | 9.3 |
| 38.7 | 2.3 | 8.1 |

### Crystalline Form 1 of Compound 1-A

The XRPD results of crystalline Form 1 of Compound 1-A (FIG. 10) show good crystallinity. A melting temperature at approximately 227° C. was observed using differential scanning calorimetry (FIG. 11).

XRPD measurements of crystalline Form 1 of Compound 1-A were measured. Observed peaks are shown in Table 16. Prominent peaks are listed in Table 17.

TABLE 16

| Diffraction angle | | Intensity |
|---|---|---|
| °2θ (deg) | d space (Å) | (%) |
| 5.9 | 15.0 | 75.0 |
| 8.8 | 10.0 | 91.6 |
| 10.4 | 8.5 | 100.0 |
| 12.3 | 7.2 | 39.6 |
| 14.6 | 6.1 | 86.3 |
| 17.1 | 5.2 | 28.1 |
| 17.5 | 5.1 | 33.1 |
| 19.4 | 4.6 | 25.1 |
| 20.7 | 4.3 | 15.4 |
| 22.0 | 4.0 | 8.6 |
| 22.4 | 4.0 | 8.0 |
| 23.8 | 3.7 | 4.2 |
| 25.7 | 3.5 | 19.0 |
| 27.0 | 3.3 | 11.7 |
| 28.1 | 3.2 | 10.9 |
| 29.6 | 3.0 | 9.1 |

TABLE 17

| Diffraction angle | | Intensity |
|---|---|---|
| °2θ (deg) | d space (Å) | (%) |
| 5.9 | 15.0 | 75.0 |
| 8.8 | 10.0 | 91.6 |
| 10.4 | 8.5 | 100.0 |
| 12.3 | 7.2 | 39.6 |
| 14.6 | 6.1 | 86.3 |
| 17.1 | 5.2 | 28.1 |
| 17.5 | 5.1 | 33.1 |
| 19.4 | 4.6 | 25.1 |

### Crystalline Form 2 of Compound 1-A

The XRPD results of crystalline Form 2 of Compound 1-A (FIG. 13) show good crystallinity. A melting temperature at approximately 229° C. was observed using differential scanning calorimetry (FIG. 14).

XRPD measurements of crystalline Form 2 of Compound 1-A were measured. Observed peaks are shown in Table 18. Prominent peaks are listed in Table 19.

TABLE 18

| Diffraction angle | | Intensity |
|---|---|---|
| °2θ (deg) | d space (Å) | (%) |
| 5.7 | 15.4 | 83.2 |
| 8.6 | 10.3 | 86.9 |
| 9.2 | 9.7 | 16.2 |
| 10.3 | 8.6 | 75.4 |
| 10.5 | 8.4 | 66.9 |
| 12.1 | 7.3 | 28.5 |
| 12.4 | 7.2 | 36.6 |
| 14.2 | 6.2 | 100.0 |
| 14.5 | 6.1 | 49.2 |
| 16.6 | 5.4 | 26.5 |
| 16.9 | 5.3 | 24.4 |
| 17.8 | 5.0 | 34.4 |
| 19.3 | 4.6 | 30.3 |
| 20.0 | 4.4 | 19.7 |
| 20.5 | 4.3 | 28.7 |
| 21.1 | 4.2 | 9.9 |
| 21.8 | 4.1 | 24.4 |
| 24.4 | 3.7 | 24.1 |
| 25.1 | 3.6 | 37.0 |
| 25.6 | 3.5 | 56.4 |
| 26.2 | 3.4 | 9.6 |
| 27.0 | 3.3 | 26.5 |
| 28.5 | 3.1 | 11.3 |
| 31.6 | 2.8 | 9.6 |
| 35.4 | 2.5 | 7.5 |

TABLE 19

| Diffraction angle | | Intensity |
|---|---|---|
| °2θ (deg) | d space (Å) | (%) |
| 5.7 | 15.4 | 83.2 |
| 8.6 | 10.3 | 86.9 |
| 10.3 | 8.6 | 75.4 |
| 10.5 | 8.4 | 66.9 |
| 12.1 | 7.3 | 28.5 |
| 12.4 | 7.2 | 36.6 |
| 14.2 | 6.2 | 100.0 |
| 14.5 | 6.1 | 49.2 |
| 16.6 | 5.4 | 26.5 |
| 16.9 | 5.3 | 24.4 |

### Crystalline Form 3 of Compound 1-B

The XRPD results of crystalline Form 3 of Compound 1-B (FIG. 15) show good crystallinity. A melting temperature at approximately 165° C. was observed using differential scanning calorimetry (FIG. 16).

XRPD measurements of crystalline Form 3 of Compound 1-B were measured. Observed peaks are shown in Table 20. Prominent peaks are listed in Table 21.

TABLE 20

| Diffraction angle | | Intensity |
|---|---|---|
| °2θ (deg) | d space (Å) | (%) |
| 8.3 | 10.7 | 60.7 |
| 9.5 | 9.3 | 41.6 |
| 10.5 | 8.4 | 100.0 |
| 10.8 | 8.2 | 97.7 |
| 12.2 | 7.3 | 19.7 |
| 12.5 | 7.1 | 33.2 |
| 14.2 | 6.3 | 30.1 |
| 14.5 | 6.1 | 18.3 |
| 16.4 | 5.4 | 10.0 |
| 16.8 | 5.3 | 11.8 |
| 18.4 | 4.8 | 64.2 |
| 18.6 | 4.8 | 71.2 |
| 19.0 | 4.7 | 14.7 |
| 20.3 | 4.4 | 13.6 |
| 21.8 | 4.1 | 4.6 |
| 24.2 | 3.7 | 7.2 |
| 25.4 | 3.5 | 10.0 |
| 27.8 | 3.2 | 3.2 |
| 30.7 | 2.9 | 4.8 |

TABLE 21

| Diffraction angle | | Intensity |
|---|---|---|
| °2θ (deg) | d space (Å) | (%) |
| 8.3 | 10.7 | 60.7 |
| 9.5 | 9.3 | 41.6 |
| 10.5 | 8.4 | 100.0 |
| 10.8 | 8.2 | 97.7 |
| 12.2 | 7.3 | 19.7 |
| 12.5 | 7.1 | 33.2 |
| 14.2 | 6.3 | 30.1 |
| 14.5 | 6.1 | 18.3 |
| 16.4 | 5.4 | 10.0 |
| 16.8 | 5.3 | 11.8 |
| 18.6 | 4.8 | 71.2 |

### Crystalline Form 4 of Compound 1-B

The XRPD results of crystalline Form 4 of Compound 1-B (FIG. 18) show good crystallinity. A melting temperature at approximately 159° C. was observed using differential scanning calorimetry (FIG. 19).

XRPD measurements of crystalline Form 4 of Compound 1-B were measured. Observed peaks are shown in Table 22. Prominent peaks are listed in Table 23.

TABLE 22

| Diffraction angle | | Intensity |
|---|---|---|
| °2θ (deg) | d space (Å) | (%) |
| 8.5 | 10.4 | 19.5 |
| 9.6 | 9.2 | 100.0 |
| 11.6 | 7.6 | 75.7 |
| 14.1 | 6.3 | 10.4 |
| 16.1 | 5.5 | 13.5 |
| 16.6 | 5.3 | 46.0 |
| 17.9 | 5.0 | 5.7 |
| 19.1 | 4.6 | 34.5 |
| 19.4 | 4.6 | 36.0 |
| 19.7 | 4.5 | 15.1 |
| 20.8 | 4.3 | 2.8 |
| 22.5 | 4.0 | 5.2 |
| 23.1 | 3.9 | 6.6 |
| 24.2 | 3.7 | 20.2 |
| 25.4 | 3.5 | 23.8 |
| 27.4 | 3.3 | 2.8 |
| 28.6 | 3.1 | 7.1 |
| 31.0 | 2.9 | 8.1 |
| 32.6 | 2.8 | 2.7 |

TABLE 23

| Diffraction angle | | Intensity |
|---|---|---|
| °2θ (deg) | d space (Å) | (%) |
| 8.5 | 10.4 | 19.5 |
| 9.6 | 9.2 | 100.0 |
| 11.6 | 7.6 | 75.7 |
| 16.1 | 5.5 | 13.5 |
| 16.6 | 5.3 | 46.0 |
| 19.1 | 4.6 | 34.5 |
| 19.4 | 4.6 | 36.0 |
| 24.2 | 3.7 | 20.2 |
| 25.4 | 3.5 | 23.8 |

### Example 8: Aqueous Solubility and Crystallization Form Characterization

Equilibrium solubility of Form A, Form C, Form 1 of Compound 1-A, and Form 3 of Compound 1-B was measured in water at ambient temperature. A known volume of water was placed in a 1-dram glass vial and weight aliquots of the sample were added while magnetically stirring at room temperature. If the solid dissolved, another aliquot of solvent was added. The solubility and characterization data of Form A, Form B, Form C, Form D, Form 1 of Compound 1-A, and Form 3 of Compound 1-B are shown in Table 24.

TABLE 24

| | TGA Weight | Endotherm in | | Solubility in |
|---|---|---|---|---|
| | Loss | DSC | | H₂O at 37° C. |
| Form (ID) | (wt %, temp.) | (peak ° C.) | Form ID | (24 h, mg/mL) |
| Form A | 4.3 (110° C.) | 108.5, 133.4, | Hydrate | 0.003 |
| | | 153.3 (exothermic | | |
| | | peak), 182.9 | | |
| Form B | 4.0 (180° C.) | 203.2 | EtOH | not tested |
| | | | solvate | |
| Form C | 1.6 (180° C.) | 203.4 | Anhydrate | 0.002 |
| Form D | 10.5 (220° C.) | 202.5 | CHCl₃ | not tested |
| | | | solvate | |
| Form E | Freeform Type E transferred to freeform Type A after | | | |
| | exposed in ambient condition for 5 hours. | | | |
| Form 1 of | 1.0 (200° C.) | 226.6 | Anhydrate | 0.62 |
| Compound 1-A | | | | |
| Form 3 of | 1.0 (150° C.) | 165.0 | Anhydrate | 7.1 |
| Compound 1-B | | | | |

The results indicated that Form 3 of Compound 1-B was substantially more soluble than the other forms tested.

### Example 9: Kinetic Solubility

Kinetic solubility studies of Form A, Form C, Form 1 of Compound 1-A, and Form 3 of Compound 1-B were performed using water, SGF, FaSSIF, and FeSSIF with sampling time at 1, 4 and 24 hrs at 37° C. Approximately 40 mg of solids were suspended into 3 mL of each medium with dosing concentration at approximately 10 mg/mL. Prepared suspension was equilibrated via rolling (25 rpm). Approximately 1.0 mL of suspension was extracted at each time point and centrifuged to obtain precipitate and supernatants. Solubility and pH were tested for the supernatants after filtration, and isolated precipitate was tested by XRPD. The kinetic solubility results are shown in Table 25.

Form 3 of Compound 1-B showed the highest solubility in water (7.1 mg/mL) when compared to Form A (0.003 mg/mL), Form C (0.002 mg/mL) and Form 1 of Compound 1-A (0.62 mg/mL).

Form A and Form C displayed no form change after equilibrium in the four buffers for 24 hours.

Form 1 of Compound 1-A displayed no form change after equilibrium in water for 1 hour. Form 1 of Compound 1-A transferred to Form A after equilibrium in SGF or FeSSIF for 1 hour. Form 1 of Compound 1-A transferred to Type A after equilibrium in FaSSIF for 1, 4, or 24 hours.

Form 3 of Compound 1-B transferred to Form A after equilibrium in SGF or FaSSIF for 1, 4 or 24 hours. Form 3 of Compound 1-B transferred to Form A after equilibrium in FeSSIF for 1 hour. Form 3 of Compound 1-B transferred to Form A after equilibrium in water for 24 hours.

TABLE 25

| | | 1 h | | | 4 h | | | 24 h | | |
|---|---|---|---|---|---|---|---|---|---|---|
| Sample | Buffer | S | pH | FC | S | pH | FC | S | pH | FC |
| Form A | H₂O | 0.003 | 8.1 | N | 0.001 | 8.3 | N | 0.003 | — | N |
| Form C | (pH = 8.4) | 0.004 | 7.4 | N | 0.002 | 7.9 | N | 0.002 | 7.0 | N |
| Form 1 of | | 0.67 | 8.6 | N | 0.78 | 9.0 | N | 0.62 | 9.5 | Y |
| Compound 1-A | | | | | | | | | | |
| Form 3 of | | 7.3 | 9.2 | G | 7.3 | 9.5 | G | 7.1 | 9.7 | Y |
| Compound 1-B | | | | | | | | | | |
| Form A | SGF | 0.022 | 1.9 | N | 0.022 | 2.0 | N | 0.022 | 2.0 | N |
| Form C | (pH = 1.8) | 0.005 | 2.0 | N | 0.005 | 2.0 | N | 0.004 | 2.0 | N |
| Form 1 of | | 0.60* | 7.8 | Y | 0.91* | 8.2 | Y | 1.00* | 8.4 | Y |
| Compound 1-A | | | | | | | | | | |
| Form 3 of | | 1.64* | 7.9 | Y | 1.22* | 8.2 | Y | 1.84* | 8.4 | Y |
| Compound 1-B | | | | | | | | | | |
| Form A | FaSSIF | 0.029 | 6.6 | N | 0.026 | 6.7 | N | 0.017 | 6.8 | N |
| Form C | (pH = 6.5) | 0.040 | 6.5 | N | 0.043 | 6.5 | N | 0.042 | 6.5 | N |
| Form 1 of | | 1.90* | 7.0 | Y | 0.37* | 7.6 | Y | 4.08* | 7.8 | Y |
| Compound 1-A | | | | | | | | | | |
| Form 3 of | | 1.68* | 7.0 | Y | 3.15* | 7.7 | Y | 5.08* | 7.8 | Y |
| Compound 1-B | | | | | | | | | | |
| Form A | FeSSIF | 0.050 | 5.3 | N | 0.050 | 5.5 | N | 0.080 | 5.7 | N |
| Form C | (pH = 5.0) | 0.050 | 5.0 | N | 0.054 | 5.1 | N | 0.061 | 5.1 | N |
| Form 1 of | | 0.35* | 6.4 | Y | 0.42* | 6.6 | Y | 0.41* | 6.5 | Y |
| Compound 1-A | | | | | | | | | | |
| Form 3 of | | 0.46* | 6.4 | Y | 0.50* | 6.4 | Y | 0.49* | 6.6 | Y |
| Compound 1-B | | | | | | | | | | |

S: solubility (mg/mL), FC: form change, N: solid form not changed, Y: solid form changed, —: Sample was not enough for test.
*Suspension was obtained after centrifugation under 6000 rpm for 5 minutes and filtration though 0.22 μm PTFE filter for two times.
SGF = Simulated Gastric Fluid;
FaSSIF: Fasted State Simulated Intestinal Fluid;
FeSSIF: Fed State Simulated Intestinal Fluid

### Example 10: Physical and Chemical Stability

The physical and chemical stability of Form 1 of Compound 1-A, Form 3 of Compound 1-B, and Form A were studied under 25° C./60% RH and 40° C./75% RH. Approximately 20 mg of a solid sample of each crystalline form was added to a HPLC vial (open dish) and then stored under 25° C./60% RH or 40° C./75% RH. After one week, the samples were removed from the test conditions and analyzed by HPLC and XRPD to assess chemical and physical stability, respectively. Form 1 of Compound 1-A, Form 3 of Compound 1-B, and Form A displayed no significant degradation and/or form change under conditions of 25° C./60% RH and 40° C./75% RH as shown in Table 26.

TABLE 26

| | | | Stability results | | |
|---|---|---|---|---|---|
| | | Initial | (Open for 1 week) | | |
| Solid form | | Purity | Purity | Form | |
| (ID) | Conditions | (area %) | (area %) | change | Appearance |
| Form 1 of | 25° C./60% RH | 99.79 | 99.73 | No | No |
| Compound 1-A | 40° C./75% RH | | 99.73 | change | change |
| Form 3 of | 25° C./60% RH | 99.77 | 99.72 | | |
| Compound 1-B | 40° C./75% RH | | 99.73 | | |
| Form A | 25° C./60% RH | 99.78 | 99.82 | | |
| | 40° C./75% RH | | 99.75 | | |

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosure.

What is claimed is:

1. A crystalline form of a compound of Formula (I): 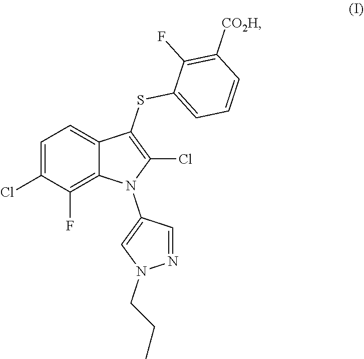 [SMILES: CCCn1cc(-n2c(Cl)c(Sc3cccc(C(=O)O)c3F)c3ccc(Cl)c(F)c32)cn1]
or a solvate thereof.

2. The crystalline form of claim 1, wherein the crystalline form exhibits an X-ray powder diffraction pattern comprising at least one characteristic peak, wherein said characteristic peak is selected from the group consisting of approximately 7.2, 10.4, 12.0, 14.5, 17.9, 21.5, 21.8, 22.3, 23.5, 25.0, 25.8, 27.2, 27.7, 29.8, 31.6, and 36.7 degrees 2θ.

3. The crystalline form of claim 2, wherein the crystalline form exhibits an X-ray powder diffraction pattern comprising at least three characteristic peaks, wherein said characteristic peaks are selected from a group consisting of approximately 7.2, 10.4, 12.0, 14.5, 17.9, 21.5, 21.8, 22.3, 23.5, 25.0, 25.8, 27.2, 27.7, 29.8, 31.6, and 36.7 degrees 2θ.

4. The crystalline form of claim 3, wherein the crystalline form exhibits an X-ray powder diffraction pattern comprising characteristic peaks at approximately 7.2, 10.4, 17.9, 21.5, 21.8, and 25.8 degrees 2θ.

5. The crystalline form of claim 1, wherein the crystalline form exhibits an X-ray powder diffraction pattern comprising at least one characteristic peaks, wherein said characteristic peaks are selected from a group consisting of approximately 6.1, 7.1, 8.8, 10.5, 12.1, 14.0, 14.4, 15.0, 17.0, 18.3, 21.1, 21.9, 23.1, 24.1, 24.4, 25.4, 27.0, 27.5, and 28.2 degrees 2θ.

6. The crystalline form of claim 5, wherein the crystalline form exhibits an X-ray powder diffraction pattern comprising at least three characteristic peaks, wherein said characteristic peaks are selected from a group consisting of approximately 6.1, 7.1, 8.8, 10.5, 12.1, 14.0, 14.4, 15.0, 17.0, 18.3, 21.1, 21.9, 23.1, 24.1, 24.4, 25.4, 27.0, 27.5, and 28.2 degrees 2θ.

7. The crystalline form of claim 6, wherein the crystalline form exhibits an X-ray powder diffraction pattern comprising characteristic peaks at approximately 6.1, 7.1, 8.8, 14.4, 17.0, 18.3, 21.1, 21.9, 23.1, 25.4, and 28.2 degrees 2θ.

8. The crystalline form of claim 1, wherein the crystalline form exhibits an X-ray powder diffraction pattern comprising at least one characteristic peak, wherein said characteristic peak is selected from the group consisting of approximately 6.7, 9.6, 11.2, 13.3, 16.1, 17.7, 18.1, 18.9, 19.3, 20.0, 20.6, 21.5, 24.5, 24.9, 27.5, 28.3, 33.7, and 33.9 degrees 2θ.

9. The crystalline form of claim 8, wherein the crystalline form exhibits an X-ray diffraction powder diffraction pattern comprising at least three characteristic peaks, wherein said characteristic peaks are selected from a group consisting of approximately 6.7, 9.6, 11.2, 13.3, 16.1, 17.7, 18.1, 18.9, 19.3, 20.0, 20.6, 21.5, 24.5, 24.9, 27.5, 28.3, 33.7, and 33.9 degrees 2θ.

10. The crystalline form of claim 9, wherein the crystalline form exhibits an X-ray powder diffraction pattern comprising characteristic peaks at approximately 6.7, 11.2, 13.3, 16.1, 17.7, 18.1, 18.9, 19.3, 20.0, 20.6, 21.5, and 24.9 degrees 2θ.

11. The crystalline form of claim 1, wherein the crystalline form exhibits an X-ray powder diffraction pattern comprising at least one characteristic peaks, wherein said characteristic peaks are selected from a group consisting of approximately 6.7, 11.2, 12.0, 13.7, 16.0, 17.5, 17.9, 18.7, 20.0, 20.4, 21.4, 22.9, 24.1, 24.7, 25.1, 25.7, 26.1, 27.3, 27.7, 28.3, 28.8, 29.9, 30.3, 30.8, 33.8, 34.3, 35.4, and 38.1 degrees 2θ.

12. The crystalline form of claim 11, wherein the crystalline form exhibits an X-ray powder diffraction pattern comprising at least three characteristic peaks, wherein said characteristic peaks are selected from a group consisting of approximately 6.7, 11.2, 12.0, 13.7, 16.0, 17.5, 17.9, 18.7, 20.0, 20.4, 21.4, 22.9, 24.1, 24.7, 25.1, 25.7, 26.1, 27.3, 27.7, 28.3, 28.8, 29.9, 30.3, 30.8, 33.8, 34.3, 35.4, and 38.1 degrees 2θ.

13. The crystalline form of claim 12, wherein the crystalline form exhibits an X-ray powder diffraction pattern comprising characteristic peaks at approximately 6.7, 11.2, 12.0, 13.7, 16.0, 17.5, 17.9, 18.7, 20.4, 21.4, 22.9, 24.1, 24.7, 29.9, 30.3, and 30.8 degrees 2θ.

14. The crystalline form of claim 1, wherein the crystalline form exhibits an X-ray powder diffraction pattern comprising at least one characteristic peaks, wherein said characteristic peaks are selected from a group consisting of approximately 6.7, 7.3, 9.9, 12.0, 13.3, 15.0, 17.3, 18.6, 19.9, 20.5, 21.3, 23.2, 23.8, 24.6, 25.8, 26.3, 26.7, 28.3, 29.2, 29.8, 31.6, 33.5, 35.0, and 38.7 degrees 2θ.

15. The crystalline form of claim 14, wherein the crystalline form exhibits an X-ray powder diffraction pattern comprising at least three characteristic peaks, wherein said characteristic peaks are selected from a group consisting of approximately 6.7, 7.3, 9.9, 12.0, 13.3, 15.0, 17.3, 18.6, 19.9, 20.5, 21.3, 23.2, 23.8, 24.6, 25.8, 26.3, 26.7, 28.3, 29.2, 29.8, 31.6, 33.5, 35.0, and 38.7 degrees 2θ.

16. The crystalline form of claim 15, wherein the crystalline form exhibits an X-ray powder diffraction pattern comprising characteristic peaks at approximately 6.7, 9.9, 12.0, 13.3, 17.3, 18.6, 19.9, 23.2, 24.6, 26.3, 29.2, 29.8, 35.0, and 38.7 degrees 2θ.

17. The crystalline form of any one of claims 1 to 16, where the crystalline form is a hydrate.

18. The crystalline form of any one of claims 1 to 16, where the crystalline form is anhydrous.

19. The crystalline form of any one of claims 1 to 16, where the crystalline form is a solvate.

20. The crystalline form of any one of claims 1 to 16, where the crystalline form is an ethanol solvate.

21. The crystalline form of any one of claims 1 to 16, where the crystalline form is a chloroform solvate.

22. A composition comprising a crystalline form of any one of claims 1 to 21, wherein greater than 50% by weight of the total amount of the compound of Formula (I) in the composition is the crystalline form.

23. The composition of any one of claims 1 to 21, wherein greater than 85% by weight of the total amount of the compound of Formula (I) in the composition is the crystalline form.

24. The composition of any one of claims 1 to 21, wherein greater than 90% by weight of the total amount of the compound of Formula (I) in the composition is the crystalline form.

25. An arginine salt form of a compound of Formula (I): 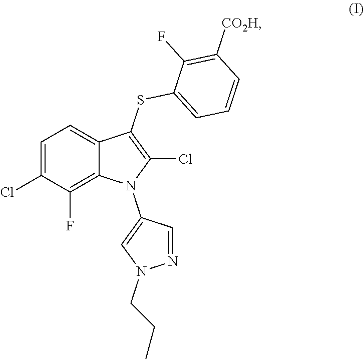 [SMILES: CCCn1cc(-n2c(Cl)c(Sc3cccc(C(=O)O)c3F)c3ccc(Cl)c(F)c32)cn1]
or a solvate thereof.

26. The arginine salt of claim 25 in crystalline form.

27. The arginine salt of claim 26, wherein the crystalline form exhibits an X-ray powder diffraction pattern comprising at least one characteristic peak, wherein said characteristic peak is selected from the group consisting of approximately 5.9, 8.8, 10.4, 12.3, 14.6, 17.1, 17.5, 19.4, 20.7, 25.7, 27.0, and 28.1 degrees 2θ.

28. The arginine salt of claim 27, wherein the crystalline form exhibits an X-ray diffraction powder diffraction pattern comprising at least three characteristic peaks, wherein said characteristic peaks are selected from a group consisting of approximately 5.9, 8.8, 10.4, 12.3, 14.6, 17.1, 17.5, 19.4, 20.7, 25.7, 27.0, and 28.1 degrees 2θ.

29. The arginine salt of claim 28, wherein the crystalline form exhibits an X-ray powder diffraction pattern comprising characteristic peaks at approximately 5.9, 8.8, 10.4, 12.3, 14.6, 17.1, 17.5, and 19.4 degrees 2θ.

30. The arginine salt of claim 26, wherein the crystalline form exhibits an X-ray powder diffraction pattern comprising at least one characteristic peak, wherein said characteristic peak is selected from the group consisting of approximately 5.7, 8.6, 10.3, 10.5, 12.1, 12.4, 14.2, 14.5, 16.6, 16.9, 17.8, 19.3, 20.0, 20.5, 25.1, and 25.6 degrees 2θ.

31. The arginine salt of claim 30, wherein the crystalline form exhibits an X-ray diffraction powder diffraction pattern comprising at least three characteristic peaks, wherein said characteristic peaks are selected from a group consisting of 5.7, 8.6, 10.3, 10.5, 12.1, 12.4, 14.2, 14.5, 16.6, 16.9, 17.8, 19.3, 20.0, 20.5, 25.1, and 25.6 degrees 2θ.

32. The arginine salt of claim 31, wherein the crystalline form exhibits an X-ray powder diffraction pattern comprising characteristic peaks at approximately 5.7, 8.6, 10.3, 10.5, 12.1, 12.4, 14.2, 14.5, 16.6, and 16.9 degrees 2θ.

33. The arginine salt of any one of claims 25-32, where the salt is anhydrous.

34. A composition comprising the arginine salt of any one of claims 25 to 33, wherein greater than 50% by weight of the total amount of the compound of Formula (I) in the composition is the arginine salt.

35. A composition comprising the arginine salt of any one of claims 25 to 33, wherein greater than 85% by weight of the total amount of the compound of Formula (I) in the composition is the arginine salt.

36. A composition comprising the arginine salt of any one of claims 25 to 33, wherein greater than 90% by weight of the total amount of the compound of Formula (I) in the composition is the arginine salt.

37. An N-methylglucamine salt form of a compound of Formula (I): 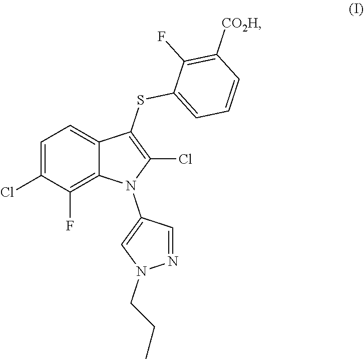 [SMILES: CCCn1cc(-n2c(Cl)c(Sc3cccc(C(=O)O)c3F)c3ccc(Cl)c(F)c32)cn1]
or a solvate thereof.

38. The N-methylglucamine salt of claim 37 in crystalline form.

39. The N-methylglucamine salt of claim 38, wherein the crystalline form exhibits an X-ray powder diffraction pattern comprising at least one characteristic peak, wherein said characteristic peak is selected from the group consisting of approximately 8.3, 9.5, 10.5, 10.8, 12.2, 12.5, 14.2, 14.5, 16.4, 16.8, 18.4, 18.6, 19.0, 20.3, and 25.4 degrees 2θ.

40. The N-methylglucamine salt of claim 39, wherein the crystalline form exhibits an X-ray diffraction powder diffraction pattern comprising at least three characteristic peaks, wherein said characteristic peaks are selected from a group consisting of approximately 8.3, 9.5, 10.5, 10.8, 12.2, 12.5, 14.2, 14.5, 16.4, 16.8, 18.4, 18.6, 19.0, 20.3, and 25.4 degrees 2θ.

41. The N-methylglucamine salt of claim 40, wherein the crystalline form exhibits an X-ray powder diffraction pattern comprising characteristic peaks at approximately 8.3, 9.5, 10.5, 10.8, 12.2, 12.5, 14.2, 14.5, 16.4, 16.8, and 18.6 degrees 2θ.

42. The N-methylglucamine salt of claim 38, wherein the crystalline form exhibits an X-ray powder diffraction pattern comprising at least one characteristic peak, wherein said characteristic peak is selected from the group consisting of approximately 8.5, 9.6, 11.6, 14.1, 16.1, 16.6, 19.1, 19.4, 19.7, 24.2, and 25.4 degrees 2θ.

43. The N-methylglucamine salt of claim 42, wherein the crystalline form exhibits an X-ray diffraction powder diffraction pattern comprising at least three characteristic peaks, wherein said characteristic peaks are selected from a group consisting of approximately 8.5, 9.6, 11.6, 14.1, 16.1, 16.6, 19.1, 19.4, 19.7, 24.2, and 25.4 degrees 2θ.

44. The N-methylglucamine salt of claim 43, wherein the crystalline form exhibits an X-ray powder diffraction pattern comprising characteristic peaks at approximately 8.5, 9.6, 11.6, 16.1, 16.6, 19.1, 19.4, 24.2, and 25.4 degrees 2θ.

45. The N-methylglucamine salt of any one of claims 37 to 44, where the salt is anhydrous.

46. A composition comprising the N-methylglucamine salt of any one of claims 37 to 45, wherein greater than 50% by weight of the total amount of the compound of Formula (I) in the composition is the N-methylglucamine salt.

47. A composition comprising the N-methylglucamine salt of any one of claims 37 to 45, wherein greater than 85% by weight of the total amount of the compound of Formula (I) in the composition is the N-methylglucamine salt.

48. A composition comprising the N-methylglucamine salt of any one of claims 37 to 45, wherein greater than 90% by weight of the total amount of the compound of Formula (I) in the composition is the N-methylglucamine salt.

49. A pharmaceutical composition, comprising the composition of any one of claim 22-24, 34-36, or 46-48 and a pharmaceutically acceptable excipient.

50. A method of treating fibrosis, comprising administering the pharmaceutical composition of claim 49 to a subject in need thereof.
